(19) United States
(12) Patent Application Publication
(10) Pub. No.: US 2026/0294899 A1
(43) Pub. Date: Oct. 1, 2026

(54) METHODS OF TREATING ERYTHROPOIETIC PROTOPORPHYRIA OR X-LINKED PROTOPORPHYRIA WITH BITOPERTIN

(71) Applicant: Disc Medicine, Inc., Watertown, MA (US)

(72) Inventors: Melanie Chin, Watertown, MA (US); William Jacob Savage, Watertown, MA (US); John Quisel, Watertown, MA (US); John Chapin, Watertown, MA (US)

(21) Appl. No.: 19/635,556

(22) Filed: Mar. 31, 2026

Related U.S. Application Data
(60) Provisional application No. 63/781,100, filed on Mar. 31, 2025.
(60) Provisional application No. 63/821,983, filed on Jun. 11, 2025.
(60) Provisional application No. 63/889,073, filed on Sep. 26, 2025.

(51) Int. Cl. A61K 31/496; A61K 9/28; A61P 1/16; A61P 3/00

(52) U.S. Cl. CPC A61K 31/496; A61K 9/2813; A61K 9/2826; A61K 9/284; A61K 9/286; A61K 9/2866; A61P 1/16; A61P 3/00

(57) ABSTRACT

The present disclosure is directed to methods of using bitopertin for preventing or treating erythropoietic protoporphyria (EPP) and/or X-linked protoporphyria (XLPP), and related syndromes thereof.

### RELATED APPLICATIONS

This application claims the benefit of priority to U.S. Provisional Patent Application No. 63/781,100, filed Mar. 31, 2025, U.S. Provisional Patent Application No. 63/821,983 filed Jun. 11, 2025, and U.S. Provisional Patent Application No. 63/889,073, filed Sep. 26, 2025, which applications are hereby incorporated by reference in their entirety.

### BACKGROUND

Erythropoietic protoporphyria (EPP) is prevalent globally and affects about 5,000-10,000 individuals worldwide (Michaels et al. 2010). EPP is considered the most common form of porphyria in children. Erythropoietic protoporphyria is a form of porphyria, which varies in severity and can be very painful. It arises from a deficiency in the enzyme ferrochelatase, leading to abnormally high levels of protoporphyrin IX in red blood cells (erythrocytes), plasma, skin, and liver. PPIX accumulation in the skin can be activated by light, thereby produce free radicals and causing cellular injury and inflammation which can result in painful phototoxic reactions. Erythropoietic protoporphyria (EPP) is due to an inherited or acquired deficiency in the activity of the enzyme ferrochelatase. X-linked protoporphyria (XLPP) is due to an inherited increase in the activity of delta-aminolevulinic acid synthase-2 (ALAS2). Enzymes that cause both EPP and XLPP are in the heme biosynthetic pathway. EPP and XLPP are nearly identical clinically.

Progressive accumulation of high concentrations of PPIX can result in crystallization, biliary stones and architectural damage within the hepatobiliary system. Liver disease can be a devastating and life-threatening complication associated with EPP and/or XLPP. Approximately 20% of patients with EPP develop gallstones, with up to 5% of patients progressing to liver failure. Naik, H., et al., JIMD Rep. 2025 Apr. 20; 66 (3): e70015. doi: 10.1002/jmd2.70015. eCollection 2025.

In a study comparing the symptoms and impacts of phototoxic reactions, health-related quality of life, and healthcare utilization between EPP patients with and without liver disease, patients with liver disease reported a higher prevalence of comorbidities, including anemia (76% vs. 55%), gallstones (44% vs. 28%), depression (56% vs. 22%), and anxiety (52% vs. 22%). Naik, H., et al., 2025. Sixty-four percent of those with liver disease rated their general health “much worse” than those without EPP, compared with 35% of those without liver disease. Naik, H., et al. 2025. In addition to general health burdens, EPP patients with liver disease report a decreased quality of life compared to those without liver disease-self-reporting a higher frequency of prodromal symptoms, greater negative impact of phototoxic reactions on their ability to perform daily activities, more than 3-fold greater mean number of work hours missed, more than 2-fold greater mean number of school hours missed, and an increased number of physician visits or hospitalizations over a 12-month period. Naik, H., et al., 2025.

Only one treatment is approved for adults with EPP in the US and the EU: afamelanotide, a subcutaneously administered α-melanocyte-stimulating hormone analogue. There are no approved therapies for patients <18 years of age, and a recent registry study that enrolled 7 participants between 15 and 17 years of age failed to demonstrate a favorable benefit-risk profile for afamelanotide in the adolescent population (EMA Withdrawal Notification). EPP exacts a particular toll on children; limiting outside socialization and physical activity and/or missing daytime school events negatively impacts psychosocial development. Therefore, pediatric patients with EPP have a high unmet medical need for treatments.

In addition, while afamelanotide can improve the ability of a patient with EPP to tolerate sunlight, the pivotal study in the US did not show a reduction in the occurrence of phototoxic reactions or decrease the pathological PPIX levels that cause EPP (Langendonk J G, et al. N Engl J Med. 2015; 373 (1): 48-59). Another limitation of afamelanotide is its mode of administration; the surgical implantation procedure must be repeated every 2 months, representing a meaningful burden to patients. Moreover, administration of afamelanotide is restricted to designated porphyria centers, where prescribing physicians can also evaluate the impact of dermatological changes due to use of afamelanotide, such as nevi and other skin abnormalities or risk of skin cancer. The travel to designated prescribers adds additional burden for patients and caregivers and can limit access to treatment. Thus, there remains a high unmet need for new therapies in EPP.

### SUMMARY OF THE DISCLOSURE

The present disclosure provides a method of treating erythropoietic protoporphyria (EPP) or X-linked protoporphyria (XLPP) comprising administering 60 mg of bitopertin to a subject in need thereof for a treatment period of at least 17 weeks thereby reducing the rate of phototoxic reactions compared to the rate observed in treatment with placebo by 75% or more.

The present disclosure provides a method of treating EPP or XLPP comprising administering 60 mg of bitopertin to a subject in need thereof for a treatment period of at least 17 weeks thereby resulting in a statistically significant improvement in pain-free light exposure in the subject. In certain such embodiments, the average daily time of pain-free light exposure is increased by at least 20%. In other such embodiments, the improvement in pain-free light exposure occurs after at least 4 weeks of treatment. In certain embodiments of the foregoing, the improvement in pain-free light exposure is sustained for 2 weeks.

The present disclosure provides a method of treating erythropoietic protoporphyria (EPP) or X-linked protoporphyria (XLPP) comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

The present disclosure provides a method of treating EPP or XLPP comprising administering 60 mg of bitopertin to a subject in need thereof for a treatment period of at least 17 weeks thereby resulting in a statistically significant improvement in pain-free light exposure in the subject.

The present disclosure provides a method of increasing sunlight tolerance in a subject with EPP or XLPP comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

The present disclosure provides a method of reducing liver damage in a subject with EPP or XLPP comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

The present disclosure provides a method of reducing protoporphyrin IX levels in a subject with EPP or XLPP comprising administering 60 mg of bitopertin to a subject in need thereof for a treatment period of at least 17 weeks thereby reducing any one or both of i) the rate of phototoxic reactions compared to the rate observed in treatment with placebo by 75% or more, or ii) reducing alanine aminotransferase (ALT) levels in the subject by at least 5%.

The present disclosure provides a method of treating erythropoietic protoporphyria (EPP) or X-linked protoporphyria (XLPP) in a subject in need thereof comprising a) administering 30 mg of bitopertin to the subject for at least 14 days; and b) administering 60 mg of bitopertin to the subject for a treatment period of at least 17 weeks; thereby reducing any one or both of i) the rate of phototoxic reactions compared to the rate observed in treatment with placebo by 75% or more, or ii) reducing alanine aminotransferase (ALT) levels in the subject by at least 5%.

The present disclosure provides a method of treating EPP or XLPP comprising administering 60 mg of bitopertin to a subject in need thereof for a treatment period of at least 17 weeks wherein the subject's heme and hemoglobin levels are maintained in comparison to reduced levels of heme and hemoglobin observed in a healthy subject administered bitopertin.

The present disclosure provides a method of treating EPP or XLPP comprising administering 60 mg of bitopertin to a subject in need thereof for at least 17 weeks wherein the subject maintains liver function.

The present disclosure provides a method of treating EPP comprising administering 60 mg of bitopertin to a subject age 12 and older in need thereof for at least 17 weeks thereby resulting in one or more of the following: a reduction in PPIX levels; a reduction in the rate of phototoxic reactions; an increase in average time of pain-free light exposure; an increase in average time to prodrome; an increase in proportion of prodrome-free weekly light exposure challenges; an increase in mean cumulative total time in light without pain; an improvement in the EPP Impact Questionnaire (EPIQ); an improvement in the Patient Global Impression of Pain (PGIC); and an improvement in the Patient Global Impression of Severity (PGIS).

The present disclosure provides a method of treating EPP comprising administering 60 mg of bitopertin to a geriatric subject in need thereof for at least 17 weeks thereby resulting in one or more of the following: a reduction in PPIX levels; a reduction in the rate of phototoxic reactions; an increase in average time of pain-free light exposure; an increase in average time to prodrome; an increase in proportion of prodrome-free weekly light exposure challenges; an increase in mean cumulative total time in light without pain an improvement in the EPP Impact Questionnaire (EPIQ); an improvement in the Patient Global Impression of Pain (PGIC); and an improvement in the Patient Global Impression of Severity (PGIS).

In certain embodiments of methods of the present disclosure, the 60 mg of bitopertin is administered as two 30 mg doses. In certain embodiments of methods of the present disclosure, the bitopertin is administered once daily.

The present disclosure provides a method of treating EPP or XLPP in a subject in need thereof comprising administering to the subject a daily therapeutically effective dosage of bitopertin for a treatment period of at least 6 weeks, wherein pain free light exposure is increased to 5 or more hours compared to observed pain free light exposure in treatment with placebo.

The present disclosure provides a method of treating EPP or XLPP in a subject comprising administering a daily therapeutically effective dosage of bitopertin for a treatment period of at least 17 weeks, wherein whole blood PPIX is decreased and no dose dependent decrease in hemoglobin is observed compared to treatment in a subject without EPP.

In certain embodiments of the methods of the present disclosure, the subject is a pediatric subject. In some embodiments, the subject is less than 18 years of age. In some embodiments, the subject is between 12 and 18 years of age.

The present disclosure provides a method of treating EPP or XLPP in a pediatric subject comprising administering a therapeutically effective dosage of bitopertin. In certain embodiments, the subject is less than 18 years of age. In some embodiments, the subject is between 12 and 18 years of age. In some embodiments, the therapeutically effective dosage of bitopertin is 30 mg of bitopertin. In some embodiments, the daily therapeutically effective dosage of 30 mg of bitopertin is administered to the subject daily for at least 14 days. In some embodiments, the therapeutically effective dosage of bitopertin is increased to 60 mg of bitopertin after at least 14 days.

In certain embodiments of the methods and uses provided herein, the amount of or therapeutically effective dosage of bitopertin is modified for concomitant use with one or more CYP3A4 inhibitors. In some embodiments, the amount of or therapeutically effective dosage of bitopertin is reduced to 30 mg of bitopertin for concomitant use with one or more CYP3A4 inhibitors. In some embodiments, the one or more CYP3A4 inhibitors is a moderate CYP3A4 inhibitor. In some embodiments, the one or more CYP3A4 inhibitors is a strong CYP3A4 inhibitor. In some embodiments, the one or more CYP3A4 inhibitors is selected from the group consisting of: ketoconazole, erythromycin, and carbamazepine. In some embodiments, the subject has hepatic impairment, and the subject is administered a modified amount of or modified therapeutically effective dosage of 30 mg of bitopertin once daily. In some embodiments, the mild hepatic impairment is classified based upon the Child-Pugh scoring system. In some embodiments, the hepatic impairment is mild hepatic impairment. In some embodiments, the subject has a Child-Pugh score of 5 to 6. In some embodiments, the subject is in Child-Pugh class A. In some embodiments, the hepatic impairment is moderate hepatic impairment. In some embodiments, the subject has a Child-Pugh score of 7 to 9. In some embodiments, the subject is in Child-Pugh class B. In some embodiments, the hepatic impairment is severe hepatic impairment. In some embodiments, the subject has a Child-Pugh score of 10 to 15. In some embodiments, the subject is in Child-Pugh class C.

In certain embodiments of methods of the present disclosure, a median maximum pain score due to phototoxic reactions over the treatment period is reduced. In certain such embodiments, the median maximum pain score due to phototoxic reactions is reduced by at least 1 unit on a pain score rating scale.

In certain embodiments of methods of the present disclosure, a number of phototoxic reactions over the treatment period is reduced. In certain such embodiments, the number of phototoxic reactions over the treatment period is reduced by at least 50%.

In certain embodiments of methods of the present disclosure, one or more complications of EPP or XLPP are treated or prevented. In other embodiments of methods of the present disclosure, progression rate and/or severity of one or more complications of EPP or XLPP are reduced. In certain such embodiments, the one or more complications of EPP, XLPP, or CEP is selected from the group consisting of: edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, terminal phase liver disease, erythrodontia, hypercellular bone marrow, myelodysplasia, thrombocytopenia, hydrops fetalis and/or death in utero.

In certain embodiments of methods of the present disclosure, the subject has EPP.

In certain embodiments of methods of the present disclosure, the subject has XLPP.

In certain embodiments of methods of the present disclosure, the bitopertin is administered orally. In certain embodiments, the bitopertin is formulated as an immediate release tablet. In certain such embodiments, the immediate release tablet comprises one or more components selected from the group consisting of lactose monohydrate, maize starch, croscarmellose sodium, povidone K30, microcrystalline cellulose, talc, magnesium stearate, water, and a film coating comprising one or more of partially hydrolyzed polyvinyl alcohol, macrogol/polyethylene glycol, cellulose, talc, titanium dioxide, and iron oxide yellow. In certain embodiments of the foregoing, the immediate release tablet comprises 30 mg of bitopertin. In certain embodiments, the bitopertin is formulated for storage at 20° C. to 25° C. In certain embodiments, the bitopertin is formulated for storage at 68° F. to 77° F. In some embodiments, excursions from storage are permitted between 15° C. and 30° C. In some embodiments, excursions from storage are permitted between 59° F. and 86° F.

In certain embodiments of methods of the present disclosure, the subject does not experience changes in skin pigmentation.

In certain embodiments of methods of the present disclosure, the subject experiences less than 5% of adverse events associated with anemia. In certain such embodiments, the adverse events associated with anemia are selected from shortness of breath, difficulty breathing, weakness, fatigue, dizziness, lightheadedness, headaches, nausea, irregular heartbeat, chest pain, tinnitus, swelling, and/or jaundice.

In certain embodiments of methods of the present disclosure, the subject experiences less than 5% of adverse events associated with the central nervous system (CNS). In certain such embodiments, the adverse events associated with the CNS are selected from drowsiness, dizziness, impaired concentration, vertigo, headaches, nausea, insomnia, ataxia, and coma.

In certain embodiments of methods of the present disclosure, the administration ameliorates hepatic toxicity.

In certain embodiments of methods of the present disclosure, the administration reduces the incidence of hepatobiliary disease. In certain embodiments of methods of the present disclosure, the administration reduces the progression of hepatobiliary disease.

In certain embodiments of methods of the present disclosure, the subject's PPIX levels decrease by at least 50% (e.g., 50%, 55%, 60%, 65%, 70%, 75%, 80%, 85%, 90%, 95%, or at least 100%) and the subject's heme levels decrease no more than 10%. In certain embodiments of methods of the present disclosure, the subject's heme levels decrease no more than 10%. In certain embodiments of methods of the present disclosure, the subject's PPIX levels decrease while the subject's heme levels are substantially maintained.

In certain embodiments of methods of the present disclosure, the subject's PPIX levels decrease by at least 50% (e.g., 50%, 55%, 60%, 65%, 70%, 75%, 80%, 85%, 90%, 95%, or at least 100%) and the subject's hemoglobin levels decrease no more than 10%. In certain embodiments of methods of the present disclosure, the subject's hemoglobin levels decrease no more than 10%. In certain embodiments of methods of the present disclosure, the subject's PPIX levels decrease while the subject's hemoglobin levels are substantially maintained.

In certain embodiments of methods of the present disclosure, the subject has increased free-protoporphyrin IX levels in erythrocytes. In certain embodiments, the method decreases protoporphyrin IX levels in the erythrocytes of the subject.

In certain embodiments of methods of the present disclosure, the method decreases free-protoporphyrin IX levels in the subject. In certain such embodiments, the method decreases free-protoporphyrin IX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments of methods of the present disclosure, the subject has increased protoporphyrin IX levels in stool. In certain embodiments, the method decreases protoporphyrin IX levels in stool of the subject. In certain such embodiments, the method decreases protoporphyrin IX levels in the stool of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments of methods of the present disclosure, protoporphyrin IX (PPIX) synthesis is inhibited in vivo.

In certain embodiments of methods of the present disclosure, the subject's plasma porphyrin fluoresces at a peak of 634 nm when illuminated with blue light (e.g., 400-420 nm light). In certain embodiments, the subject's plasma porphyrin fluoresces at a peak between 626 nm and 634 nm when illuminated with blue light (e.g., 400-420 nm light).

In certain embodiments of methods of the present disclosure, the subject's skin porphyrin fluoresces at a peak of 632 nm when illuminated with blue light (e.g., 400-420 nm light). In certain embodiments, the subject's skin porphyrin fluoresces at a peak between 626 nm and 634 nm when illuminated with blue light (e.g., 400-420 nm light).

In certain embodiments of methods of the present disclosure, the subject has increased protoporphyrin IX levels in the skin. In certain embodiments, the method decreases protoporphyrin IX levels in the skin of the subject. In certain such embodiments, the method decreases protoporphyrin IX levels in the skin of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In certain embodiments of methods of the present disclosure, the subject has greater than 0.2 FluoDerm Units (FDU) of protoporphyrin IX levels in the skin. In certain embodiments, the subject has greater than 1.0 FDU of protoporphyrin IX levels in the skin. In certain embodiments, the subject has between 1.0 FDU and 2.5 FDU of protoporphyrin IX levels in the skin. In other embodiments, the subject has greater than 2.5 FDU of protoporphyrin IX levels in the skin. In certain embodiments of methods of the present disclosure, the method decreases protoporphyrin IX levels in the skin of the subject to less than 0.5 FDU, to less than 1.0 FDU, to less than 1.5 FDU, to less than 2.0 FDU, or to less than 2.5 FDU.

In certain embodiments of methods of the present disclosure, zinc protoporphyrin IX (ZPPIX) synthesis is inhibited in vivo.

In certain embodiments of methods of the present disclosure, 5-aminolevulinic acid (5-ALA) synthesis is inhibited in vivo.

In certain embodiments of methods of the present disclosure, the subject in need thereof is 12 years and older. In certain embodiments of methods of the present disclosure, the subject is between 12 and 18 years old.

In certain embodiments of methods of the present disclosure, the subject in need thereof is a geriatric subject.

In certain embodiments of methods of the present disclosure, the total pain-free time in light of the subject increases as compared to the total pain-free time in light prior to treatment.

The present disclosure provides a method of treating liver disease associated with EPP or XLPP in a subject comprising administering 60 mg of bitopertin to a subject to a subject in need thereof for a treatment period of at least four weeks.

The present disclosure provides a method of treating protoporphyrin IX (PPIX) induced liver damage comprising administering 60 mg of bitopertin to a subject in need thereof for a treatment period of at least four weeks.

The present disclosure provides a method of treating hepatopathy comprising administering 60 mg of bitopertin to a subject in need thereof for a treatment period of at least four weeks.

The present disclosure provides a method of decreasing liver protoporphyrin IX concentrations comprising administering 60 mg of bitopertin to a subject in need thereof for a treatment period of at least four weeks.

The present disclosure provides a method of reducing hepatobiliary biomarkers of liver damage comprising administering 60 mg of bitopertin to a subject in need thereof for a treatment period of at least four weeks.

The present disclosure provides a method of increasing sunlight tolerance in a subject with EPP or XLPP comprising administering to the subject 60 mg of bitopertin for a treatment period of at least 17 weeks resulting in a statistically significant increase in sunlight tolerance without statistically significant abuse potential.

In certain embodiments of methods of the present disclosure, the subject has EPP.

In certain embodiments of methods of the present disclosure, the subject has XLPP.

In certain embodiments of methods of the present disclosure, the bitopertin is administered orally. In certain embodiments, the bitopertin is formulated as an immediate release tablet. In certain such embodiments, the immediate release tablet comprises one or more components selected from the group consisting of lactose monohydrate, maize starch, croscarmellose sodium, povidone K30, microcrystalline cellulose, talc, magnesium stearate, water, and a film coating comprising one or more of partially hydrolyzed polyvinyl alcohol, macrogol/polyethylene glycol, cellulose, talc, titanium dioxide, and iron oxide yellow. In certain embodiments of the foregoing, the immediate release tablet comprises 30 mg of bitopertin.

In certain embodiments of methods of the present disclosure, administration of the bitopertin alleviates one or more symptoms of liver disease. In certain such embodiments, the one or more symptoms of liver disease is selected from the group consisting of ascites, encephalopathy, hepatocellular carcinoma, jaundice, and peripheral edema.

In certain embodiments of methods of the present disclosure, the subject has or is at risk of developing hepatic fibrosis.

In certain embodiments of methods of the present disclosure, the subject is at risk of progression to cirrhosis. In certain such embodiments, the subject has a low-risk, mid-risk, or high-risk of progression to cirrhosis. In certain embodiments of the foregoing, administration of the bitopertin decreases the risk of progression to cirrhosis. In certain embodiments of methods of the present disclosure the subject is at risk of progression to cirrhosis, the risk of progression to cirrhosis is determined with an Enhanced Liver Fibrosis (ELF) test. In certain such embodiments, the subject has an ELF score of less than 9.80. In other such embodiments, the subject has an ELF score between 9.80 and 11.29. In other such embodiments, the subject has an ELF score greater than 11.29. In certain embodiments of the foregoing, the method decreases the subject's ELF score to less than 9.80. In other embodiments of the foregoing, the method decreases the subject's ELF score to less than 11.29. In certain embodiments of methods of the present disclosure wherein the subject is at risk of progression to cirrhosis, the method prevents or delays advancement from low-risk to mid-risk of progression to cirrhosis. In other embodiments of methods of the present disclosure wherein the subject is at risk of progression to cirrhosis, method prevents or delays advancement from mid-risk to high-risk of progression to cirrhosis.

In certain embodiments of methods of the present disclosure wherein the subject has or is at risk of developing hepatic fibrosis, the severity of hepatic fibrosis is determined via ultrasound elastography. In certain embodiments of methods of the present disclosure wherein the subject has or is at risk of developing hepatic fibrosis, the severity of hepatic fibrosis is determined via magnetic resonance elastography. In certain embodiments of methods of the present disclosure wherein the subject has or is at risk of developing hepatic fibrosis, the severity of hepatic fibrosis is determined via FibroScan. In certain embodiments of the foregoing, the subject has a hepatic fibrosis score of F0, F1, F2, F3, or F4. In certain embodiments, the subject has a hepatic fibrosis score of F0 and has no scarring of the liver. In certain embodiments, the subject has a hepatic fibrosis score of F1 and has mild scarring of the liver. In certain embodiments, the subject has a hepatic fibrosis score of F2 and has moderate scarring of the liver. In certain embodiments, the subject has a hepatic fibrosis score of F3 and has severe scarring of the liver. In certain embodiments, the subject has a hepatic fibrosis score of F4 and has cirrhosis of the liver. In certain embodiments of the foregoing, the method decreases the subject's hepatic fibrosis score from F4 to F3. In certain embodiments of the foregoing, method decreases the subject's hepatic fibrosis score from F3 to F2. In certain embodiments of the foregoing, the method decreases the subject's hepatic fibrosis score from F2 to F1. In certain embodiments of the foregoing, method decreases the subject's hepatic fibrosis score from F1 to F0. In certain embodiments of the foregoing, the method prevents or delays progression of the subject's hepatic fibrosis score from F1 to F2, F3, or F4. In certain embodiments of the foregoing, the method prevents or delays progression of the subject's hepatic fibrosis score from F2 to F3 or F4. In certain embodiments of the foregoing, the method prevents or delays progression of the subject's hepatic fibrosis score from F3 to F4.

In certain embodiments of methods of the present disclosure, the subject has a liver stiffness greater than 2 kPa. In certain such embodiments, the subject has a liver stiffness greater than 7 kPa, greater than 9 kPa, greater than 11 kPa, greater than 14 kPa, or greater than 17 kPa. In certain embodiments of the foregoing, the method decreases the subject's liver stiffness to less than 17 kPa. In certain such embodiments, the method decreases the subject's liver stiffness to less than 14 kPa, to less than 11 kPa, to less than 9 kPa, or to less than 7 kPa. In certain embodiments of the foregoing, the method delays or prevents increases in the subject's liver stiffness.

In certain embodiments of methods of the present disclosure, the subject, when compared to baseline, has elevated levels one or more hepatobiliary biomarkers selected from the group consisting of aspartate aminotransferase (AST), alanine aminotransferase (ALT), alkaline phosphatase (AP), Gamma-glutamyl transpeptidase (GGT), bilirubin, alpha-2 macroglobulin (A2M), apolipoprotein A1 (ApoA1), haptoglobin (Hp), albumin, 5′-nucleotidase (5′-NT), and primary serum bile acids. In certain embodiments of the foregoing, the method reduces serum concentrations of primary bile acids. In certain such embodiments, the method reduces serum primary bile acid levels by at least 5% in the subject, by at least 10% in the subject, or by at least 15% in the subject. In certain embodiments of the foregoing, the method reduces ALT levels in the subject by at least 5%, by at least 10%, or by at least 15%. In certain embodiments of the foregoing, the method reduces AST levels in the subject by at least 5%, by at least 10%, or by at least 15%. In certain embodiments of the foregoing, the method reduces AP concentrations in the subject by at least 5%, by at least 10%, or by at least 15%. In certain embodiments of the foregoing, the method reduces GGT concentrations in the subject by at least 5%, by at least 10%, or by at least 15%. In certain embodiments of the foregoing, the method reduces serum total bilirubin (TBR) concentrations in the subject by at least 5%, by at least 10%, or by at least 15%. In certain embodiments of the foregoing, the method reduces serum A2M concentrations in the subject by at least 5%, by at least 10%, or by at least 15%. In certain embodiments of the foregoing, the method decreases serum ApoA1 concentrations in the subject by at least 5%, by at least 10%, or by at least 15%. In certain embodiments of the foregoing, the method increases serum Hp concentrations in the subject by at least 5%, by at least 10%, or by at least 15%. In certain embodiments of the foregoing, the method increases serum albumin levels in the subject by at least 5%, by at least 10%, or by at least 15%. In certain embodiments of the foregoing, the method reduces serum 5′-NT concentrations in the subject by at least 5%, by at least 10%, or by at least 15%.

In certain embodiments of methods of the present disclosure, the subject has a FIB-4 index less than 1.00 or less than 1.30. In certain embodiments of methods of the present disclosure, the subject has a FIB-4 index greater than 2.65 or greater than 3.25. In certain embodiments of the foregoing, the method decreases the subject's FIB-4 index to less than 1.3, to less than 2.65, or to less than 3.25.

In certain embodiments of methods of the present disclosure, the subject has an APRI score greater than 0.7 or greater than 1.0. In certain embodiments of methods of the present disclosure, the subject has an APRI score greater than 1.5 or greater than 2.0. In certain embodiments of the foregoing, the method decreases the subject's APRI score to less than 2.0, to less than 1.5, or to less than 1.0.

In certain embodiments of methods of the present disclosure, the subject has increased concentrations of porphyrins in the liver relative to baseline. In certain embodiments of methods of the present disclosure, the subject has increased concentration of porphyrins in bile relative to baseline. In certain embodiments of the foregoing, the porphyrins comprise PPIX. In certain embodiments of the foregoing methods, the method reduces the concentration of porphyrins by at least 5%, at least 10%, at least 15%, at least 20%, at least 30%, at least 40%, or at least 50%.

In certain embodiments of methods of the present disclosure, the treatment period is at least 29 days, at least 71 days, at least 121 days, at least 177 days, at least 233 days, or at least 289 days.

In certain embodiments of methods of the present disclosure, bitopertin penetrates the subject's CNS without statistically significant abuse potential. In certain embodiments, bitopertin has CNS activity. In certain embodiments, bitopertin has CNS activity without statistically significant abuse potential. In certain embodiments, bitopertin is CNS active without statistically significant abuse potential.

In certain embodiments, the administration of bitopertin does not induce a statistically significant abuse potential in the subject.

In certain embodiments, the administration of bitopertin does not induce any one or more of a drug liking response, drug liking at the moment response, or a take drug again response in the subject. In certain embodiments, the administration of bitopertin does not induce overall drug liking in the subject as compared to the administration diazepam as a positive control. In certain embodiments, administration of bitopertin induces an overall drug liking response comparable to placebo. In certain embodiments, administration of bitopertin does not induce overall drug liking in the subject as measured by a Visual Analogue Scale (VAS) for Overall Drug Liking. In certain embodiments, administration of bitopertin induces an overall drug liking response that is at least 40%, at least 50%, at least 60%, at least 70%, at least 80%, at least 90%, or at least 100% less than a positive control as measured by a VAS for Overall Drug Liking. In certain embodiments of the foregoing, the positive control comprises diazepam.

In certain embodiments, the subject experiences less than 10% of adverse events associated with the central nervous system (CNS). In certain embodiments, the subject experiences less than 5% of adverse events associated with the central nervous system (CNS). In certain embodiments of the foregoing, the adverse events associated with the CNS are selected from the group consisting of drowsiness, dizziness, impaired concentration, vertigo, headaches, nausea, insomnia, ataxia, and coma.

### BRIEF DESCRIPTION OF THE DRAWINGS

FIG. 1 shows PPIX reduction with a Glyl inhibitor in Fech^{m1Pas} EPP mice. See Example 2.

FIG. 2 shows the primary efficacy endpoint, percent change in whole-blood metal free PPIX, at the end of 17 weeks for each of placebo (+8%), bitopertin 20 mg (−21.6%) and bitopertin 60 mg (−40.7%). MMRM=mixed-model repeated measures; PBO=placebo; PPIX=protoporphyrin IX; WB=whole blood. Least-squares means (±SE) and p-values for percent changes in WB metal-free PPIX analysed using an MMRM to compare 20 mg and 60 mg bitopertin dose groups versus placebo. MMRM models included fixed effects for randomization stratification factor (time to prodrome <30 min or ≥30 min), baseline PPIX, treatment, visit, treatment-by-visit interactions, and a random effect for each participant. Multiple imputation was used to impute missing data. See Example 3.

FIG. 3 shows percent change in whole blood metal free PPIX at the end of 17 weeks for 60-mg dose Bitopertin within different subgroups. BMI=body mass index; EOS=end of study; MMRM=mixed-model repeated measures; LS=least squares; NC=not calculated; PPIX=protoporphyrin IX; WB=whole blood. Least-squares means and p-values for percent changes in WB metal-free PPIX at Day 121 analysis visit were analyzed using an MMRM to compare 20-mg and 60-mg bitopertin dose groups versus placebo. MMRM models included fixed effects for randomization stratification factor (time to prodrome <30 min or ≥30 min), baseline PPIX, treatment, visit, treatment-by-visit interactions, and a random effect for each participant. Multiple imputation was used to impute missing data. See Example 3.

FIG. 4 shows mean cumulative 4-month total time in sunlight without pain in hours for placebo (n=24), bitopertin at 20 mg (n=26) and bitopertin at 60 mg (n=25). An analysis of variance (ANOVA) was used to compare cumulative time in light for 20 mg and 60 mg bitopertin dose groups versus placebo. Multiple imputation was used to impute missing data. ANOVA models included fixed effects for randomization stratification factor and treatment. See Example 3.

FIG. 5 shows total pain free sunlight exposure averaged over 2-week intervals for placebo (n=24), bitopertin at 20 mg (n=26) and bitopertin at 60 mg (n=25). MMRM=mixed-model repeated measures; SE=standard error. Least-squares means for daily sunlight exposure by 2-week interval analysis visit were analyzed using MMRM to compare 20 mg and 60 mg bitopertin dose groups versus placebo. MMRM models included fixed effects for treatment, randomization stratification factor (time to prodrome <30 min or ≥30 min), visit, treatment-by-visit interactions, and a random effect for each participant. Multiple imputation was used to impute missing data that are plotted here. See Example 3.

FIG. 6 shows a correlation between change in pain-free sunlight exposure and changes in PPIX as a scatter plot of change in total pain-free sunlight exposure versus percent change in PPIX at the end of study relative to baseline (n=75) randomized to 20-mg bitopertin, 60-mg bitopertin, or placebo. Change in total pain-free sunlight exposure was calculated by taking the difference between the last 2-week interval of the study and the 2-week screening period prior to randomization (baseline). See Example 3.

FIG. 7 shows placebo-corrected pain-free sunlight exposure and PPIX change for 60-mg bitopertin over 18 weeks. Data for participants randomized to the bitopertin 60-mg group. Least-squares means results for percent change in WB metal-free PPIX levels and placebo-corrected total pain-free time in sunlight averaged over 2-week intervals are obtained from MMRM models. MMRM models included fixed effects for randomization stratification factor (time to prodrome <30 min or ≥30 min), baseline PPIX (for PPIX MMRM model only), treatment, visit, treatment-by-visit interactions, and a random effect for each participant. Multiple imputation was used to impute missing data. See Example 3.

FIG. 8 shows pain-free sunlight exposure and PPIX change for 60-mg bitopertin over 18 weeks. Least-squares means results for percent change in WB metal-free PPIX levels and total pain-free time in sunlight averaged over 2-week intervals are obtained from MMRM models. MMRM models included fixed effects for randomization stratification factor (time to prodrome <30 min or ≥30 min), baseline PPIX (for PPIX MMRM model only), treatment, visit, treatment-by-visit interactions, and a random effect for each participant. Multiple imputation was used to impute missing data. See Example 3.

FIG. 9 shows average time to prodrome averaged over 2-week intervals. LS=least squares; MMRM=mixed-model repeated measures. Shown are LS means for time to prodrome data from weekly sunlight-exposure challenges were averaged over a 2-week period, including cumulative time in sunlight challenges where the participant did not report a prodrome, and were analyzed using MMRM for 20-mg bitopertin, 60-mg bitopertin, and placebo groups. MMRM models included fixed effects for treatment, randomization stratification factor, visit, and treatment-by-visit interactions. See Example 3.

FIG. 10 shows incidence rate ratio of phototoxic reactions vs placebo. Incidence rate ratio was estimated from a negative-regression model with offset of cumulative daylight exposure over the entire treatment period, and covariates for treatment, baseline number of new phototoxic reactions, and time to prodrome stratification factor. See Example 3.

FIG. 11 shows time course over the study of phototoxic reactions for placebo. Bitopertin (20 mg and 60 mg). 20 mg=20 mg bitopertin; 60 mg=60 mg bitopertin, D60=Study Day 60; PBO=placebo; TRT=treatment. Each row depicts a participant reporting new phototoxic reactions during the double-blind period and corresponding study day for new reaction (in red). Participants without a phototoxic reaction throughout the entire study are not shown. The last column summarizes the number of phototoxic reactions reported for each participant during the last 60 days of the study. See Example 3.

FIG. 12 shows comparisons between bitopertin treatment groups (20 mg and 60 mg) and placebo for both PGIC and PGIS were performed using an ordinal regression model that included covariates for randomization stratification factor and baseline severity. EPP=erythropoietic protoporphyria; PGIC=Patient Global Impression of Change; PGIS=Patient Global Impression of Severity. See Example 3.

FIG. 13 shows an overview of the observed decrease in hemoglobin (Hgb) over 120 days after daily administration of placebo, 10, 30, or 60 mg Bitopertin. This decrease was variable and overall comparable between placebo and the 10 mg group as well as between 30 mg and 60 mg groups. On Day 120, the estimated difference to placebo (g/L) with 90% CIs amounted to −4.5 (−10.7 to 1.8), −13.4 (−19.7 to −7.2), and −18.5 (−24.9 to −12.2) after 10, 30, and 60 mg bitopertin, respectively. In the 30 mg and 60 mg dose groups, Hgb was estimated to be 9.3% (90% CI −13.5, −5.0) and 13.0% (90% CI −17.0, −8.7) lower than in the placebo group. See Example 4.

FIG. 14 shows mean hemoglobin levels remained unchanged relative to baseline with bitopertin (both 20 mg and 60 mg doses) in participants with EPP. See Example 4.

FIG. 15 shows mild, dose-dependent increases in serum iron were observed with bitopertin treatment in EPP subjects. See Example 4.

FIG. 16 shows the change in whole blood PPIX over 169 days (e.g., 24 weeks or 6 months) for bitopertin (20 mg and 60 mg) (FIG. 16A) and in adolescent and adult subpopulations (FIG. 16B) as compared to baseline. Least-squares means (±SE) and p-values for percent changes in WB metal-free PPIX analyzed using an MMRM model to compare 20 mg, 60 mg, and combined bitopertin dose groups (left) and for adult and adolescent subpopulations (right). MMRM models included fixed effects for randomization stratification factor (time to prodrome <30 min or ≥30 min), baseline PPIX, treatment, visit, treatment-by-visit interactions, and a random effect for each participant. Missing PPIX data were not imputed. MMRM, mixed model repeated measures; PPIX, protoporphyrin IX; WB, whole blood. See Example 5.

FIG. 17 shows total pain-free time in sunlight averaged over two-week intervals for bitopertin (20 mg and 60 mg) dose groups (FIG. 17A) and in adolescent and adult subpopulations (FIG. 17B) as compared to baseline. Total daily sunlight exposure from 10:00 AM to 6:00 PM on days without pain averaged over 2-week periods and analyzed using an MMRM model to compare 20 mg and 60 mg bitopertin dose groups (left) and adult and adolescent subpopulations (right). MMRM models included fixed effects for randomization stratification factor (time to prodrome <30 min or ≥30 min), baseline PPIX, treatment, baseline total pain-free sunlight exposure (during 14-day screening period), visit, treatment-by-visit interactions, and a random effect for each participant. MMRM, mixed model repeated measures; PPIX, protoporphyrin IX. See Example 5.

FIG. 18 shows aggregate measures of sunlight tolerance at the time of screening and on treatment. ^{a}As assessed with a daily diary; ^{b}As assessed with a weekly sunlight challenge; Summed across all participants. Percentages calculated relative to total number of days with sunlight exposure (left) or total number of weekly sunlight exposure challenges (right) from all study participants (n=22) during screening or while receiving bitopertin (20 mg and 60 mg dose groups combined). See Example 5.

FIG. 19 shows the Phase 3 study schema. See Example 5.

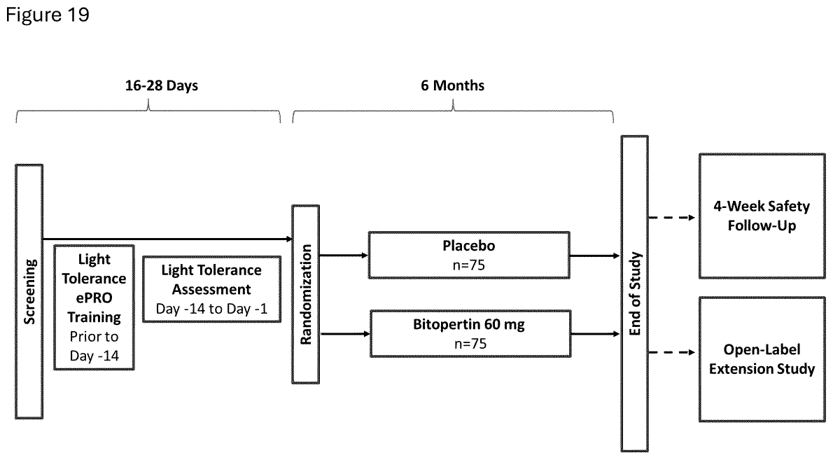

FIG. 20 shows data from a subset of clinical trial participants who either received 60 mg of bitopertin continuously (AURORA or BEACON and HELIOS) or were randomized to placebo in AURORA and subsequently received 60 mg bitopertin in HELIOS. Study day is calculated relative to clinical trial day 1. The change from baseline is calculated relative to trial baseline. The abbreviations ALT and SE mean alanine aminotransferase and standard error, respectively. See, Example 7.

### DETAILED DESCRIPTION OF THE DISCLOSURE

The present disclosure provides methods of treating Erythropoietic protoporphyria (EPP) in a subject in need thereof. The present disclosure also provides methods of treating X-linked protoporphyria (XLPP) in a subject in need thereof. In certain aspects, the present disclosure provides methods of treating EPP and XLPP in a subject in need thereof. In certain aspects, the present disclosure provides methods of treating EPP or XLPP in a subject in need thereof. In certain aspects, the present disclosure provides methods of treating EPP and/or XLPP in a subject in need thereof.

The methods as disclosed herein are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human. In certain embodiments of the methods and uses disclosed herein, the subject is a subject in need thereof. In some embodiments, the subject is an adult, adolescent (e.g., pediatric patient ages 12 years and older), geriatric, child, infant, or pregnant woman. In some embodiments, the subject is an adolescent subject. In certain embodiments, the subject is a pediatric subject 12 years and older. In some embodiments, the adolescent subject is less than 18 years old. In some embodiments, the adolescent subject is between 12 and 18 years old. In some embodiments, the subject is a geriatric subject. In some embodiments, the geriatric subject is age 65 or older. In a preferred embodiment of the methods provided herein, the subject in need thereof is age 12 and older. In certain embodiments, the subject is an adult or a pediatric subject 12 years and older.

Unless defined otherwise, all technical and scientific terms have the same meaning as is commonly understood by one of ordinary skill in the art to which the embodiments disclosed belongs.

As used herein, the terms “a” or “an” means that “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the phrase “pharmaceutically acceptable” means those compounds, materials, compositions, and/or dosage forms which are, within the scope of sound medical judgment, suitable for use in contact with tissues of humans and animals. In some embodiments, “pharmaceutically acceptable” means approved by a regulatory agency of the Federal or a state government or listed in the U.S. Pharmacopeia or other generally recognized pharmacopeia for use in animals, and more particularly in humans.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be determined based on the age, health, size and sex of the subject. Optimal amounts can also be determined based on monitoring of the subject's response to treatment.

As used herein, the terms “treat,” “treated,” or “treating” mean both therapeutic treatment and prophylactic measures wherein the object is to slow down (lessen) an undesired physiological condition, disorder or disease, or obtain beneficial or desired clinical results. Beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response without excessive levels of side effects. Treatment also includes prolonging survival as compared to expected survival if not receiving treatment. Thus, “treatment of erythropoietic protoporphyria” or “treating erythropoietic protoporphyria” means an activity that alleviates or ameliorates any of the primary phenomena or secondary symptoms associated with the erythropoietic protoporphyria or other condition described herein.

In certain embodiments, the methods provided herein comprise administering a glycine transporter 1 (GlyT1) inhibitor to a subject. In certain embodiments, the methods provided herein comprise administering bitopertin to a subject. Bitopertin is an oral small molecule inhibitor of glycine transporter 1, a modulator of heme biosynthesis.

Bitopertin is a protoporphyrin IX (PPIX)-lowering GlyT1 inhibitor indicated for the treatment of erythropoietic protoporphyria and X-linked protoporphyria in adults. Bitopertin is also indicated for the treatment of erythropoietic protoporphyria and X-linked protoporphyria pediatric patients aged 12 years and older. Bitopertin is also indicated for the treatment of erythropoietic protoporphyria and X-linked protoporphyria geriatric patients aged 65 years and older. Bitopertin can be used for decreasing PPIX levels in adults with erythropoietic protoporphyria or X-linked protoporphyria. Bitopertin can also be used for decreasing PPIX levels in pediatric patients aged 12 years and older with erythropoietic protoporphyria or X-linked protoporphyria. Bitopertin can also be used for decreasing PPIX levels in geriatric patients aged 65 years and older with erythropoietic protoporphyria or X-linked protoporphyria.

In some embodiments of the uses and methods as disclosed herein, the bitopertin is administered in a therapeutically effective amount. The bitopertin described herein can be administered either alone or in combination (concurrently or serially) with other pharmaceuticals. For example, the bitopertin can be administered in combination with other drugs for the treatment of EPP or XLPP. Examples of other pharmaceuticals or medicaments are known to one of skill in the art and include, but are not limited to those described herein.

Bitopertin can be administered in combination with one or more CYP3A4 inhibitors. The CYP3A4 enzyme is a member of the cytochrome P450 family of oxidizing enzymes found in the liver. Cytochrome P450 enzymes, such as CYP3A4, oxidize small organic molecules in the body, including certain drugs. In certain embodiments of the methods and uses provided herein, bitopertin can be administered in combination with one or more CYP3A4 inhibitors. In certain embodiments of the methods and uses provided herein, bitopertin can be administered concomitantly with one or more CYP3A4 inhibitors. In certain embodiments of the methods and uses provided herein, the amount of or therapeutically effective dosage of bitopertin is modified for concomitant use with one or more CYP3A4 inhibitors. In certain embodiments, the amount of or therapeutically effective dosage of bitopertin is reduced to 30 mg of bitopertin for concomitant use with one or more CYP3A4 inhibitors. In certain embodiments, the one or more CYP3A4 inhibitors is a moderate CYP3A4 inhibitor. In certain embodiments, the one or more CYP3A4 inhibitors is a strong CYP3A4 inhibitor. In certain embodiments, the one or more CYP3A4 inhibitors is selected from the group consisting of: ketoconazole, erythromycin, and carbamazepine. In some embodiments, the one or more CYP3A4 inhibitors is ketoconazole. In some embodiments, the one or more CYP3A4 inhibitors is erythromycin. In some embodiments, the one or more CYP3A4 inhibitors is carbamazepine.

The amount of bitopertin to be administered is that amount which is therapeutically effective. In certain embodiments of the methods disclosed herein, 60 mg of bitopertin is administered to the subject in need thereof. In certain such embodiments, the 60 mg of bitopertin is administered as two doses, such as two approximately simultaneous doses, of 30 mg of bitopertin each. In certain embodiments of the methods disclosed herein, at least 60 mg of bitopertin is administered to the subject in need thereof. In certain embodiments of the foregoing, the at least 60 mg of bitopertin (e.g., 60 mg of bitopertin, such as two 30 mg doses of bitopertin administered approximately simultaneously) is administered once daily. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician). The selection of the specific dose regimen can be selected or adjusted or titrated by the clinician according to methods known to the clinician to obtain the desired clinical response.

An amount of bitopertin as provided herein can be administered for any treatment period. “Treatment period”, as used herein, is given its usual and ordinary meaning, namely, a period of time during which a drug, e.g., bitopertin, is to be administered to a subject. A treatment period can be measured in days, weeks, and/or years.

In some embodiments, the methods provided herein comprise administering bitopertin to a subject in need thereof for a treatment period of at least 7 days. In some embodiments, the methods provided herein comprise administering bitopertin to a subject in need thereof for a treatment period of at least 14 days. In some embodiments, the methods provided herein comprise administering bitopertin to a subject in need thereof for a treatment period of at least 15 days. In some embodiments, the methods provided herein comprise administering bitopertin to a subject in need thereof for a treatment period of at least 21 days. In some embodiments, the methods provided herein comprise administering bitopertin to a subject in need thereof for a treatment period of at least 28 days. In some embodiments, the methods provided herein comprise administering bitopertin to a subject in need thereof for a treatment period of at least 30 days. In some embodiments, the methods provided herein comprise administering bitopertin to a subject in need thereof for a treatment period of at least 35 days. In some embodiments, the methods provided herein comprise administering bitopertin to a subject in need thereof for a treatment period of at least 42 days. In some embodiments, the methods provided herein comprise administering bitopertin to a subject in need thereof for a treatment period of at least 60 days. In some embodiments, the methods provided herein comprise administering bitopertin to a subject in need thereof for a treatment period of at least 119 days. In some embodiments, the methods provided herein comprise administering bitopertin to a subject in need thereof for a treatment period of at least 120 days. In some embodiments, the methods provided herein comprise administering bitopertin to a subject in need thereof for a treatment period of at least 121 days. In some embodiments, the methods provided herein comprise administering bitopertin to a subject in need thereof for a treatment period of at least 150 days. In some embodiments, the methods provided herein comprise administering bitopertin to a subject in need thereof for a treatment period of at least 160 days. In some embodiments, the methods provided herein comprise administering bitopertin to a subject in need thereof for a treatment period of at least 169 days.

In some embodiments, the methods provided herein comprise administering bitopertin to a subject in need thereof for a treatment period of at least 4 weeks. In some embodiments, the methods provided herein comprise administering bitopertin to a subject in need thereof for a treatment period of at least 5 weeks. In some embodiments, the methods provided herein comprise administering bitopertin to a subject in need thereof for a treatment period of at least 6 weeks. In some embodiments, the methods provided herein comprise administering bitopertin to a subject in need thereof for a treatment period of at least 7 weeks. In some embodiments, the methods provided herein comprise administering bitopertin to a subject in need thereof for a treatment period of at least 8 weeks. In some embodiments, the methods provided herein comprise administering bitopertin to a subject in need thereof for a treatment period of at least 9 weeks. In some embodiments, the methods provided herein comprise administering bitopertin to a subject in need thereof for a treatment period of at least 10 weeks. In some embodiments, the methods provided herein comprise administering bitopertin to a subject in need thereof for a treatment period of at least 11 weeks. In some embodiments, the methods provided herein comprise administering bitopertin to a subject in need thereof for a treatment period of at least 12 weeks. In some embodiments, the methods provided herein comprise administering bitopertin to a subject in need thereof for a treatment period of at least 13 weeks. In some embodiments, the methods provided herein comprise administering bitopertin to a subject in need thereof for a treatment period of at least 14 weeks. In some embodiments, the methods provided herein comprise administering bitopertin to a subject in need thereof for a treatment period of at least 15 weeks. In some embodiments, the methods provided herein comprise administering bitopertin to a subject in need thereof for a treatment period of at least 16 weeks. In some embodiments, the methods provided herein comprise administering bitopertin to a subject in need thereof for a treatment period of at least 17 weeks. In some embodiments, the methods provided herein comprise administering bitopertin to a subject in need thereof for a treatment period of at least 18 weeks. In some embodiments, the methods provided herein comprise administering bitopertin to a subject in need thereof for a treatment period of at least 19 weeks. In some embodiments, the methods provided herein comprise administering bitopertin to a subject in need thereof for a treatment period of at least 20 weeks. In some embodiments, the methods provided herein comprise administering bitopertin to a subject in need thereof for a treatment period of at least 21 weeks. In some embodiments, the methods provided herein comprise administering bitopertin to a subject in need thereof for a treatment period of at least 22 weeks. In some embodiments, the methods provided herein comprise administering bitopertin to a subject in need thereof for a treatment period of at least 23 weeks. In some embodiments, the methods provided herein comprise administering bitopertin to a subject in need thereof for a treatment period of at least 24 weeks. In some embodiments, the methods provided herein comprise administering bitopertin to a subject in need thereof for a treatment period of at least 25 weeks. In some embodiments, the methods provided herein comprise administering bitopertin to a subject in need thereof for a treatment period of at least 30 weeks. In some embodiments, the methods provided herein comprise administering bitopertin to a subject in need thereof for a treatment period of at least 40 weeks. In some embodiments, the methods provided herein comprise administering bitopertin to a subject in need thereof for a treatment period of at least 50 weeks. In some embodiments, the methods provided herein comprise administering bitopertin to a subject in need thereof for a treatment period of at least 52 weeks.

In some embodiments, the methods provided herein comprise administering bitopertin to a subject in need thereof for a treatment period of at least 1 year. In some embodiments, the methods provided herein comprise administering bitopertin to a subject in need thereof for a treatment period of at least 2 years. In some embodiments, the methods provided herein comprise administering bitopertin to a subject in need thereof for a treatment period of at least 3 years. In some embodiments, the methods provided herein comprise administering bitopertin to a subject in need thereof for a treatment period of at least 4 years. In some embodiments, the methods provided herein comprise administering bitopertin to a subject in need thereof for a treatment period of at least 5 years.

In some embodiments, the present disclosure provides methods of treating EPP or XLPP in a pediatric subject comprising administering a therapeutically effective dosage of bitopertin. In certain of the methods and uses disclosed herein, the subject is an adolescent (e.g., pediatric patient ages 12 years and older), child, or infant. In some embodiments, the subject is an adolescent subject. In certain embodiments, the subject is a pediatric subject 12 years and older. In some embodiments, the adolescent subject is less than 18 years old. In some embodiments, the adolescent subject is between 12 and 18 years old. In some embodiments, the therapeutically effective dosage of bitopertin is 60 mg of bitopertin. In some embodiments, the therapeutically effective dosage of bitopertin is 30 mg of bitopertin. In some embodiments, the therapeutically effective dosage of 30 mg of bitopertin is administered to the subject daily for at least 14 days. In some embodiments, the therapeutically effective dosage of bitopertin is increased to 60 mg of bitopertin after at least 14 days. In some embodiments, the amount of or therapeutically effective dosage of bitopertin is modified for concomitant use with one or more CYP3A4 inhibitors.

The amount of a compound described herein that will be effective in the treatment and/or prevention of a particular disease, condition, or disorder will depend on the nature and extent of the disease, condition, or disorder, and can be determined by standard clinical techniques. Bitopertin as utilized according to the methods disclosed herein can be formulated as disclosed in WO 2015/082367, the contents of which are incorporated herein by reference. In a preferred embodiment of the methods provided herein, the bitopertin is formulated as an immediate release tablet. In further embodiments, the immediate release tablet comprises one or more components selected from the group consisting of lactose monohydrate, maize starch, croscarmellose sodium, povidone K30, microcrystalline cellulose, talc, magnesium stearate, water, and a film coating comprising one or more of partially hydrolyzed polyvinyl alcohol, macrogol/polyethylene glycol, cellulose, talc, titanium dioxide, and iron oxide yellow. In further embodiments, immediate release tablet comprises 30 mg of bitopertin. For example, in certain embodiments, bitopertin tablets comprise the excipients as provided in Table 1 below. In some embodiments, immediate release tablet is formulated for storage at 20° C. to 25° C. In some embodiments, immediate release tablet is formulated for storage at 68° F. to 77° F. In some embodiments, excursions from storage are permitted between 15° C. and 30° C. In some embodiments, excursions from storage are permitted between 59° F. and 86° F.

TABLE 1
30 mg Bitopertin tablet

| | mg/tablet |
|---|---|
| Bitopertin | 30 |
| Lactose Monohydrate | 53.75 |
| Maize Starch | 37.5 |
| Croscarmellose Sodium | 3.75 |
| Povidone 30 | 6.25 |
| Microcrystalline Cellulose | 15.0 |
| Talc | 3.0 |
| Magnesium Stearate | 0.75 |
| Total Weight Core | 150.0 |
| Opadry Film-Coating | 5.0 |
| Total Film-Coated Weight | 155.0 |

As used herein, “inhibition” can refer to inhibition of a specific activity. The activity of bitopertin can be measured by any method known in the art including but not limited to the methods described herein.

Erythropoietic protoporphyria (EPP) and X-linked protoporphyria (XLPP) are erythropoietic cutaneous porphyrias characterized by acute non-blistering photosensitivity, intolerance to sunlight, and significantly reduced quality of life. EPP is caused by a partial deficiency in ferrochelatase (FECH), which catalyzes the final step in the heme biosynthesis pathway. FECH deficiency increases levels of metal-free erythrocyte PPIX (also referred to herein as “free-protoporphyrin IX” and “PPIX”). XLPP is typically caused by C-terminal deletions in the ALAS2 gene which result in a gain-of-function mutation. These gain-of-function mutations increase the enzymatic activity of ALAS2 and cause an accumulation of both metal-free and zinc-bound PPIX. Both EPP and XLPP result in an accumulation of PPIX in erythrocytes and other tissues or biological fluids (e.g., skin, liver, bile, or stool). PPIX, which is lipophilic and eliminated via bile, is hepatotoxic at high concentrations.

Patients with EPP or XLPP usually develop photosensitivity during early childhood. Patients frequently present with symptoms of burning, itching, pain erythema, and edema on sun-exposed areas. Cutaneous symptoms are sometimes associated with abnormal liver enzyme activities, hepatobiliary injury, such as jaundice and liver cirrhosis, iron deficiency, and corresponding microcytic anemia.

EPP symptom onset typically begins in early childhood and is present throughout the lives of patients and includes severe and exaggerated sensitivity of the skin in response to typically harmless light (i.e., photosensitivity), including sunlight and artificial fluorescent light, wherein patients can present with acute and severe phototoxicity within minutes of sun exposure. Phototoxicity manifests as symptoms including burning pain, purpura, erythema, and edema. Longer exposure to visible (mostly blue violet) and long-wave ultraviolet light leads to phototoxic reactions characterized by intense, debilitating pain, which no type of analgesic (including narcotics) can relieve. All these effects can last for several days after phototoxic exposure. Starting in childhood, patients with EPP actively modify their lifestyle to avoid unnecessary sun exposure. As a result, children with EPP are at a higher risk of social isolation and rejection due to their physical inability to participate in various activities, such as field trips, outdoor events, and other social interactions that could put them in danger of sunlight exposure. Consequently, EPP can have meaningful psychosocial consequences and negative impacts on quality of life.

Other effects of excess systemic PPIX in patients with EPP include anemia, osteopenia, and hepatobiliary disease. PPIX is lipophilic and excreted by the liver into bile. In patients with EPP, PPIX progressively accumulates in the liver, and high concentrations of PPIX can result in crystallization, forming biliary stones and causing architectural damage within the hepatobiliary system. These changes may range from mild inflammation to cholestasis to life-threatening fibrosis or cirrhosis of the liver. Approximately 20% of patients with EPP develop gallstones due to high levels of insoluble PPIX, with up to 5% of patients progressing to liver failure.

The diagnosis of EPP and XLPP can be determined by measuring the levels of total erythrocyte, free-protoporphyrin IX, and zinc-protoporphyrin IX in hemolyzed anticoagulated whole blood. A diagnosis of EPP and/or XLPP can be made based on increased levels of free-protoporphyrin IX in blood. Patients with XLPP have a significantly higher proportion of zinc-protoporphyrin IX to free-protoporphyrin IX (e.g., >25%) as compared to those with EPP (e.g., ≤15%).

The diagnosis of EPP can also be determined by measuring the level of ferrocheletase activity in a subject. Ferrocheletase is a mitochondrial enzyme that catalyzes the insertion of ferrous iron into PPIX to form heme. Ferrocheletase also catalyzes the insertion of zinc, to form zinc protoporphyrin IX (ZPPIX) from any PPIX that remains after completion of heme synthesis. In EPP, free PPIX accumulates in bone marrow reticulocytes, since formation of both heme and ZPPIX is impaired. In some embodiments, the disclosure relates to methods of a treating a subject whose ferrochelatase activity level is reduced to between 10 to 35% of the ferrocheletase activity level observed in normal subjects. In some embodiments, the disclosure relates to methods of a treating a subject whose ferrochelatase activity level is reduced to less than 50% of the ferrocheletase activity level observed in normal subjects.

XLPP has a similar phenotype to EPP, and can be differentiated based on genetic analysis of ALAS2 or by determining the enzymatic activity level of ALAS2. In some embodiments, the disclosure relates to methods of a treating a subject having a gain-of-function mutation in ALAS2. In some embodiments, the subject's ALAS2 enzyme activity is increased. Since ferrocheletase is not deficient in XLPP, some of the excess PPIX measured in erythrocytes is ZPPIX and a lower percentage (e.g., 50-85%) is metal-free. In some embodiments, the subject has increased zinc-protoporphyrin IX levels in erythrocytes. In some embodiments, the method decreases zinc-protoporphyrin IX levels in the subject's erythrocytes. In some embodiments, method decreases zinc-protoporphyrin IX levels in the subject's erythrocytes by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

In some embodiments, the present disclosure provides methods of treating Erythropoietic protoporphyria (EPP) comprising administering 60 mg of bitopertin to a subject in need thereof. The present disclosure also provides methods of treating X-linked protoporphyria (XLPP) comprising administering 60 mg of bitopertin to a subject in need thereof. In certain aspects, the present disclosure provides methods of treating EPP and XLPP comprising administering 60 mg of bitopertin to a subject in need thereof. In certain aspects, the present disclosure provides methods of treating EPP or XLPP comprising administering 60 mg of bitopertin to a subject in need thereof. In certain aspects, the present disclosure provides methods of treating EPP and/or XLPP comprising administering 60 mg of bitopertin to a subject in need thereof.

In some embodiments, the present methods relates to subjects having PPIX levels that are at least 10%, 20%, 30%, 40%, or 50% more than PPIX levels in a healthy subject prior to administration of the bitopertin. In some embodiments, the method relates to subjects having PPIX levels that are at least 10% more than PPIX levels in a healthy subject prior to administration of the bitopertin. In some embodiments, the method relates to subjects having PPIX levels that are at least 20% more than PPIX levels in a healthy subject prior to administration of the bitopertin. In some embodiments, the method relates to subjects having PPIX levels that are at least 30% more than PPIX levels in a healthy subject prior to administration of the bitopertin. In some embodiments, the method relates to subjects having PPIX levels that are at least 40% more than PPIX levels in a healthy subject prior to administration of the bitopertin. In some embodiments, the method relates to subjects having PPIX levels that are at least 50% more than PPIX levels in a healthy subject prior to administration of the bitopertin. In some embodiments, the subject has increased protoporphyrin IX levels in the stool. In some embodiments, the subject has increased protoporphyrin IX levels in the skin. In some embodiments, the subject has increased free-protoporphyrin IX levels in erythrocytes. In some embodiments, the subject has greater than 31 μmol L-1 protoporphyrin IX levels in the erythrocytes. In some embodiments, the subject has between 31 μmol L-1 and 53 μmol L-1 protoporphyrin IX levels in the erythrocytes. In some embodiments, the subject has greater than 53 μmol L-1 protoporphyrin IX levels in the erythrocytes.

The present disclosure further provides methods of inhibiting PPIX synthesis in vivo, comprising administering to a subject bitopertin. In certain aspects, the disclosure relates to methods of inhibiting PPIX synthesis in vivo, comprising administering to a subject bitopertin. In some embodiments, the disclosure relates to methods of inhibiting PPIX synthesis in vivo by at least 10% (e.g., 10%, 20%, 30%, 40%, 50%, 60%, 70%, 80%, 90%, or at least 100%). In some embodiments, the disclosure relates to methods of inhibiting PPIX synthesis in vivo by at least 20%. In some embodiments, the disclosure relates to methods of inhibiting PPIX synthesis in vivo by at least 30%. In some embodiments, the disclosure relates to methods of inhibiting PPIX synthesis in vivo by at least 40%. In some embodiments, the disclosure relates to methods of inhibiting PPIX synthesis in vivo by at least 50%. In some embodiments, the disclosure relates to methods of inhibiting PPIX synthesis in vivo by at least 60%. In some embodiments, the disclosure relates to methods of inhibiting PPIX synthesis in vivo by at least 70%. In some embodiments, the disclosure relates to methods of inhibiting PPIX synthesis in vivo by at least 80%. In some embodiments, the disclosure relates to methods of inhibiting PPIX synthesis in vivo by at least 90%. In some embodiments, the disclosure relates to methods of inhibiting PPIX synthesis in vivo by at least 100%. The present disclosure further provides methods of decreasing the rate of PPIX synthesis in vivo, comprising administering to a subject bitopertin. In certain embodiments of the methods and uses as disclosed herein inhibit PPIX accumulation directly or indirectly. In certain such embodiments, PPIX accumulation is inhibited in a dose dependent manner. For example, the present disclosure provides a method of inhibiting PPIX synthesis in vivo, decreasing the rate of PPIX synthesis in vitro, and/or inhibiting PPIX accumulation in vivo, comprising administering to a subject bitopertin. In some embodiments, the subject's heme and hemoglobin levels are maintained in comparison to reduced levels of heme and hemoglobin observed in a healthy subject administered bitopertin.

In some embodiments, the method relates to methods of decreasing free-protoporphyrin IX levels in the subject. In some embodiments, the method relates to methods of decreasing free-protoporphyrin IX levels in the subject's erythrocytes. In some embodiments, the method decreases protoporphyrin IX levels in the erythrocytes of the subject to levels less than 53 μmol L-1. In some embodiments, the method decreases protoporphyrin IX levels in the erythrocytes of the subject to levels less than 31 μmol L-1. In some embodiments, the method decreases protoporphyrin IX levels in the erythrocytes of the subject to levels less than 15 μmol L-1. In some embodiments, the method relates to decreasing protoporphyrin IX levels in the stool of the subject. In some embodiments, the method decreases protoporphyrin IX levels in the skin of the subject. In some embodiments, the method relates to methods of decreasing free-protoporphyrin IX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%). In some embodiments, the method relates to methods of decreasing free-protoporphyrin IX levels in the subject by at least 15%. In some embodiments, the method relates to methods of decreasing free-protoporphyrin IX levels in the subject by at least 20%. In some embodiments, the method relates to methods of decreasing free-protoporphyrin IX levels in the subject by at least 25%. In some embodiments, the method relates to methods of decreasing free-protoporphyrin IX levels in the subject by at least 30%. In some embodiments, the method relates to methods of decreasing free-protoporphyrin IX levels in the subject by at least 35%. In some embodiments, the method relates to methods of decreasing free-protoporphyrin IX levels in the subject by at least 40%. In some embodiments, the method relates to methods of decreasing free-protoporphyrin IX levels in the subject by at least 45%. In some embodiments, the method relates to methods of decreasing free-protoporphyrin IX levels in the subject by at least 50%. In some embodiments, the method relates to methods of decreasing free-protoporphyrin IX levels in the subject by at least 55%. In some embodiments, the method relates to methods of decreasing free-protoporphyrin IX levels in the subject by at least 60%. In some embodiments, the method relates to methods of decreasing free-protoporphyrin IX levels in the subject by at least 65%. In some embodiments, the method relates to methods of decreasing free-protoporphyrin IX levels in the subject by at least 70%. In some embodiments, the method relates to methods of decreasing free-protoporphyrin IX levels in the subject by at least 75%. In some embodiments, the method relates to methods of decreasing free-protoporphyrin IX levels in the subject by at least 80%. In some embodiments, the method relates to methods of decreasing free-protoporphyrin IX levels in the subject by at least 85%. In some embodiments, the method relates to methods of decreasing free-protoporphyrin IX levels in the subject by at least 90%. In some embodiments, the method relates to methods of decreasing free-protoporphyrin IX levels in the subject by at least 95%. In some embodiments, the method relates to methods of decreasing free-protoporphyrin IX levels in the subject by at least 100%. In some embodiments, the subject's heme and hemoglobin levels are maintained in comparison to reduced levels of heme and hemoglobin observed in a healthy subject administered bitopertin

In certain aspects, the disclosure relates to methods of treating X-linked protoporphyria (XLPP) in a subject, the method comprising administering to the subject a pharmaceutical composition comprising bitopertin, wherein the subject has increased zinc-protoporphyrin IX (ZPPIX) levels. In some embodiments, the method relates to subjects having ZPPIX levels that are at least 10%, 20%, 30%, 40%, or 50% more than ZPPIX levels in a healthy subject prior to administration of the bitopertin. In some embodiments, the method relates to subjects having ZPPIX levels that are at least 10% more than ZPPIX levels in a healthy subject prior to administration of the bitopertin. In some embodiments, the method relates to subjects having ZPPIX levels that are at least 20% more than ZPPIX levels in a healthy subject prior to administration of the bitopertin. In some embodiments, the method relates to subjects having ZPPIX levels that are at least 30% more than ZPPIX levels in a healthy subject prior to administration of the bitopertin. In some embodiments, the method relates to subjects having ZPPIX levels that are at least 40% more than ZPPIX levels in a healthy subject prior to administration of the bitopertin. In some embodiments, the method relates to subjects having ZPPIX levels that are at least 50% more than ZPPIX levels in a healthy subject prior to administration of the bitopertin. In some embodiments, the subject has increased ZPPIX levels in erythrocytes.

In certain aspects, the disclosure relates to methods of treating X-linked protoporphyria (XLPP) in a subject, the method comprising administering to the subject a pharmaceutical composition comprising bitopertin, wherein the subject has increased proportion of zinc-protoporphyrin IX (ZPPIX) to free-protoporphyrin IX (ZPPIX/PPIX ratio) as compared to those with EPP. In some embodiments, the method relates to subjects having a ZPPIX/PPIX ratio that is at least 15% (e.g., 15%, 20%, 25%, 30%, 35%, 40%, or 45%). In some embodiments, the method relates to subjects having a ZPPIX/PPIX ratio that is at least 20%. In some embodiments, the method relates to subjects having a ZPPIX/PPIX ratio that is at least 25%. In some embodiments, the method relates to subjects having a ZPPIX/PPIX ratio that is at least 30%. In some embodiments, the method relates to subjects having a ZPPIX/PPIX ratio that is at least 35%. In some embodiments, the method relates to subjects having a ZPPIX/PPIX ratio that is at least 40%. In some embodiments, the method relates to subjects having a ZPPIX/PPIX ratio that is at least 45%.

In certain aspects, the disclosure relates to methods of inhibiting zinc protoporphyrin IX (ZPPIX) synthesis in vivo, comprising administering to a subject a GlyT1 inhibitor, or a pharmaceutically acceptable salt thereof, or a prodrug of the GlyT1 inhibitor or its pharmaceutically acceptable salt. In some embodiments, the disclosure relates to methods of inhibiting ZPPIX synthesis in vivo by at least 10% (e.g., 10%, 20%, 30%, 40%, 50%, 60%, 70%, 80%, 90%, or at least 100%). In some embodiments, the disclosure relates to methods of inhibiting ZPPIX synthesis in vivo by at least 20%. In some embodiments, the disclosure relates to methods of inhibiting ZPPIX synthesis in vivo by at least 30%. In some embodiments, the disclosure relates to methods of inhibiting ZPPIX synthesis in vivo by at least 40%. In some embodiments, the disclosure relates to methods of inhibiting ZPPIX synthesis in vivo by at least 50%. In some embodiments, the disclosure relates to methods of inhibiting ZPPIX synthesis in vivo by at least 60%. In some embodiments, the disclosure relates to methods of inhibiting ZPPIX synthesis in vivo by at least 70%. In some embodiments, the disclosure relates to methods of inhibiting ZPPIX synthesis in vivo by at least 80%. In some embodiments, the disclosure relates to methods of inhibiting ZPPIX synthesis in vivo by at least 90%. In some embodiments, the disclosure relates to methods of inhibiting ZPPIX synthesis in vivo by at least 100%.

In certain aspects, the disclosure relates to methods of treating erythropoietic protoporphyria (EPP) or X-linked protoporphyria (XLPP) in a subject, the method comprising administering to the subject a pharmaceutical composition comprising bitopertin, wherein the subject has increased 5-aminolevulinic acid (5-ALA) levels. In some embodiments, the method relates to subjects having 5-ALA levels that are at least 10%, 20%, 30%, 40%, or 50% more than 5-ALA levels in a healthy subject prior to administration of the bitopertin. In some embodiments, the method relates to subjects having 5-ALA levels that are at least 10% more than 5-ALA levels in a healthy subject prior to administration of the bitopertin. In some embodiments, the method relates to subjects having 5-ALA levels that are at least 20% more than 5-ALA levels in a healthy subject prior to administration of the bitopertin. In some embodiments, the method relates to subjects having 5-ALA levels that are at least 30% more than 5-ALA levels in a healthy subject prior to administration of the bitopertin. In some embodiments, the method relates to subjects having 5-ALA levels that are at least 40% more than 5-ALA levels in a healthy subject prior to administration of the bitopertin. In some embodiments, the method relates to subjects having 5-ALA levels that are at least 50% more than 5-ALA levels in a healthy subject prior to administration of the bitopertin.

In certain aspects, the disclosure relates to methods of inhibiting 5-aminolevulinic acid (5-ALA) synthesis in vivo, comprising administering to a subject bitopertin. In some embodiments, the disclosure relates to methods of inhibiting 5-ALA synthesis in vivo by at least 10% (e.g., 10%, 20%, 30%, 40%, 50%, 60%, 70%, 80%, 90%, or at least 100%). In some embodiments, the disclosure relates to methods of inhibiting 5-ALA synthesis in vivo by at least 20%. In some embodiments, the disclosure relates to methods of inhibiting 5-ALA synthesis in vivo by at least 30%. In some embodiments, the disclosure relates to methods of inhibiting 5-ALA synthesis in vivo by at least 40%. In some embodiments, the disclosure relates to methods of inhibiting 5-ALA synthesis in vivo by at least 50%. In some embodiments, the disclosure relates to methods of inhibiting 5-ALA synthesis in vivo by at least 60%. In some embodiments, the disclosure relates to methods of inhibiting 5-ALA synthesis in vivo by at least 70%. In some embodiments, the disclosure relates to methods of inhibiting 5-ALA synthesis in vivo by at least 80%. In some embodiments, the disclosure relates to methods of inhibiting 5-ALA synthesis in vivo by at least 90%. In some embodiments, the disclosure relates to methods of inhibiting 5-ALA synthesis in vivo by at least 100%.

The present disclosure further provides use of bitopertin, in the manufacture of a formulation for the treatment of EPP or XLPP or related syndrome thereof (e.g., EPP-related syndrome or XLPP-related syndrome) in a subject. In some embodiments, the present disclosure provides use of bitopertin in the manufacture of a formulation for the treatment of EPP or XLPP in a subject. In certain embodiments of the foregoing, the formulation is administered in a therapeutically effective amount.

The present disclosure provides the use of bitopertin, in the manufacture of a pharmaceutical composition for the treatment of EPP or XLPP, or related syndrome thereof (e.g., EPP-related syndrome or XLPP-related syndrome) in a subject. In some embodiments, the present disclosure provides the use of bitopertin in the manufacture of a pharmaceutical composition for the treatment of EPP or XLPP, in a subject. In certain embodiments of the foregoing, the pharmaceutical composition further comprises a pharmaceutically acceptable carrier.

Some compounds within the GlyT1 inhibitor class have been characterized for their ability to penetrate the blood brain barrier and their effects on the central nervous system (CNS). While effective penetration into the CNS, relative peripheral drug exposure, may be a desirable profile for certain therapeutic applications (e.g., treatment of neurological disorders such as schizophrenia), one can readily envision that there could be other therapeutic applications for GlyT1 inhibitors wherein CNS penetration is not necessarily required or even undesirable due to possible off-target effects on the nervous system. Moreover, such drugs might pose a risk of abuse.

The methods described herein provide for the treatment of EPP or XLPP, or related syndrome thereof (e.g., EPP-related syndrome or XLPP-related syndrome) in a subject without statistically significant abuse potential. In some embodiments, the disclosure provides methods of increasing sunlight tolerance in a subject with EPP or XLPP. In some embodiments, the present disclosure provides a method of treating EPP or XLPP without statistically significant abuse potential. In some embodiments, the present disclosure provides a method of increasing sunlight tolerance in a subject with EPP or XLPP, without statistically significant abuse potential. In some embodiments, the present disclosure provides a method of reducing PPIX levels without statistically significant abuse potential. In some embodiments, the present disclosure provides a method of increasing sunlight tolerance in a subject with EPP or XLPP comprising administering to the subject 60 mg of bitopertin for a treatment period of at least 17 weeks resulting in a statistically significant increase in sunlight tolerance without statistically significant abuse potential.

As used herein, drug abuse refers to the intentional, non-therapeutic use of a drug product or substance, even once, to achieve a desired psychological or physiological effect. As used herein, abuse potential refers to the likelihood that abuse will occur with a particular drug product or substance with central nervous system (CNS) activity. Drug products with abuse potential may contain drug substances that have CNS activity and/or produce reinforcing effects on the CNS, such as euphoria or sedation. However, not all drugs with CNS activity carry abuse potential, and some drugs with abuse potential may act primarily through non-CNS mechanisms.

According to the FDA Guidelines, data relevant to assess whether a drug has CNS activity include one or more of chemistry studies (drug structure and ability to cross the blood brain barrier); receptor binding studies with the drug and major metabolites at CNS sites; second messenger system studies to identify functionality at binding sites; pharmacokinetic studies showing the drug's relative distribution to and penetration of the brain; and pharmacodynamic studies showing ability of the drug to induce general behavioral changes in animals and humans indicative of CNS activity. See, e.g., FDA Guidance for Industry: Assessment of Abuse Potential of Drugs (January 2017).

Drugs with CNS activity may be subject to control under the U.S. Controlled Substances Act (CSA; see, 21 U.S.C. § 811), which contains five schedules of control. Schedule I includes drugs or other substances with a high abuse potential, no currently accepted medical use, and a lack of accepted safety for use under medical supervision. Schedules II through V contain drugs or other substances with abuse potential that do have a currently accepted medical use. Placement into Schedules II through V is determined by the relative abuse potential of the drug or substance and the relative degree to which it induces psychological or physical dependence. See, e.g., FDA Guidance for Industry: Assessment of Abuse Potential of Drugs (January 2017). Unscheduled drugs or other substances do not require prescriptions or restricted pharmacy access.

In some embodiments, according to the methods provided herein, bitopertin penetrates the CNS. In some embodiments, bitopertin is CNS-penetrant. In some embodiments, bitopertin penetrates the CNS without statistically significant abuse potential. In some embodiments, bitopertin is CNS penetrant without statistically significant abuse potential. In some embodiments, bitopertin has CNS activity. In some embodiments, bitopertin is CNS-active. In some embodiments, bitopertin has CNS activity without statistically significant abuse potential. In some embodiments, bitopertin is CNS active without statistically significant abuse potential.

CNS-penetrant or CNS-active drugs or other substances may bind to one or more CNS receptors. Receptor binding studies with a drug and major metabolites at CNS sites can be used to evaluate CNS-activity both in vitro and in vivo. CNS receptors include, but are not limited to the dopamine receptor, norepinephrine receptor, serotonin receptor, GABA receptor, acetylcholine receptor, opioid receptor, NMDA receptor, and cannabinoid receptor. In some embodiments, bitopertin does not demonstrate statistically significant binding to one or more CNS receptor. In some embodiments, administration of bitopertin does not result in statistically significant binding to one or more CNS receptor. In some embodiments, bitopertin does not exhibit statistically significant binding to any CNS receptor. In some embodiments, bitopertin does not demonstrate statistically significant binding to one or more CNS receptor at concentrations up to 10 μM. In some embodiments, bitopertin does not exhibit statistically significant binding to any CNS receptor at concentrations up to 10 μM.

In some embodiments, the one or more CNS receptor is selected from the group consisting of dopamine receptor, norepinephrine receptor, serotonin receptor, GABA receptor, acetylcholine receptor, opioid receptor, NMDA receptor, and cannabinoid receptor. In some embodiments, the one or more CNS receptor is a dopamine receptor. In some embodiments, the one or more CNS receptor is a norepinephrine receptor. In some embodiments, the one or more CNS receptor is a serotonin receptor. In some embodiments, the one or more CNS receptor is a GABA receptor. In some embodiments, the one or more CNS receptor is an acetylcholine receptor. In some embodiments, the one or more CNS receptor is an opioid receptor. In some embodiments, the one or more CNS receptor is an NMDA receptor. In some embodiments, the one or more CNS receptor is a cannabinoid receptor. In a preferred embodiment, bitopertin does not exhibit statistically significant binding to the CNS receptor.

In some embodiments, bitopertin exhibits at least 300-fold greater selectivity for GlyT1 than for the one or more CNS receptor. In some embodiments, bitopertin exhibits at least 300-fold greater selectivity for GlyT1 than for a dopamine receptor. In some embodiments, bitopertin exhibits at least 300-fold greater selectivity for GlyT1 than for a norepinephrine receptor. In some embodiments, bitopertin exhibits at least 300-fold greater selectivity for GlyT1 than for a serotonin receptor. In some embodiments, bitopertin exhibits at least 300-fold greater selectivity for GlyT1 than for a GABA receptor. In some embodiments, bitopertin exhibits at least 300-fold greater selectivity for GlyT1 than for an acetylcholine receptor. In some embodiments, bitopertin exhibits at least 300-fold greater selectivity for GlyT1 than for an opioid receptor. In some embodiments, bitopertin exhibits at least 300-fold greater selectivity for GlyT1 than for an NMDA receptor. In some embodiments, bitopertin exhibits at least 300-fold greater selectivity for GlyT1 than for a cannabinoid receptor.

In some embodiments, bitopertin does not affect the uptake or release of dopamine, serotonin, or norepinephrine. In some embodiments, administration of bitopertin does not affect the uptake of dopamine. In some embodiments, administration of bitopertin does not affect the release of dopamine. In some embodiments, administration of bitopertin does not affect the uptake of serotonin. In some embodiments, administration of bitopertin does not affect the release of serotonin. In some embodiments, administration of bitopertin does not affect the uptake of norepinephrine. In some embodiments, administration of bitopertin does not affect the release of norepinephrine. In some embodiments, administration of bitopertin does not affect the uptake or release of dopamine, serotonin, or norepinephrine.

In some embodiments, bitopertin does not bind to or affect the activity of any receptor, enzyme, or channel that may lead to abuse potential. In some embodiments, administration of bitopertin does not result in binding to any receptor that may lead to abuse potential. In some embodiments, administration of bitopertin does not affect the activity of any enzyme that may lead to abuse potential. In some embodiments, administration of bitopertin does not affect the activity of any channel that may lead to abuse potential.

Abuse potential is also studied through human abuse potential studies (also referred to as HAP studies). In human abuse potential studies, standardized questionnaires are used for evaluating the subjective effects of drugs. Study participants are asked to provide information about their responses to a test drug through the use of one or more specific visual analogue scale(s) (VAS) or Addiction Research Center Inventory (ARCI) at specific time points following drug administration. 100-mm visual analog scales (VAS) can be presented as unipolar (e.g., drug liking measured on entire scale) or bipolar (e.g., drug liking and drug disliking measured with a neutral midpoint). The VAS for “Drug Liking” typically serves as the primary measure. Different or additional measures may be selected, including, but not limited to, VAS for “Overall Drug Liking,” VAS for “Drug Liking at the Moment”, and VAS for “Take Drug Again”. Alertness/drowsiness VAS and ARCI for the subscale PCAG (Pentobarbital Chlorpromazine Alcohol Group scale) can be used to evaluate sedative effects. ARCI for the subscales BG (Benzedrine Group scale) and A (Amphetamine scale) can be used to evaluate stimulant effects, including, e.g., stimulation and/or agitation. The Drug High VAS and the ARCI for the subscale MBG (Morphine Benzedrine Group scale) can be used to evaluate positive effects, including, e.g., euphoria. The ARCI for the subscale LSD (Lysergic Acid Diethylamide Group scale) can be used to evaluate negative effects, including, e.g., dysphoria and agitation. As one of skill in the art would understand, “drug liking” refers to the subjective positive effect, e.g., euphoria experienced by an individual following administration of a drug. As one of skill in the art would also understand, “drug wanting” refers to the craving that compels an individual to seek or obtain a drug.

In some embodiments, the methods provided herein do not produce a statistically significant abuse potential in the subject. In some embodiments, the method does not induce a statistically significant abuse potential in the subject. In some embodiments, the subject does not experience a statistically significant abuse potential. In some embodiments, administration of bitopertin does not produce a statistically significant abuse potential in the subject. In some embodiments, administration of bitopertin does not induce a statistically significant abuse potential in the subject. In some embodiments, the subject does not experience a statistically significant abuse potential following administration of bitopertin.

In some embodiments, administration of bitopertin induces a statistically significant abuse potential comparable to placebo. In some embodiments, administration of bitopertin does not induce a statistically significant abuse potential. In some embodiments, administration of bitopertin induces a statistically significant abuse potential that is at least 40%, at least 50%, at least 60%, at least 70%, at least 80%, at least 90%, or at least 100% less than a positive control. In some embodiments, the positive control comprises diazepam. In some embodiments, administration of bitopertin induces a statistically significant abuse potential that is at least 40%, at least 50%, at least 60%, at least 70%, at least 80%, at least 90%, or at least 100% less than diazepam

Psychological effects can be associated with drug abuse, and may constitute desired psychological effects for the user. Desired psychological effects can include one or more of euphoria, hallucinations and other perceptual distortions, alterations in cognition, and changes in mood. Desired psychological effects can also include sedative effects, stimulant effects, positive effects, and negative effects. Sedative effects include, but are not limited to, alertness, drowsiness, and sedation. Stimulant effects include, but are not limited to, stimulation. Positive effects include, but are not limited to, euphoria and drug high. Negative effects include, but are not limited to, agitation and dysphoria.

In certain embodiments of the methods provided herein, the method does not produce desired psychological effects in the subject. In some embodiments, the method does not induce desired psychological effects in the subject. In some embodiments, the subject does not experience desired psychological effects.

In some embodiments, the method does not produce hallucinations in the subject. In some embodiments, the method does not induce hallucinations in the subject. In some embodiments, the subject does not experience hallucinations. In some embodiments, administration of bitopertin induces a hallucination response comparable to placebo. In some embodiments, administration of bitopertin induces a hallucination response that is at least 40%, at least 50%, at least 60%, at least 70%, at least 80%, at least 90%, or at least 100% less than a positive control. In some embodiments, the positive control comprises diazepam. In some embodiments, administration of bitopertin induces a hallucination response that is at least 40%, at least 50%, at least 60%, at least 70%, at least 80%, at least 90%, or at least 100% less than diazepam.

In some embodiments, the method does not produce perceptual distortions in the subject. In some embodiments, the method does not induce perceptual distortions in the subject. In some embodiments, the subject does not experience perceptual distortions. In some embodiments, administration of bitopertin induces a perceptual distortion response comparable to placebo. In some embodiments, administration of bitopertin induces a perceptual distortion response that is at least 40%, at least 50%, at least 60%, at least 70%, at least 80%, at least 90%, or at least 100% less than a positive control. In some embodiments, the positive control comprises diazepam. In some embodiments, administration of bitopertin induces a perceptual distortion response that is at least 40%, at least 50%, at least 60%, at least 70%, at least 80%, at least 90%, or at least 100% less than diazepam.

In some embodiments, the method does not produce alterations in cognition in the subject. In some embodiments, the method does not induce alterations in cognition in the subject. In some embodiments, the subject does not experience alterations in cognition. In some embodiments, administration of bitopertin induces a cognitive alteration response comparable to placebo. In some embodiments, administration of bitopertin induces a cognitive alteration response that is at least 40%, at least 50%, at least 60%, at least 70%, at least 80%, at least 90%, or at least 100% less than a positive control. In some embodiments, the positive control comprises diazepam. In some embodiments, administration of bitopertin induces a cognitive alteration response that is at least 40%, at least 50%, at least 60%, at least 70%, at least 80%, at least 90%, or at least 100% less than diazepam.

In some embodiments, the method does not produce changes in mood in the subject. In some embodiments, the method does not induce changes in mood in the subject. In some embodiments, the subject does not experience changes in mood. In some embodiments, administration of bitopertin induces a mood change response comparable to placebo. In some embodiments, administration of bitopertin induces a mood change response that is at least 40%, at least 50%, at least 60%, at least 70%, at least 80%, at least 90%, or at least 100% less than a positive control. In some embodiments, the positive control comprises diazepam. In some embodiments, administration of bitopertin induces a mood change response that is at least 40%, at least 50%, at least 60%, at least 70%, at least 80%, at least 90%, or at least 100% less than diazepam

In some embodiments, the method does not produce stimulant effects in the subject. In some embodiments, the method does not induce stimulant effects in the subject. In some embodiments, the subject does not experience stimulant effects. In some embodiments, administration of bitopertin induces stimulant effects comparable to placebo. In some embodiments, administration of bitopertin induces stimulant effects that are at least 40%, at least 50%, at least 60%, at least 70%, at least 80%, at least 90%, or at least 100% less than a positive control. In some embodiments, the positive control comprises diazepam. In some embodiments, administration of bitopertin induces stimulant effects that are at least 40%, at least 50%, at least 60%, at least 70%, at least 80%, at least 90%, or at least 100% less than diazepam.

In some embodiments, the method does not produce euphoria in the subject. In some embodiments, the method does not induce euphoria in the subject. In some embodiments, the subject does not experience euphoria. In some embodiments, administration of bitopertin induces a euphoria response comparable to placebo. In some embodiments, administration of bitopertin induces a euphoria response that is at least 40%, at least 50%, at least 60%, at least 70%, at least 80%, at least 90%, or at least 100% less than a positive control. In some embodiments, the positive control comprises diazepam. In some embodiments, administration of bitopertin induces a euphoria response that is at least 40%, at least 50%, at least 60%, at least 70%, at least 80%, at least 90%, or at least 100% less than diazepam.

In some embodiments, the method does not produce a drug high in the subject. In some embodiments, the method does not induce a drug high in the subject. In some embodiments, the subject does not experience a drug high. In some embodiments, administration of bitopertin induces a drug high comparable to placebo. In some embodiments, administration of bitopertin induces a drug high that is at least 40%, at least 50%, at least 60%, at least 70%, at least 80%, at least 90%, or at least 100% less than a positive control. In some embodiments, the positive control comprises diazepam. In some embodiments, administration of bitopertin induces a drug high that is at least 40%, at least 50%, at least 60%, at least 70%, at least 80%, at least 90%, or at least 100% less than diazepam.

In some embodiments, the method does not produce stimulation in the subject. In some embodiments, the method does not induce stimulation in the subject. In some embodiments, the subject does not experience stimulation.

In some embodiments, the method does not produce drug liking in the subject. In some embodiments, the method does not induce drug liking in the subject. In some embodiments, the subject does not experience drug liking. In some embodiments, administration of bitopertin does not produce drug liking in the subject. In some embodiments, administration of bitopertin does not induce drug liking in the subject. In some embodiments, the subject does not experience drug liking following administration of bitopertin. In some embodiments, administration of bitopertin induces a drug liking response comparable to placebo. In some embodiments, administration of bitopertin induces a drug liking response that is at least 40%, at least 50%, at least 60%, at least 70%, at least 80%, at least 90%, or at least 100% less than a positive control. In some embodiments, the positive control comprises diazepam. In some embodiments, administration of bitopertin induces a drug liking response that is at least 40%, at least 50%, at least 60%, at least 70%, at least 80%, at least 90%, or at least 100% less than diazepam.

In some embodiments, the method does not produce drug wanting in the subject. In some embodiments, the method does not induce drug wanting in the subject. In some embodiments, the subject does not experience drug wanting. In some embodiments, administration of bitopertin does not produce drug wanting in the subject. In some embodiments, administration of bitopertin does not induce drug wanting in the subject. In some embodiments, the subject does not experience drug wanting following administration of bitopertin. In some embodiments, administration of bitopertin induces a drug wanting response comparable to placebo. In some embodiments, administration of bitopertin induces a drug wanting response that is at least 40%, at least 50%, at least 60%, at least 70%, at least 80%, at least 90%, or at least 100% less than a positive control. In some embodiments, the positive control comprises diazepam. In some embodiments, administration of bitopertin induces a drug wanting response that is at least 40%, at least 50%, at least 60%, at least 70%, at least 80%, at least 90%, or at least 100% less than diazepam.

In some embodiments, the method does not produce drug liking at the moment in the subject. In some embodiments, the method does not induce drug liking at the moment in the subject. In some embodiments, the subject does not experience drug liking at the moment. In some embodiments, administration of bitopertin does not produce drug liking at the moment in the subject. In some embodiments, administration of bitopertin does not induce drug liking at the moment in the subject. In some embodiments, the subject does not experience drug liking at the moment following administration of bitopertin. In some embodiments, administration of bitopertin induces a drug liking at the moment response comparable to placebo. In some embodiments, administration of bitopertin induces a drug liking at the moment response that is at least 40%, at least 50%, at least 60%, at least 70%, at least 80%, at least 90%, or at least 100% less than a positive control. In some embodiments, the positive control comprises diazepam. In some embodiments, administration of bitopertin induces a drug liking at the moment response that is at least 40%, at least 50%, at least 60%, at least 70%, at least 80%, at least 90%, or at least 100% less than diazepam.

In some embodiments, the method does not produce a desire to take the drug again in the subject. In some embodiments, the method does not induce a desire to take the drug again in the subject. In some embodiments, the subject does not experience a desire to take the drug again. In some embodiments, administration of bitopertin does not produce a desire to take the drug again in the subject. In some embodiments, administration of bitopertin does not induce a desire to take the drug again in the subject. In some embodiments, the subject does not experience a desire to take the drug again following administration of bitopertin.

In some embodiments, the method does not produce negative effects in the subject. In some embodiments, the method does not induce negative effects in the subject. In some embodiments, the subject does not experience negative effects.

In some embodiments, the method does not produce agitation in the subject. In some embodiments, the method does not induce agitation in the subject. In some embodiments, the subject does not experience agitation. In some embodiments, administration of bitopertin induces an agitation response comparable to placebo. In some embodiments, administration of bitopertin induces an agitation response that is at least 40%, at least 50%, at least 60%, at least 70%, at least 80%, at least 90%, or at least 100% less than a positive control. In some embodiments, the positive control comprises diazepam. In some embodiments, administration of bitopertin induces an agitation response that is at least 40%, at least 50%, at least 60%, at least 70%, at least 80%, at least 90%, or at least 100% less than diazepam.

In some embodiments, the method does not produce dysphoria in the subject. In some embodiments, the method does not induce dysphoria in the subject. In some embodiments, the subject does not experience dysphoria. In some embodiments, administration of bitopertin induces a dysphoria response comparable to placebo. In some embodiments, administration of bitopertin induces a dysphoria response that is at least 40%, at least 50%, at least 60%, at least 70%, at least 80%, at least 90%, or at least 100% less than a positive control. In some embodiments, the positive control comprises diazepam. In some embodiments, administration of bitopertin induces a dysphoria response that is at least 40%, at least 50%, at least 60%, at least 70%, at least 80%, at least 90%, or at least 100% less than diazepam.

In some embodiments, the method does not produce sedative effects in the subject. In some embodiments, the method does not induce sedative effects in the subject. In some embodiments, the subject does not experience sedative effects. In some embodiments, administration of bitopertin does not produce sedative effects in the subject. In some embodiments, administration of bitopertin does not induce sedative effects in the subject. In some embodiments, the subject does not experience sedative effects following administration of bitopertin. In some embodiments, administration of bitopertin induces sedative effects comparable to placebo. In some embodiments, administration of bitopertin induces sedative effects that are at least 40%, at least 50%, at least 60%, at least 70%, at least 80%, at least 90%, or at least 100% less than a positive control. In some embodiments, the positive control comprises diazepam. In some embodiments, administration of bitopertin induces sedative effects that are at least 40%, at least 50%, at least 60%, at least 70%, at least 80%, at least 90%, or at least 100% less than diazepam.

In some embodiments, the method produces a dose-dependent increase in sedative effects as measured by the PCAG subscale. In some embodiments, the method induces a dose-dependent increase in sedative effects as measured by the PCAG subscale. In some embodiments, the subject experiences a dose-dependent increase in sedative effects as measured by the PCAG subscale. In some embodiments, administration of bitopertin produces a dose-dependent increase in sedative effects as measured by the PCAG subscale. In some embodiments, administration of bitopertin induces a dose-dependent increase in sedative effects as measured by the PCAG subscale. In some embodiments, the subject does not experience a dose-dependent increase in sedative effects as measured by the PCAG subscale following administration of bitopertin. In some embodiments, administration of bitopertin induces a dose-dependent increase in sedative effects as measured by the PCAG subscale that is comparable to placebo. In some embodiments, administration of bitopertin induces a dose-dependent increase in sedative effects as measured by the PCAG subscale that is at least 40%, at least 50%, at least 60%, at least 70%, at least 80%, at least 90%, or at least 100% less than a positive control. In some embodiments, the positive control comprises diazepam. In some embodiments, administration of bitopertin induces a dose-dependent increase in sedative effects as measured by the PCAG subscale that is at least 40%, at least 50%, at least 60%, at least 70%, at least 80%, at least 90%, or at least 100% less than diazepam. In some embodiments, administration of bitopertin induces a dose-dependent increase in sedative effects without statistically significant abuse potential.

In some embodiments, the method does not produce drug liking in the subject as measured by a Visual Analogue Scale (VAS) for Drug Liking. In some embodiments, the method does not induce drug liking in the subject as measured by a VAS for Drug Liking. In some embodiments, the subject does not experience drug liking as measured by a VAS for Drug Liking. In some embodiments, administration of bitopertin does not produce drug liking in the subject as measured by a VAS for Drug Liking. In some embodiments, administration of bitopertin does not induce drug liking in the subject as measured by a VAS for Drug Liking. In some embodiments, the subject does not experience drug liking following administration of bitopertin as measured by a VAS for Drug Liking.

In some embodiments, administration of bitopertin induces a drug liking response comparable to placebo as measured by a VAS for Drug Liking. In some embodiments, administration of bitopertin induces a drug liking response that is at least 40%, at least 50%, at least 60%, at least 70%, at least 80%, at least 90%, or at least 100% less than a positive control as measured by a VAS for Drug Liking. In some embodiments, the positive control comprises diazepam. In some embodiments, administration of bitopertin induces a drug liking response that is at least 40%, at least 50%, at least 60%, at least 70%, at least 80%, at least 90%, or at least 100% less than diazepam as measured by a VAS for Drug Liking.

In some embodiments, the method does not produce overall drug liking in the subject as measured by a VAS for Overall Drug Liking. In some embodiments, the method does not induce overall drug liking in the subject as measured by a VAS for Overall Drug Liking. In some embodiments, the subject does not experience overall drug liking as measured by a VAS for Overall Drug Liking. In some embodiments, administration of bitopertin does not produce overall drug liking in the subject as measured by a VAS for Overall Drug Liking. In some embodiments, administration of bitopertin does not induce overall drug liking in the subject as measured by a VAS for Overall Drug Liking. In some embodiments, the subject does not experience overall drug liking following administration of bitopertin as measured by a VAS for Overall Drug Liking.

In some embodiments, administration of bitopertin induces an overall drug liking response comparable to placebo as measured by a VAS for Overall Drug Liking. In some embodiments, administration of bitopertin induces an overall drug liking response that is at least 40%, at least 50%, at least 60%, at least 70%, at least 80%, at least 90%, or at least 100% less than a positive control as measured by a VAS for Overall Drug Liking. In some embodiments, the positive control comprises diazepam. In some embodiments, administration of bitopertin induces an overall drug liking response that is at least 40%, at least 50%, at least 60%, at least 70%, at least 80%, at least 90%, or at least 100% less than diazepam as measured by a VAS for

### Overall Drug Liking.

In some embodiments, the method does not produce drug liking at the moment in the subject as measured by a VAS for Drug Liking at the Moment. In some embodiments, the method does not induce drug liking at the moment in the subject as measured by a VAS for Drug Liking at the Moment. In some embodiments, the subject does not experience drug liking at the moment as measured by a VAS for Drug Liking at the Moment. In some embodiments, administration of bitopertin does not produce drug liking at the moment in the subject as measured by a VAS for Drug Liking at the Moment. In some embodiments, administration of bitopertin does not induce drug liking at the moment in the subject as measured by a VAS for Drug Liking at the Moment. In some embodiments, the subject does not experience drug liking at the moment following administration of bitopertin as measured by a VAS for Drug Liking at the Moment.

In some embodiments, administration of bitopertin induces a drug liking at the moment response comparable to placebo as measured by a VAS for Drug Liking at the Moment. In some embodiments, administration of bitopertin induces a drug liking at the moment response that is at least 40%, at least 50%, at least 60%, at least 70%, at least 80%, at least 90%, or at least 100% less than a positive control as measured by a VAS for Drug Liking at the Moment. In some embodiments, the positive control comprises diazepam. In some embodiments, administration of bitopertin induces a drug liking at the moment response that is at least 40%, at least 50%, at least 60%, at least 70%, at least 80%, at least 90%, or at least 100% less than diazepam as measured by a VAS for Drug Liking at the Moment.

In some embodiments, the method does not produce a desire to take the drug again in the subject as measured by a VAS for Take Drug Again. In some embodiments, the method does not induce a desire to take the drug again in the subject as measured by a VAS for Take Drug Again.

In some embodiments, the subject does not experience a desire to take the drug again as measured by a VAS for Take Drug Again. In some embodiments, administration of bitopertin does not produce a desire to take the drug again in the subject as measured by a VAS for Take Drug Again. In some embodiments, administration of bitopertin does not induce a desire to take the drug again in the subject as measured by a VAS for Take Drug Again. In some embodiments, the subject does not experience a desire to take the drug again following administration of bitopertin as measured by a VAS for Take Drug Again.

In some embodiments, administration of bitopertin induces a Take Drug Again response comparable to placebo as measured by a VAS for Take Drug Again. In some embodiments, administration of bitopertin induces a Take Drug Again response that is at least 40%, at least 50%, at least 60%, at least 70%, at least 80%, at least 90%, or at least 100% less than a positive control as measured by a VAS for Take Drug Again. In some embodiments, the positive control comprises diazepam. In some embodiments, administration of bitopertin induces a Take Drug Again response that is at least 40%, at least 50%, at least 60%, at least 70%, at least 80%, at least 90%, or at least 100% less than diazepam as measured by a VAS for Take Drug Again.

In some embodiments, the method does not produce sedative effects in the subject as measured by an Alertness/Drowsiness VAS. In some embodiments, the method does not induce sedative effects in the subject as measured by an Alertness/Drowsiness VAS. In some embodiments, the subject does not experience sedative effects as measured by an Alertness/Drowsiness VAS. In some embodiments, administration of bitopertin does not produce sedative effects in the subject as measured by an Alertness/Drowsiness VAS. In some embodiments, administration of bitopertin does not induce sedative effects in the subject as measured by an Alertness/Drowsiness VAS. In some embodiments, the subject does not experience sedative effects following administration of bitopertin as measured by an Alertness/Drowsiness VAS.

In some embodiments, administration of bitopertin induces an alertness/drowsiness response comparable to placebo as measured by an Alertness/Drowsiness VAS. In some embodiments, administration of bitopertin induces an alertness/drowsiness response that is at least 40%, at least 50%, at least 60%, at least 70%, at least 80%, at least 90%, or at least 100% less than a positive control as measured by an Alertness/Drowsiness VAS. In some embodiments, the positive control comprises diazepam. In some embodiments, administration of bitopertin induces an alertness/drowsiness response that is at least 40%, at least 50%, at least 60%, at least 70%, at least 80%, at least 90%, or at least 100% less than diazepam as measured by an Alertness/Drowsiness VAS.

In some embodiments, the method does not produce sedative effects in the subject as measured by the Pentobarbital Chlorpromazine Alcohol Group (PCAG) subscale of the Addiction Research Center Inventory (ARCI). In some embodiments, the method does not induce sedative effects in the subject as measured by the ARCI PCAG subscale. In some embodiments, the subject does not experience sedative effects as measured by the ARCI PCAG subscale. In some embodiments, administration of bitopertin does not produce sedative effects in the subject as measured by the ARCI PCAG subscale. In some embodiments, administration of bitopertin does not induce sedative effects in the subject as measured by the ARCI PCAG subscale. In some embodiments, the subject does not experience sedative effects following administration of bitopertin as measured by the ARCI PCAG subscale.

In some embodiments, administration of bitopertin induces sedative effects comparable to placebo as measured by the ARCI PCAG subscale. In some embodiments, administration of bitopertin induces sedative effects that are at least 40%, at least 50%, at least 60%, at least 70%, at least 80%, at least 90%, or at least 100% less than a positive control as measured by the ARCI PCAG subscale. In some embodiments, the positive control comprises diazepam. In some embodiments, administration of bitopertin induces sedative effects that are at least 40%, at least 50%, at least 60%, at least 70%, at least 80%, at least 90%, or at least 100% less than diazepam as measured by the ARCI PCAG subscale.

In some embodiments, the method does not produce stimulant effects in the subject as measured by the Benzedrine Group (BG) subscale of the ARCI. In some embodiments, the method does not induce stimulant effects in the subject as measured by the ARCI BG subscale. In some embodiments, the subject does not experience stimulant effects as measured by the ARCI BG subscale. In some embodiments, administration of bitopertin does not produce stimulant effects in the subject as measured by the ARCI BG subscale. In some embodiments, administration of bitopertin does not induce stimulant effects in the subject as measured by the ARCI BG subscale. In some embodiments, the subject does not experience stimulant effects following administration of bitopertin as measured by the ARCI BG subscale.

In some embodiments, administration of bitopertin induces a stimulant effects response comparable to placebo as measured by the ARCI BG subscale. In some embodiments, administration of bitopertin induces stimulant effects that are at least 40%, at least 50%, at least 60%, at least 70%, at least 80%, at least 90%, or at least 100% less than a positive control as measured by the ARCI BG subscale. In some embodiments, the positive control comprises diazepam. In some embodiments, administration of bitopertin induces stimulant effects that are at least 40%, at least 50%, at least 60%, at least 70%, at least 80%, at least 90%, or at least 100% less than diazepam as measured by the ARCI BG subscale.

In some embodiments, the method does not produce stimulant effects in the subject as measured by the Amphetamine (A) subscale of the ARCI. In some embodiments, the method does not induce stimulant effects in the subject as measured by the ARCI A subscale. In some embodiments, the subject does not experience stimulant effects as measured by the ARCI A subscale. In some embodiments, administration of bitopertin does not produce stimulant effects in the subject as measured by the ARCI A subscale. In some embodiments, administration of bitopertin does not induce stimulant effects in the subject as measured by the ARCI A subscale. In some embodiments, the subject does not experience stimulant effects following administration of bitopertin as measured by the ARCI A subscale.

In some embodiments, administration of bitopertin induces stimulant effects comparable to placebo as measured by the ARCI A subscale. In some embodiments, administration of bitopertin induces stimulant effects that are at least 40%, at least 50%, at least 60%, at least 70%, at least 80%, at least 90%, or at least 100% less than a positive control as measured by the ARCI A subscale. In some embodiments, the positive control comprises diazepam. In some embodiments, administration of bitopertin induces stimulant effects that are at least 40%, at least 50%, at least 60%, at least 70%, at least 80%, at least 90%, or at least 100% less than diazepam as measured by the ARCI A subscale.

In some embodiments, the method does not produce a drug high in the subject as measured by a Drug High VAS. In some embodiments, the method does not induce a drug high in the subject as measured by a Drug High VAS. In some embodiments, the subject does not experience a drug high as measured by a Drug High VAS. In some embodiments, administration of bitopertin does not produce a drug high in the subject as measured by a Drug High VAS. In some embodiments, administration of bitopertin does not induce a drug high in the subject as measured by a Drug High VAS. In some embodiments, the subject does not experience a drug high following administration of bitopertin as measured by a Drug High VAS.

In some embodiments, administration of bitopertin induces a drug high response comparable to placebo as measured by a Drug High VAS. In some embodiments, administration of bitopertin induces a drug high response that is at least 40%, at least 50%, at least 60%, at least 70%, at least 80%, at least 90%, or at least 100% less than a positive control as measured by a Drug High VAS. In some embodiments, the positive control comprises diazepam. In some embodiments, administration of bitopertin induces a drug high response that is at least 40%, at least 50%, at least 60%, at least 70%, at least 80%, at least 90%, or at least 100% less than diazepam as measured by a Drug High VAS.

In some embodiments, the method does not produce positive effects in the subject as measured by the Morphine Benzedrine Group (MBG) subscale of the ARCI. In some embodiments, the method does not induce positive effects in the subject as measured by the ARCI MBG subscale. In some embodiments, the subject does not experience positive effects as measured by the ARCI MBG subscale. In some embodiments, administration of bitopertin does not produce positive effects in the subject as measured by the ARCI MBG subscale. In some embodiments, administration of bitopertin does not induce positive effects in the subject as measured by the ARCI MBG subscale. In some embodiments, the subject does not experience positive effects following administration of bitopertin as measured by the ARCI MBG subscale.

In some embodiments, administration of bitopertin induces a positive effects response comparable to placebo as measured by the ARCI MBG subscale. In some embodiments, administration of bitopertin induces a positive effects response that is at least 40%, at least 50%, at least 60%, at least 70%, at least 80%, at least 90%, or at least 100% less than a positive control as measured by the ARCI MBG subscale. In some embodiments, the positive control comprises diazepam. In some embodiments, administration of bitopertin induces positive effects that are at least 40%, at least 50%, at least 60%, at least 70%, at least 80%, at least 90%, or at least 100% less than diazepam as measured by the ARCI MBG subscale.

In some embodiments, the method does not produce negative effects in the subject as measured by the Lysergic Acid Diethylamide (LSD) subscale of the ARCI. In some embodiments, the method does not induce negative effects in the subject as measured by the ARCI LSD subscale. In some embodiments, the subject does not experience negative effects as measured by the ARCI LSD subscale. In some embodiments, administration of bitopertin does not produce negative effects in the subject as measured by the ARCI LSD subscale. In some embodiments, administration of bitopertin does not induce negative effects in the subject as measured by the ARCI LSD subscale. In some embodiments, the subject does not experience negative effects following administration of bitopertin as measured by the ARCI LSD subscale.

In some embodiments, administration of bitopertin induces a negative effects response comparable to placebo as measured by the ARCI LSD subscale. In some embodiments, administration of bitopertin induces a negative effects response that is at least 40%, at least 50%, at least 60%, at least 70%, at least 80%, at least 90%, or at least 100% less than a positive control as measured by the ARCI LSD subscale. In some embodiments, the positive control comprises diazepam. In some embodiments, administration of bitopertin induces a negative effects response that is at least 40%, at least 50%, at least 60%, at least 70%, at least 80%, at least 90%, or at least 100% less than diazepam as measured by the ARCI LSD subscale.

Porphyrins (e.g., PPIX or ZPPIX) can be found in various biological samples including the skin, urine, stool, plasma, and erythrocytes. In some embodiments, the porphyrins may be extracted from the biological sample into a solution for fluorescence analysis. Porphyrins can be detected in these biological samples by direct inspection using long wavelength ultraviolet light (e.g., 400-420 nm light). Porphyrins have the greatest absorption wavelengths near 400-420 nm, with their highest absorption peak occurring at 415 nm. The emission maxima of porphyrins is typically around 600 nm and varies slightly based on the type of porphyrins and the solvent used for analysis. In some embodiments, diagnosis of EPP or XLPP may be made using fluorescence analysis. In some embodiments, skin porphyrin levels (e.g., PPIX levels) can be measured by calculating the difference before and after complete photobleaching of PPIX using controlled illumination. See, e.g., Heerfordt I M. Br J Dermatol. 2016; 175 (6): 1284-1289.

In some embodiments of the methods provided herein, administering bitopertin, e.g., 60 mg of bitopertin, to a subject in need thereof results in a reduction of PPIX levels. In some embodiments, administration of bitopertin results in a 20% or more reduction in PPIX levels. In some embodiments, administration of bitopertin results in a 30% or more reduction in PPIX levels. In some embodiments, administration of bitopertin results in a 40% or more′ reduction in PPIX levels. In some embodiments, administration of bitopertin results in a 50% or more reduction in PPIX levels. In some embodiments, administration of bitopertin results in a 60% or more reduction in PPIX levels. In some embodiments, administration of bitopertin results in a 70% or more reduction in PPIX levels. In some embodiments, administration of bitopertin results in an 80% or more reduction in PPIX levels. In some embodiments, administration of bitopertin results in a 90% or more reduction in PPIX levels. In some embodiments, administration of bitopertin results in a 100% or more reduction in PPIX levels. In some embodiments, administration of bitopertin does not result in statistically significant abuse potential.

In some embodiments, the subject's plasma porphyrin fluoresces at a peak of 634 nm when illuminated with blue light (e.g., 400-420 nm light). In some embodiments, the subject's plasma porphyrin fluoresces at a peak between 626 nm and 634 nm when illuminated with blue light (e.g., 400-420 nm light). In some embodiments, the subject's skin porphyrin fluoresces at a peak of 632 nm when illuminated with blue light (e.g., 400-420 nm light). In some embodiments, the subject's skin porphyrin fluoresces at a peak between 626 nm and 634 nm when illuminated with blue light (e.g., 400-420 nm light). In some embodiments, the subject has greater than 0.2 FluoDerm Units (FDU) of protoporphyrin IX levels in the skin. In some embodiments, the subject has greater than 1.0 FDU of protoporphyrin IX levels in the skin. In some embodiments, the subject has between 1.0 FDU and 2.5 FDU of protoporphyrin IX levels in the skin. In some embodiments, the subject has greater than 2.5 FDU of protoporphyrin IX levels in the skin. In some embodiments, the method decreases protoporphyrin IX levels in the skin of the subject to less than 0.5 FDU. In some embodiments, the method decreases protoporphyrin IX levels in the skin of the subject to less than 1.0 FDU. In some embodiments, the method decreases protoporphyrin IX levels in the skin of the subject to less than 1.5 FDU. In some embodiments, the method decreases protoporphyrin IX levels in the skin of the subject to less than 2.0 FDU. In some embodiments, the method decreases protoporphyrin IX levels in the skin of the subject to less than 2.5 FDU. In some embodiments, the subject has red fluorescent urine. In some embodiments, the subject has a peak between 615 nm and 620 nm using plasma porphyrin fluorescence analysis. In some preferred embodiments, the subject does not experience changes in skin pigmentation. In some embodiments, the method does not produce statistically significant abuse potential. In some embodiments, the method does not induce statistically significant abuse potential. In some embodiments, the method does not result in statistically significant abuse potential.

In certain aspects, the disclosure relates to methods of preventing, treating, or reducing the progression rate and/or severity of one or more complications of EPP or XLPP in a subject, the method comprising administering to the subject a pharmaceutical composition comprising bitopertin. In some embodiments, the one or more complications of EPP or XLPP is selected from the group consisting of: acute photosensitivity, cutaneous photosensitivity, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholelithiasis, cholestasis, cytolysis, gallstones, cholestatic liver failure, erythrodontia, hypercellular bone marrow, myelodysplasia, thrombocytopenia, hydrops fetalis and/or death in utero. In some embodiments, the disclosure contemplates methods of treating one or more complications of EPP or XLPP (e.g., acute photosensitivity, cutaneous photosensitivity, edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholelithiasis, cholestasis, cytolysis, gallstones, cholestatic liver failure, erythrodontia, hypercellular bone marrow, myelodysplasia, thrombocytopenia, hydrops fetalis and/or death in utero) comprising administering to the subject a pharmaceutical composition comprising bitopertin. In some embodiments, the one or more complications are improved indirectly. In some embodiments, the disclosure contemplates methods of preventing one or more complications of EPP or XLPP comprising administering to the subject a pharmaceutical composition comprising bitopertin. In some embodiments, the disclosure contemplates methods of reducing the progression rate of one or more complications of EPP or XLPP comprising administering to the subject a pharmaceutical composition comprising bitopertin. In some embodiments, the disclosure contemplates methods of reducing the severity of one or more complications of EPP or XLPP comprising administering to the subject a pharmaceutical composition comprising bitopertin. In some embodiments, administration of bitopertin does not produce statistically significant abuse potential. In some embodiments, administration of bitopertin does not induce statistically significant abuse potential. In some embodiments, administration of bitopertin does not result in statistically significant abuse potential.

Optionally, methods disclosed herein for preventing, treating, or reducing the progression rate and/or severity of one or more complications of EPP or XLPP in a subject, may further comprise administering to the patient one or more supportive therapies or additional active agents for treating EPP or XLPP. For example, the patient also may be administered one or more supportive therapies or active agents selected from the group consisting of: avoiding sunlight, topical sunscreens, skin protection, UVB phototherapy, Afamelanotide (Scenesse®), bortezomib, proteasome inhibitors, chemical chaperones, cholestyramine, activated charcoal, iron supplementation, liver transplantation, bone marrow transplantation, splenectomy, and blood transfusion. In some embodiments, the methods described herein may further comprise administering to the patient Afamelanotide (Scenesse®).

Certain adverse events or adverse reactions present in subjects treated with medical interventions, e.g., bitopertin. Such adverse events or adverse reactions can manifest as Central Nervous System (CNS) adverse events, or adverse reactions. CNS adverse events can affect the brain and spinal cord, and can manifest in a variety of symptoms, including, but not limited to drowsiness, dizziness, impaired concentration, vertigo, headaches, nausea, insomnia, ataxia, and/or coma. Further adverse events or adverse reactions can manifest as anemia adverse events or adverse reactions. Anemia adverse events or adverse reactions are caused due to a lack of sufficient red blood cells and include, but are not limited to shortness of breath, difficulty breathing, weakness, fatigue, dizziness, lightheadedness, headaches, nausea, irregular heartbeat, chest pain, tinnitus, swelling, and/or jaundice. In some embodiments of the methods provided herein, the subject experiences less than 20%, less than 15%, less than 10%, less than 9%, less than 8%, less than 7%, less than 6%, less than 5%, less than 4%, less than 3%, less than 2%, or less than 1% of adverse events associated with the central nervous system (CNS). In some embodiments of the methods provided herein, the subject experiences less than 15% of adverse events associated with the central nervous system (CNS). In some embodiments of the methods provided herein, the subject experiences less than 10% of adverse events associated with the central nervous system (CNS). In some embodiments of the methods provided herein, the subject experiences less than 5% of adverse events associated with the central nervous system (CNS). In some embodiments of the methods provided herein, the subject experiences less than 5% of adverse events associated with anemia. In some embodiments, the adverse events associated with the CNS are selected from drowsiness, dizziness, impaired concentration, vertigo, headaches, nausea, insomnia, ataxia, and/or coma. In some embodiments, the adverse events associated with anemia are selected from shortness of breath, difficulty breathing, weakness, fatigue, dizziness, lightheadedness, headaches, nausea, irregular heartbeat, chest pain, tinnitus, swelling, and/or jaundice. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

Porphyrin photosensitization in EPP and XLPP produces two distinct clinical syndromes: (1) acute photosensitivity on exposure to sunlight with erythema and edema and (2) a syndrome wherein subepidermal bullae occur in sun-exposed areas of the skin. Photosensitization in EPP thereby produces painful phototoxic reactions upon exposure to light. Light as used herein is given its usual and ordinary meaning, and includes sunlight, visible light, and ultraviolet (UV) radiation (e.g., UVA radiation and UVB radiation). Following exposure to light, the time period before a subject with, e.g., EPP, experiences pain due to the light exposure is referenced herein as pain-free light exposure, time of pain-free light exposure, pain-free time in light, or light sensitivity.

In certain aspects, the disclosure relates to methods of preventing, treating, or reducing the progression rate and/or severity of EPP or XLPP in a subject, the method comprising administering to the subject a pharmaceutical composition comprising bitopertin, wherein the method increases pain free light exposure in the subject. In some embodiments, the method increases pain free light exposure in the subject by at least 10%, 20%, 30%, 40%, or 50% more as compared to pain free light exposure prior to administration of the bitopertin. In some embodiments, the method decreases light sensitivity in the subject. In some embodiments, the method decreases light sensitivity in the subject by at least 10%, 20%, 30%, 40%, or 50% more as compared to light sensitivity prior to administration of the bitopertin. In some embodiments, the subject has a history of phototoxic reactions from EPP. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some preferred embodiments of the methods provided herein, administering 60 mg of bitopertin to a subject in need thereof for any treatment period provided herein thereby results in an improvement in sunlight tolerance. In some embodiments, the improvement in sunlight tolerance is statistically significant. In some embodiments, the improvement in sunlight tolerance occurs after at least 1 week of treatment. In some embodiments, the improvement in sunlight tolerance occurs after at least 2 weeks of treatment. In some embodiments, the improvement in sunlight tolerance occurs after at least 3 weeks of treatment. In some embodiments, the improvement in sunlight tolerance occurs after at least 4 weeks of treatment. In some embodiments, the improvement in sunlight tolerance occurs after at least 5 weeks of treatment. In some embodiments, the improvement in sunlight tolerance occurs after at least 6 weeks of treatment. In some embodiments, the improvement in sunlight tolerance occurs after at least 7 weeks of treatment. In some embodiments, the improvement in sunlight tolerance occurs after at least 8 weeks of treatment. In some embodiments, the improvement in sunlight tolerance occurs after at least 9 weeks of treatment. In some embodiments, the improvement in sunlight tolerance occurs after at least 10 weeks of treatment. In some embodiments, the improvement in sunlight tolerance occurs after at least 11 weeks of treatment. In some embodiments, the improvement in sunlight tolerance occurs after at least 12 weeks of treatment. In some embodiments, the improvement in sunlight tolerance occurs after at least 13 weeks of treatment. In some embodiments, the improvement in sunlight tolerance occurs after at least 14 weeks of treatment. In some embodiments, the improvement in sunlight tolerance occurs after at least 15 weeks of treatment. In some embodiments, the improvement in sunlight tolerance occurs after at least 16 weeks of treatment. In some embodiments, the improvement in sunlight tolerance occurs after at least 17 weeks of treatment. In some embodiments, the improvement in sunlight tolerance occurs after at least 18 weeks of treatment. In some embodiments, the improvement in sunlight tolerance occurs after at least 19 weeks of treatment. In some embodiments, the improvement in sunlight tolerance occurs after at least 20 weeks of treatment. In some embodiments, the sunlight tolerance of the subject increases as compared to the sunlight tolerance prior to treatment. In some preferred embodiments, the method comprises administering to the subject 60 mg of bitopertin. In some embodiments, the method comprises a treatment period of at least 17 weeks. In some embodiments, the method results in a statistically significant increase in sunlight tolerance without statistically significant abuse potential. In some embodiments, the subject has EPP. In some embodiments, the subject has XLPP.

In some preferred embodiments of the methods provided herein, administering 60 mg of bitopertin to a subject in need thereof for any treatment period provided herein thereby results in a statistically significant improvement in pain-free light exposure in the subject. In some embodiments, the improvement in pain-free light exposure occurs after at least 1 week of treatment. In some embodiments, the improvement in pain-free light exposure occurs after at least 2 weeks of treatment. In some embodiments, the improvement in pain-free light exposure occurs after at least 3 weeks of treatment. In some embodiments, the improvement in pain-free light exposure occurs after at least 4 weeks of treatment. In some embodiments, the improvement in pain-free light exposure occurs after at least 5 weeks of treatment. In some embodiments, the improvement in pain-free light exposure occurs after at least 6 weeks of treatment. In some embodiments, the improvement in pain-free light exposure occurs after at least 7 weeks of treatment. In some embodiments, the improvement in pain-free light exposure occurs after at least 8 weeks of treatment. In some embodiments, the improvement in pain-free light exposure occurs after at least 9 weeks of treatment. In some embodiments, the improvement in pain-free light exposure occurs after at least 10 weeks of treatment. In some embodiments, the improvement in pain-free light exposure occurs after at least 11 weeks of treatment. In some embodiments, the improvement in pain-free light exposure occurs after at least 12 weeks of treatment. In some embodiments, the improvement in pain-free light exposure occurs after at least 13 weeks of treatment. In some embodiments, the improvement in pain-free light exposure occurs after at least 14 weeks of treatment. In some embodiments, the improvement in pain-free light exposure occurs after at least 15 weeks of treatment. In some embodiments, the improvement in pain-free light exposure occurs after at least 16 weeks of treatment. In some embodiments, the improvement in pain-free light exposure occurs after at least 17 weeks of treatment. In some embodiments, the improvement in pain-free light exposure occurs after at least 18 weeks of treatment. In some embodiments, the improvement in pain-free light exposure occurs after at least 19 weeks of treatment. In some embodiments, the improvement in pain-free light exposure occurs after at least 20 weeks of treatment. In some embodiments, the total pain-free time in light of the subject increases as compared to the total pain-free time in light prior to treatment.

In some embodiments, the improvement in pain-free light exposure is sustained for at least 1 week. In some embodiment, the improvement in pain-free light exposure is sustained for at least 2 weeks. n some embodiment, the improvement in pain-free light exposure is sustained for at least 3 weeks. In some embodiment, the improvement in pain-free light exposure is sustained for at least 4 weeks. In some embodiment, the improvement in pain-free light exposure is sustained for at least 5 weeks. In some embodiment, the improvement in pain-free light exposure is sustained for at least 6 weeks. In some embodiment, the improvement in pain-free light exposure is sustained for at least 7 weeks. In some embodiment, the improvement in pain-free light exposure is sustained for at least 8 weeks. In some embodiment, the improvement in pain-free light exposure is sustained for at least 9 weeks. In some embodiment, the improvement in pain-free light exposure is sustained for at least 10 weeks. In some embodiment, the improvement in pain-free light exposure is sustained for at least 11 weeks. In some embodiment, the improvement in pain-free light exposure is sustained for at least 12 weeks. In some embodiment, the improvement in pain-free light exposure is sustained for at least 13 weeks. In some embodiment, the improvement in pain-free light exposure is sustained for at least 14 weeks. In some embodiment, the improvement in pain-free light exposure is sustained for at least 15 weeks. In some embodiment, the improvement in pain-free light exposure is sustained for at least 16 weeks. In some embodiment, the improvement in pain-free light exposure is sustained for at least 17 weeks. In some embodiment, the improvement in pain-free light exposure is sustained for at least 18 weeks. In some embodiment, the improvement in pain-free light exposure is sustained for at least 19 weeks. In some embodiment, the improvement in pain-free light exposure is sustained for at least 20 weeks. In some embodiment, the improvement in pain-free light exposure is sustained for at least 30 weeks. In some embodiment, the improvement in pain-free light exposure is sustained for at least 40 weeks. In some embodiment, the improvement in pain-free light exposure is sustained for at least 50 weeks. In some embodiment, the improvement in pain-free light exposure is sustained for at least 52 weeks.

In some embodiments of the methods provided herein, administering 60 mg of bitopertin to a subject in need thereof for any treatment period provided herein thereby results in a statistically significant improvement in pain-free light exposure in the subject. In some embodiments, the average daily time of pain-free light exposure is increased by at least 10%. In some embodiments, the average daily time of pain-free light exposure is increased by at least 20%. In some embodiments, the average daily time of pain-free light exposure is increased by at least 30%. In some embodiments, the average daily time of pain-free light exposure is increased by at least 40%. In some embodiments, the average daily time of pain-free light exposure is increased by at least 50%. In some embodiments, the average daily time of pain-free light exposure is increased by at least 55%. In some embodiments, the average daily time of pain-free light exposure is increased by at least 60%. In some embodiments, the average daily time of pain-free light exposure is increased by at least 65%. In some embodiments, the average daily time of pain-free light exposure is increased by at least 70%. In some embodiments, the average daily time of pain-free light exposure is increased by at least 75%. In some embodiments, the average daily time of pain-free light exposure is increased by at least 80%. In some embodiments, the average daily time of pain-free light exposure is increased by at least 85%. In some embodiments, the average daily time of pain-free light exposure is increased by at least 90%. In some embodiments, the average daily time of pain-free light exposure is increased by at least 95%. In some embodiments, the average daily time of pain-free light exposure is increased by at least 100%. In some embodiments, the average daily time of pain-free light exposure is increased by at least 2-fold. In some embodiments, the average daily time of pain-free light exposure is increased by at least 3-fold. In some embodiments, the average daily time of pain-free light exposure is increased by at least 4-fold. In some embodiments, the average daily time of pain-free light exposure is increased by at least 5-fold. In a further embodiment, the treatment period is 17 weeks.

In some preferred embodiments of the methods provided herein, administering 60 mg of bitopertin to a subject in need thereof for a treatment period of at least 17 weeks thereby results in a statistically significant improvement in pain-free light exposure in the subject. In some embodiments, the average daily time of pain-free light exposure is increased by at least 1 hour. In some embodiments, the average daily time of pain-free light exposure is increased by at least 2 hours. In some embodiments, the average daily time of pain-free light exposure is increased by at least 3 hours. In some embodiments, the average daily time of pain-free light exposure is increased by at least 4 hours. In some embodiments, the average daily time of pain-free light exposure is increased by at least 5 hours. In some embodiments, the average daily time of pain-free light exposure is increased by at least 6 hours. In some embodiments, the average daily time of pain-free light exposure is increased by at least 7 hours. In some embodiments, the average daily time of pain-free light exposure is increased by at least 8 hours. In some embodiments, the average daily time of pain-free light exposure is increased by at least 9 hours. In some embodiments, the average daily time of pain-free light exposure is increased by at least 10 hours. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some further embodiments of the methods provided herein, the subject's pain free light exposure is increased to 1 or more hours compared to observed pain free light exposure in treatment with placebo. In some embodiments, the subject's pain free light exposure is increased to 2 or more hours compared to observed pain free light exposure in treatment with placebo. the subject's pain free light exposure is increased to 3 or more hours compared to observed pain free light exposure in treatment with placebo. In some embodiments, the subject's pain free light exposure is increased to 4 or more hours compared to observed pain free light exposure in treatment with placebo. In some embodiment, the subject's pain free light exposure is increased to 5 or more hours compared to observed pain free light exposure in treatment with placebo. In some embodiments, the subject's pain free light exposure is increased to 6 or more hours compared to observed pain free light exposure in treatment with placebo. In some embodiments, the subject's pain free light exposure is increased to 7 or more hours compared to observed pain free light exposure in treatment with placebo. In some embodiments, the subject's pain free light exposure is increased to 8 or more hours compared to observed pain free light exposure in treatment with placebo. In some embodiments, the subject's pain free light exposure is increased to 9 or more hours compared to observed pain free light exposure in treatment with placebo. In some embodiments, the subject's pain free light exposure is increased to 10 or more hours compared to observed pain free light exposure in treatment with placebo.

In some preferred embodiments of the methods provided herein, administering 60 mg of bitopertin to a subject in need thereof for a treatment period of at least 17 weeks thereby results in a statistically significant improvement in pain-free time in light in the subject. In some embodiments, the average daily time of pain-free time in light is increased by at least 1 hour. In some embodiments, the average daily time of pain-free time in light is increased by at least 2 hours. In some embodiments, the average daily time of pain-free time in light is increased by at least 3 hours. In some embodiments, the average daily time of pain-free time in light is increased by at least 4 hours. In some embodiments, the average daily time of pain-free time in light is increased by at least 5 hours. In some embodiments, the average daily time of pain-free time in light is increased by at least 6 hours. In some embodiments, the average daily time of pain-free time in light is increased by at least 7 hours. In some embodiments, the average daily time of pain-free time in light is increased by at least 8 hours. In some embodiments, the average daily time of pain-free time in light is increased by at least 9 hours. In some embodiments, the average daily time of pain-free time in light is increased by at least 10 hours.

In some further embodiments of the methods provided herein, the subject's pain-free time in light is increased to 1 or more hours compared to observed pain-free time in light in treatment with placebo. In some embodiments, the subject's pain-free time in light is increased to 2 or more hours compared to observed pain-free time in light in treatment with placebo, the subject's pain-free time in light is increased to 3 or more hours compared to observed pain-free time in light in treatment with placebo. In some embodiments, the subject's pain-free time in light is increased to 4 or more hours compared to observed pain-free time in light in treatment with placebo. In some embodiment, the subject's pain-free time in light is increased to 5 or more hours compared to observed pain-free time in light in treatment with placebo. In some embodiments, the subject's pain-free time in light is increased to 6 or more hours compared to observed pain-free time in light in treatment with placebo. In some embodiments, the subject's pain-free time in light is increased to 7 or more hours compared to observed pain-free time in light in treatment with placebo. In some embodiments, the subject's pain-free time in light is increased to 8 or more hours compared to observed pain-free time in light in treatment with placebo. In some embodiments, the subject's pain-free time in light is increased to 9 or more hours compared to observed pain-free time in light in treatment with placebo. In some embodiments, the subject's pain-free time in light is increased to 10 or more hours compared to observed pain-free time in light in treatment with placebo.

Phototoxic reaction is an adverse skin reaction that occurs when certain substances, called photosensitizers, interact with ultraviolet (UV) light from the sun or artificial sources. The phototoxic reaction in EPP/XLPP is a consequence of high levels of metal free protoporphyrin in the blood. When exposed to light and especially blue light in the visible light range, metal free protoporphyrin is activated to form oxygen radicals, which in turn cause a phototoxic reaction.

In certain embodiments of the present disclosure, the methods provided herein comprise administering bitopertin to a subject in need thereof for any treatment period provided herein, thereby reducing the rate of phototoxic reactions compared to the rate observed in treatment with placebo. As used herein, the term “placebo” refers to any substance or treatment that appears to be a medical intervention but contains no active ingredients. In some embodiments, the rate of phototoxic reactions compared to the rate observed in treatment with placebo is reduced by 40% or more. In some embodiments, the rate of phototoxic reactions compared to the rate observed in treatment with placebo is reduced by 50% or more. In some embodiments, the rate of phototoxic reactions compared to the rate observed in treatment with placebo is reduced by 60% or more. In some embodiments, the rate of phototoxic reactions compared to the rate observed in treatment with placebo is reduced by 70% or more. In some embodiments, the rate of phototoxic reactions compared to the rate observed in treatment with placebo is reduced by 75% or more. In some embodiments, the rate of phototoxic reactions compared to the rate observed in treatment with placebo is reduced by 80% or more. In some embodiments, the rate of phototoxic reactions compared to the rate observed in treatment with placebo is reduced by 85% or more. In some embodiments, the rate of phototoxic reactions compared to the rate observed in treatment with placebo is reduced by 90% or more. In some embodiments, the rate of phototoxic reactions compared to the rate observed in treatment with placebo is reduced by 95% or more. In some embodiments, the rate of phototoxic reactions compared to the rate observed in treatment with placebo is reduced by 100%. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some preferred embodiments, the methods comprise administering 60 mg of bitopertin to a subject in need thereof for a treatment period of at least 17 weeks, thereby reducing the rate of phototoxic reactions compared to the rate observed in treatment with placebo by 60% or more. In some preferred embodiments, the methods comprise administering 60 mg of bitopertin to a subject in need thereof for a treatment period of at least 17 weeks, thereby reducing the rate of phototoxic reactions compared to the rate observed in treatment with placebo by 70% or more. In some preferred embodiments, the methods comprise administering 60 mg of bitopertin to a subject in need thereof for a treatment period of at least 17 weeks, thereby reducing the rate of phototoxic reactions compared to the rate observed in treatment with placebo by 75% or more.

Subjects with EPP or XLPP also experience prodromal sensations upon exposure to light. For subjects with EPP or XLPP, these sensations serve as signals to seek shade and/or move away from light to avoid impending phototoxic reaction. Prodromal sensations include, but are not limited to burning, tingling, itching, and/or stinging. The time required for prodromal sensations to occur is referenced herein as “time to prodrome”. Prodrome-free light exposure, as used herein, refers to light exposure without onset prodromal symptoms or sensations, e.g., burning, tingling, itching, and/or stinging. In some embodiments of the methods provided herein, administering 60 mg of bitopertin to a subject in need thereof for any treatment period provided herein results in an increase in the average time to prodrome. In some embodiments, the increase in the average time to prodrome is at least 30 minutes from baseline. In some embodiments, the increase in the average time to prodrome is at least 40 minutes from baseline. In some embodiments, the increase in the average time to prodrome is at least 45 minutes from baseline. In some embodiments, the increase in the average time to prodrome is at least 50 minutes from baseline. In some embodiments, the increase in the average time to prodrome is at least 55 minutes from baseline. In some embodiments, the increase in the average time to prodrome is at least 60 minutes from baseline. In some embodiments, the increase in the average time to prodrome is at least 70 minutes from baseline. In some embodiments, the increase in the average time to prodrome is at least 80 minutes from baseline. In some embodiments, the increase in the average time to prodrome is at least 90 minutes from baseline. In some embodiments, the increase in the average time to prodrome is at least 100 minutes from baseline. In some embodiments, the increase in the average time to prodrome is at least 110 minutes from baseline. In some embodiments, the increase in the average time to prodrome is at least 115 minutes from baseline. In some embodiments, the increase in the average time to prodrome is at least 120 minutes from baseline. In some further preferred embodiments, the treatment period is 17-weeks. In some further preferred embodiments, the subject is age 12 and older.

In some embodiments of the methods provided herein, administering 60 mg of bitopertin to a subject in need thereof for any treatment period provided herein results in an increase in the proportion of prodrome-free weekly light exposure challenges. In some embodiments, the increase in the proportion of prodrome free weekly light exposure challenges is 25%. In some embodiments, the increase in the proportion of prodrome free weekly light exposure challenges is 40%. In some embodiments, the increase in the proportion of prodrome free weekly light exposure challenges is 50%. In some embodiments, the increase in the proportion of prodrome free weekly light exposure challenges is 55%. In some embodiments, the increase in the proportion of prodrome free weekly light exposure challenges is 65%. In some embodiments, the increase in the proportion of prodrome free weekly light exposure challenges is 75%. In some embodiments, the increase in the proportion of prodrome free weekly light exposure challenges is 85%. In some embodiments, the increase in the proportion of prodrome free weekly light exposure challenges is 95%. In some embodiments, the increase in the proportion of prodrome free weekly light exposure challenges is 100%. In some further preferred embodiments, the treatment period is 17-weeks. In some further preferred embodiments, the subject is age 12 and older.

Pain associated with phototoxic reaction may be scored utilizing a pain score due to phototoxic reactions, as assessed on a numerical rating scale of 1 (least) to 10 (worst) and compared to placebo. In some embodiments of the methods provided herein, administering 60 mg of bitopertin to a subject in need thereof for any treatment period provided herein results in a reduction in a median maximum pain score due to phototoxic reactions over the treatment period. In some embodiments, the median maximum pain score due to phototoxic reactions is reduced by at least 1 unit on a pain score rating scale over the treatment period. In some further preferred embodiments, the treatment period is 17-weeks. In some further preferred embodiments, the subject is age 12 and older.

A variety of questionnaires are utilized to capture Patient reported outcomes (PROs) and assess health related quality of life impacts of clinical therapies, e.g., bitopertin, in EPP and XLPP. The EPP Impact Questionnaire (EPIQ) is a Sponsor-developed exploratory tool that includes quality of life (QOL) measures. The EPIQ includes questions related to reactions to light, symptoms of such reactions, the subject's ability to do things as impacted by EPP or XLPP, and their overall quality of life compared to someone without EPP. In some embodiments of the methods provided herein, administering 60 mg of bitopertin to a subject in need thereof for any treatment period provided herein results in an improvement in the EPP Impact Questionnaire (EPIQ). In some further preferred embodiments, the treatment period is 17-weeks. In some further preferred embodiments, the subject is age 12 and older.

The patient global impression of change (PGIC) is a scoring measure used to assess a subject's belief about the efficacy of treatment as it relates to pain. PGIC is a multi-point scale depicting a patient's rating of overall improvement. Patients rate their change as “very much improved,” “much improved,” “minimally improved,” “no change,” “minimally worse,” “much worse,” or “very much worse.” Subjects may also rate their change as “much better”, “a little better”, or “no change/a little worse”. In some embodiments of the methods provided herein, administering 60 mg of bitopertin to a subject in need thereof for any treatment period provided herein results in an improvement in the subject's the Patient Global Impression of Pain (PGIC). In some further preferred embodiments, the treatment period is 17-weeks. In some further preferred embodiments, the subject is age 12 and older.

The patient global impression of severity (PGIS) is a global index used to rate the severity of disease, e.g., EPP or XLPP. A PGIS may ask subjects, “Overall, how severe was your EPP in the past 7 days?” and provide one of the following responses, not at all, mild, moderate, severe, very severe. In some embodiments of the methods provided herein, administering 60 mg of bitopertin to a subject in need thereof for any treatment period provided herein results in an improvement in the subject's the Patient Global Impression of Severity (PGIS). In some further preferred embodiments, the treatment period is 17-weeks. In some further preferred embodiments, the subject is age 12 and older.

Glycine is one of the key initial substrates for heme and globin synthesis. As such, decreased levels of glycine due to GlyT1 inhibition could lead to a decrease in heme synthesis. In certain aspects, the disclosure relates to methods of treating EPP or XLPP in a subject, the method comprising administering to the subject a pharmaceutical composition comprising bitopertin, wherein the subject's heme levels decrease no more than 10% (e.g., 10%, 15%, 20%, 25%, and 30%). In some embodiments, the disclosure relates to methods of treating EPP or XLPP in a subject, wherein the subject's heme levels decrease no more than 15%. In some embodiments, the disclosure relates to methods of treating EPP or XLPP in a subject, wherein the subject's heme levels decrease no more than 20%. In some embodiments, the disclosure relates to methods of treating EPP or XLPP in a subject, wherein the subject's heme levels decrease no more than 25%. In some embodiments, the disclosure relates to methods of treating EPP or XLPP in a subject, wherein the subject's heme levels decrease no more than 30%.

In certain aspects, the disclosure relates to methods of treating EPP or XLPP in a subject, the method comprising administering to the subject a pharmaceutical composition comprising bitopertin, wherein the subject's PPIX levels decrease while the patient's heme levels are substantially maintained. In some embodiments, the subject's heme levels are maintained in comparison to reduced levels of heme observed in a healthy subject administered bitopertin. In some embodiments, the subject's hemoglobin levels are maintained in comparison to reduced levels of hemoglobin observed in a healthy subject administered bitopertin. In some embodiments, the subject's heme and hemoglobin levels are maintained in comparison to reduced levels of heme and hemoglobin observed in a healthy subject administered bitopertin. In some embodiments, no dose dependent decrease in hemoglobin is observed compared to treatment in a subject without EPP. In certain embodiments, the subject's hemoglobin levels are decreased by less than 20%, less than 15%, less than 10%, less than 9%, less than 8%, less than 7%, less than 6%, less than 5%, less than 4%, less than 3%, less than 2%, less than 1%. In certain embodiments, the subject's hemoglobin levels are decreased by less than 10%. In certain embodiments, the subject's hemoglobin levels are decreased by less than 5%. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some embodiments, the subject's PPIX levels decrease by at least 50% (e.g., 50%, 55%, 60%, 65%, 70%, 75%, 80%, 85%, 90%, 95%, or at least 100%) and the subject's hemoglobin levels decrease no more than 10% (e.g., 10%, 15%, 20%, 25%, and 30%). In some embodiments, the subject's PPIX levels decrease by at least 85% and the subject's hemoglobin levels decrease no more than 15%. In some embodiments, the subject's PPIX levels decrease by at least 80% and the subject's hemoglobin levels decrease no more than 15%. In some embodiments, the subject's PPIX levels decrease by at least 75% and the patient's hemoglobin levels decrease no more than 15%. In some embodiments, the subject's PPIX levels decrease by at least 70% and the subject's hemoglobin levels decrease no more than 15%. In some embodiments, the subject's PPIX levels decrease by at least 65% and the subject's hemoglobin levels decrease no more than 15%. In some embodiments, the subject's PPIX levels decrease by at least 60% and the subject's hemoglobin levels decrease no more than 15%. In some embodiments, the subject's PPIX levels decrease by at least 55% and the subject's hemoglobin levels decrease no more than 15%. In some embodiments, the subject's PPIX levels decrease by at least 50% and the subject's hemoglobin levels decrease no more than 15%. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some embodiments, the subject's PPIX levels decrease by at least 50% (e.g., 50%, 55%, 60%, 65%, 70%, 75%, 80%, 85%, 90%, 95%, or at least 100%) and the subject's heme levels decrease no more than 10% (e.g., 10%, 15%, 20%, 25%, and 30%). In some embodiments, the subject's PPIX levels decrease by at least 85% and the subject's heme levels decrease no more than 15%. In some embodiments, the subject's PPIX levels decrease by at least 80% and the subject's heme levels decrease no more than 15%. In some embodiments, the subject's PPIX levels decrease by at least 75% and the patient's heme levels decrease no more than 15%. In some embodiments, the subject's PPIX levels decrease by at least 70% and the subject's heme levels decrease no more than 15%. In some embodiments, the subject's PPIX levels decrease by at least 65% and the subject's heme levels decrease no more than 15%. In some embodiments, the subject's PPIX levels decrease by at least 60% and the subject's heme levels decrease no more than 15%. In some embodiments, the subject's PPIX levels decrease by at least 55% and the subject's heme levels decrease no more than 15%. In some embodiments, the subject's PPIX levels decrease by at least 50% and the subject's heme levels decrease no more than 15%.

In certain aspects, the disclosure relates to methods of treating EPP or XLPP in a subject, the method comprising administering to the subject a pharmaceutical composition comprising bitopertin, wherein the dosage of the pharmaceutical composition does not cause a substantial reduction in heme levels. In some embodiments, the subject's PPIX levels decrease by at least 50% (e.g., 50%, 55%, 60%, 65%, 70%, 75%, 80%, 85%, 90%, 95%, or at least 100%). In some embodiments, the subject's PPIX levels decrease by at least 55%. In some embodiments, the subject's PPIX levels decrease by at least 60%. In some embodiments, the subject's PPIX levels decrease by at least 65%. In some embodiments, the subject's PPIX levels decrease by at least 70%. In some embodiments, the subject's PPIX levels decrease by at least 75%. In some embodiments, the subject's PPIX levels decrease by at least 80%. In some embodiments, the subject's PPIX levels decrease by at least 85%. In some embodiments, the subject's PPIX levels decrease by at least 90%. In some embodiments, the subject's PPIX levels decrease by at least 95%. In some embodiments, the subject's PPIX levels decrease by at least 100%.

In some embodiments, the subject's heme levels decrease no more than 10% (e.g., 10%, 15%, 20%, 25%, and 30%). In some embodiments, the subject's heme levels decrease no more than 15%. In some embodiments, the subject's heme levels decrease no more than 20%. In some embodiments, the subject's heme levels decrease no more than 25%. In some embodiments, the subject's heme levels decrease no more than 30%.

In some embodiments, the subject's PPIX levels decrease by at least 50% (e.g., 50%, 55%, 60%, 65%, 70%, 75%, 80%, 85%, 90%, 95%, or at least 100%) and the subject's heme levels decrease no more than 10%. In some embodiments, the subject's PPIX levels decrease while the subject's heme levels are substantially maintained.

In some embodiments, the accumulation of one or more of the following heme intermediates is inhibited, wherein the one or more heme intermediates is selected from the group consisting of PPIX, ZPPIX, and/or 5-ALA. In some embodiments, the disclosure relates to methods of inhibiting the accumulation of PPIX, the method comprising administering to the subject a pharmaceutical composition comprising bitopertin. In some embodiments, the disclosure relates to methods of inhibiting the accumulation of ZPPIX, the method comprising administering to the subject a pharmaceutical composition comprising bitopertin. In some embodiments, the disclosure relates to methods of inhibiting the accumulation of 5-ALA, the method comprising administering to the subject a pharmaceutical composition comprising bitopertin. In some embodiments, the accumulation of the one or more heme intermediates (e.g., PPIX, ZPPIX, and/or 5-ALA) is inhibited in a dose dependent manner.

Protoporphyrin accumulation in EPP and XLPP can cause liver damage when the hepatic load exceeds the canalicular excretion capacity. The accumulation of PPIX in hepatocytes and bile canaliculi may result in cell damage, cholestasis, cytolysis and further retention of protoporphyrin. Excess protoporphyrin can exert cholestatic effects leading to changes in the hepatobiliary system which can range from mild inflammation to fibrosis and cirrhosis (e.g., cholelithiasis, mild liver disease, deteriorating liver disease, and terminal phase liver disease). Between 3-5% of patients with EPP or XLPP develop protoporphyric hepatopathy, a severe liver disease that can progress rapidly and require liver transplantation. Approximately 2% of patients will develop severe liver disease. There is currently no intervention that is effective in restoring normal liver function once hepatic failure ensues (Anstey, V. and Hift, RJ., Gut, 2007; 56:1009-1018).

In certain aspects, the disclosure provides methods of treating liver damage in a subject. In some embodiments, the liver damage is induced by protoporphyrin IX (PPIX). In some embodiments, the subject has erythropoietic protoporphyria (EPP). In some embodiments, the subject has X-linked protoporphyria (XLPP). In some embodiments, the liver damage is associated with EPP or XLPP. In some embodiments, the liver damage is associated with EPP. In some embodiments, the liver damage is associated with XLPP. In some further embodiments, the liver damage is protoporphyrin IX (PPIX) induced liver damage. PPIX can induce liver damage when accumulation of PPIX in hepatocytes exceeds canalicular excretion capacity. In some embodiments, the disclosure provides methods of treating protoporphyrin IX (PPIX) induced liver damage in a subject. In some embodiments, the subject has erythropoietic protoporphyria (EPP). In some embodiments, the subject has X-linked protoporphyria (XLPP). In some embodiments, the liver damage is associated with EPP or XLPP. In some embodiments, the liver damage is associated with EPP. In some embodiments, the liver damage is associated with XLPP. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

As used herein, the term “hepatopathy” should be given its ordinary meaning, e.g., an abnormal or diseased state of the liver. Hepatopathy, as used herein, also refers to “protoporphyric hepatopathy”, a cholestatic form of liver disease. Protoporphyric hepatopathy is a complication of EPP likely to occur in subjects with high levels of protoporphyrin (i.e., excess protoporphyrin). In certain aspects, the disclosure provides methods of treating hepatopathy. In some embodiments, the hepatopathy is associated with EPP or XLPP in a subject. In some embodiments, the hepatopathy is associated with protoporphyrin accumulation in a subject. In some embodiments, the hepatopathy is induced by protoporphyrin accumulation in a subject. In some embodiments, the hepatopathy is associated with protoporphyrin IX (PPIX) accumulation in a subject. In some embodiments, the hepatopathy is induced by PPIX accumulation in a subject. In some embodiments, administration of bitopertin according to the methods provided herein ameliorates hepatic toxicity. In some embodiments, administration of bitopertin according to the methods provided herein reduces the incidence of hepatobiliary disease. In some embodiments, administration of bitopertin according to the methods provided herein reduces the progression of hepatobiliary disease. Hepatobiliary disease, as used herein, should be given its plain and ordinary meaning. Hepatobiliary disease can include, but is not limited to, disorders of the liver, pancreas, stomach, gallbladder, and/or biliary system.

In certain aspects, the disclosure relates to methods of preventing, treating, or reducing the progression rate and/or severity of liver disease associated with EPP or XLPP in a subject, the method comprising administering to the subject a pharmaceutical composition comprising bitopertin. In some embodiments, the liver disease associated with EPP or XLPP is cholelithiasis. In some embodiments, the liver disease associated with EPP or XLPP is mild liver disease. In some embodiments, the liver disease associated with EPP or XLPP is deteriorating liver disease. In some embodiments, the liver disease associated with EPP or XLPP is terminal phase liver disease. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some embodiments of the methods provided herein, the method comprises administering a GlyT1 inhibitor to the subject. In some embodiments, the method comprises administering a pharmaceutical composition comprising a GlyT1 inhibitor to the subject. In some embodiments, the method comprises administering bitopertin to the subject. In some embodiments, the method comprises administering a pharmaceutical composition comprising bitopertin to the subject. In some embodiments, the method comprises administering 60 mg of bitopertin to the subject. In some embodiments, the method comprises administering a pharmaceutical composition comprising 60 mg of bitopertin to the subject. In some embodiments, the method comprises administering 60 mg of bitopertin to the subject for a treatment period of at least four weeks. In some embodiments, the method comprises administering a pharmaceutical composition comprising 60 mg of bitopertin to the subject for a treatment period of at least four weeks. In some embodiments of the methods provided herein, the treatment period is at least 29 days, at least 71 days, at least 121 days, at least 177 days, at least 233 days, or at least, 289 days. In some embodiments, the treatment period is at least 1 week, at least 4 weeks, at least 8 weeks, at least 16 weeks, at least 17 weeks, at least 20 weeks, at least 30 weeks, at least 40 weeks, at least 50 weeks, or at least 52 weeks. In some embodiments, the treatment period is at least 1 year. In some embodiments, the treatment period is at least 2 years. In some embodiments of the methods provided herein, administering 60 mg of bitopertin to a subject in need thereof for any treatment period provided herein maintains liver function in a subject. In some embodiments, administering 60 mg of bitopertin to a subject in need thereof for a treatment period of at least 17 weeks maintains liver function in a subject. In some preferred embodiments, the disclosure provides methods for the treatment of EPP or XLPP comprising administering 60 mg of bitopertin to a subject in need thereof for at least 17 weeks, wherein the subject maintains liver function. In some embodiments of the methods provided herein, the GlyT1 inhibitor is administered orally. In some embodiments of the methods provided herein, the bitopertin is administered orally. In some embodiments, the pharmaceutical composition is formulated for oral administration. In some embodiments of the methods provided herein, administration alleviates one or more symptoms of liver disease. In some embodiments, the one or more symptoms of liver disease is selected from the group consisting of ascites, encephalopathy, hepatocellular carcinoma, jaundice, and peripheral edema.

In certain aspects, the disclosure provides methods of decreasing porphyrin concentrations in a subject. In some embodiments, the disclosure provides methods of decreasing liver porphyrin concentrations in a subject. In some embodiments, the disclosure provides methods of decreasing liver PPIX concentrations in a subject. In some embodiments, the disclosure provides methods of decreasing bile concentrations of porphyrins in a subject. In some embodiments, the disclosure provides methods of decreasing bile concentrations of PPIX in a subject. In some embodiments, the subject has increased concentrations of porphyrins in the liver relative to baseline. In some embodiments, the subject has increased concentrations of porphyrins in bile relative to baseline. In some embodiments, the porphyrins comprise protoporphyrin IX (PPIX). In some embodiments, the method reduces the concentration of porphyrins by at least 5%, at least 10%, at least 15%, at least 20%, at least 30%, at least 40%, or at least 50%. In some embodiments, the method reduces the concentration of PPIX by at least 5%, at least 10%, at least 15%, at least 20%, at least 30%, at least 40%, or at least 50%. In some embodiments, the method reduces the concentration of PPIX by at least 5%. In some embodiments, the method reduces the concentration of PPIX by at least 10%. In some embodiments, the method reduces the concentration of PPIX by at least 15%. In some embodiments, the method reduces the concentration of PPIX by at least 20%. In some embodiments, the method reduces the concentration of PPIX by at least 30%. In some embodiments, the method reduces the concentration of PPIX by at least 40%. In some embodiments, the method reduces the concentration of PPIX by at least 50%. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

Liver function in patients with EPP and XLPP can be assessed using various known clinical assays. In some embodiments, liver function tests can be used to determine the level of various biochemical parameters (e.g., raised aspartate transaminase levels, alkaline phosphatase, or γ-glutamyl transferase levels). In some embodiments, histopathology of liver biopsies may be used to assess one or more parameters (e.g., protoporphyrin deposition, fibrosis, infiltrates, portal fibrosis, and periportal fibrosis) in the subject. In some embodiments, ultrastructural studies of biopsy specimen can be used to determine if crystal containing vacuoles are present in the subject. With deterioration of liver function, urinary coproporphyrin excretion increases. In some embodiments, coproporphyrin excretion in the urine may be analyzed to assess liver function in the subject. In some embodiments, ultrasound or magnetic resonance elastography may be used to measure liver stiffness in the subject.

Subjects with EPP or XLP can develop hepatic fibrosis due to the excess PPIX presented to the liver for secretion into the bile. Hepatic fibrosis occurs on a spectrum. The level of fibrosis damage can be categorized based upon a hepatic fibrosis score of F0 to F4. A hepatic fibrosis score of F0 indicates no fibrosis. A hepatic fibrosis score of F1 indicates portal fibrosis without septa. A hepatic fibrosis score of F2 indicates portal fibrosis with septa. A hepatic fibrosis score of F3 indicates septal fibrosis without cirrhosis. A hepatic fibrosis score of F4 indicates cirrhosis. The hepatic fibrosis score of F0 to F4 also corresponds to the degree of scarring of the liver. A hepatic fibrosis score of F0 is associated with no scarring of the liver. A hepatic fibrosis score of F1 is associated with mild scarring of the liver. A hepatic fibrosis score of F2 is associated with moderate scarring of the liver. A hepatic fibrosis of F3 is associated with severe scarring of the liver. A hepatic fibrosis score of F4 is associated with cirrhosis of the liver. Hepatic fibrosis may also be scored based upon grades ranging from grade 1 (minimal) to grade 4 (severe). Hepatic fibrosis is traditionally detected using liver biopsy.

Alternative methods have become available to characterize the presence and severity of hepatic fibrosis. The Enhanced Liver Fibrosis (ELF™) test is performed on serum. It consists of 3 fully automated, 2-site sandwich immunoassays using direct chemiluminometric technology. The component assays use 2 monoclonal mouse antibodies: The first antibody is an acridinium ester-labeled antibody; the second is a biotin-labeled antibody. The solid phase contains streptavidin-coated paramagnetic particles. The ELF™ score is derived from an algorithm that combines results for type III procollagen peptide (PIIINP), tissue inhibitor of metalloproteinase-1 (TIMP-1), and hyaluronic acid (HA). Risk cut-offs are established to assess the likelihood of progression to cirrhosis and liver-related clinical events within 3.9 years following baseline ELF score. An ELF score of less than 9.8 indicates a lower risk of progression to cirrhosis. An ELF score of 9.80 to 11.29 indicates a moderate or mid-risk of progression to cirrhosis. An ELF score of greater than 11.29 indicates a higher risk of progression to cirrhosis.

In certain aspects, the disclosure provides methods of treating hepatic fibrosis in a subject. In some embodiments, the hepatic fibrosis is associated with EPP or XLPP in the subject. In some embodiments, the hepatic fibrosis is associated with protoporphyrin accumulation in a subject. In some embodiments, the hepatic fibrosis is induced by protoporphyrin accumulation in a subject. In some embodiments, the hepatic fibrosis is associated with protoporphyrin IX (PPIX) accumulation in a subject. In some embodiments, the hepatic fibrosis is induced by PPIX accumulation in a subject. In some embodiments, the subject does not experience statistically significant abuse potential. In some embodiments, the method does not produce statistically significant abuse potential. In some embodiments, the method does not induce statistically significant abuse potential. In some embodiments, the method does not result in statistically significant abuse potential.

In some embodiments of the methods provided herein, the subject has or is at risk of developing hepatic fibrosis. In some embodiments, the subject is at risk of progression to cirrhosis. In some embodiments, the subject has a low-risk, mid-risk, or high-risk of progression to cirrhosis. In some embodiments of the methods provided herein, the method decreases the risk of progression to cirrhosis. In some embodiments, administration of a GlyT1 inhibitor to the subject decreases the risk of progression to cirrhosis. In some embodiments, administration of a bitopertin to the subject decreases the risk of progression to cirrhosis. In some embodiments, the risk of progression to cirrhosis is determined with an Enhanced Liver Fibrosis (ELF) test. In some embodiments, the subject has an ELF score of less than 9.80. In some embodiments, the subject has an ELF score between 9.80 and 11.29. In some embodiments, the subject has an ELF score greater than 11.29. In some embodiments, the method decreases the subject's ELF score to less than 9.80. In some embodiments, the method decreases the subject's ELF score to less than 11.29. In some embodiments, the method prevents or delays advancement from low-risk to mid-risk of progression to cirrhosis. In some embodiments, the method prevents or delays advancement from mid-risk to high-risk of progression to cirrhosis.

The severity of hepatic fibrosis may also be determined using elastography-based imaging. Elastography utilizes mechanical forces in the form of compression or shear waves to detect changes in soft tissue elasticity. Elastography provides qualitative and quantitative information on tissue stiffness that can be used for further diagnosis. ultrasound elastography or magnetic resonance elastography. Ultrasound elastography pairs ultrasonography (ultrasound) with elastography to image tissues. Ultrasound elastography methods include transient elastography and FibroScan®. Magnetic resonance elastography couples elastography with magnetic resonance imaging (MRI) to image tissues.

In some embodiments of the methods provided herein, the severity of hepatic fibrosis in a subject is determined via elastography. In some embodiments, the severity of hepatic fibrosis is determined via ultrasound elastography. In some embodiments, the severity of hepatic fibrosis is determined via magnetic resonance elastography. In some embodiments, the severity of hepatic fibrosis is determined via FibroScan. In some embodiments, the subject has a hepatic fibrosis score of F0, F1, F2, F3, or F4. In some embodiments, the subject has a hepatic fibrosis score of F0 and has no scarring of the liver. In some embodiments, the subject has a hepatic fibrosis score of F1 and has mild scarring of the liver. In some embodiments, the subject has a hepatic fibrosis score of F2 and has moderate scarring of the liver. In some embodiments, the subject has a hepatic fibrosis score of F3 and has severe scarring of the liver. In some embodiments, the subject has a hepatic fibrosis score of F4 and has cirrhosis of the liver. In some embodiments, administration of a GlyT1 inhibitor, e.g., bitopertin, according to the methods provided herein decreases the subject's hepatic fibrosis score. In some embodiments, administration of bitopertin decreases a subject's hepatic fibrosis score. from F4 to F3. In some embodiments, administration of bitopertin decreases the subject's hepatic fibrosis score from F3 to F2. administration of bitopertin decreases the subject's hepatic fibrosis score from F2 to F1. In some embodiments, administration of bitopertin decreases the subject's hepatic fibrosis score from F1 to F0. In some embodiments, administration of bitopertin prevents or delays progression of the subject's hepatic fibrosis score from F1 to F2, F3, or F4. In some embodiments, administration of bitopertin prevents or delays progression of the subject's hepatic fibrosis score from F2 to F3 or F4. In some embodiments, administration of bitopertin prevents or delays progression of the subject's hepatic fibrosis score from F3 to F4.

Liver stiffness can be detected using elastography as provided herein. Liver stiffness is quantified in units of kilopascals (kPa). Liver stiffness values below 6 kPa are considered as normal and exclude liver disease. Values greater than 8 and 12.5 kPa are indicative of advanced fibrosis and cirrhosis, respectively (i.e., hepatic score of F3 and F4). In some further embodiments of the methods provided herein, the subject has a liver stiffness greater than 2 kPa. In some embodiments, the subject has a liver stiffness greater than 7 kPa. In some embodiments, the subject has a liver stiffness greater than 9 kPa. In some embodiments, the subject has a liver stiffness greater than 11 kPa. In some embodiments, the subject has a liver stiffness greater than 14 kPa. In some embodiments, the subject has a liver stiffness greater than 17 kPa. In some embodiments, the methods provided herein decrease the subject's liver stiffness to less than 17 kPa. In some embodiments, the methods provided herein decrease the subject's liver stiffness to less than 14 kPa. In some embodiments, the methods provided herein decrease the subject's liver stiffness to less than 11 kPa. In some embodiments, the methods provided herein decrease the subject's liver stiffness to less than 9 kPa. In some embodiments, the methods provided herein decrease the subject's liver stiffness to less than 7 kPa. In some embodiments, the methods provided herein delay or prevent increases in the subject's liver stiffness.

A subject with liver disease, hepatopathy, hepatic fibrosis, and/or liver damage may have elevated levels of serum hepatobiliary biomarkers relative to baseline. In certain aspects, the disclosure provides methods for reducing hepatobiliary biomarkers of liver damage in a subject. In some embodiments, the subject has elevated levels of one or more hepatobiliary biomarkers compared to baseline. In some embodiments, the one or more hepatobiliary biomarkers is selected from the group consisting of aspartate aminotransferase (AST), alanine aminotransferase (ALT), alkaline phosphatase (AP), Gamma-glutamyl transpeptidase (GGT), bilirubin, alpha-2 macroglobulin (A2M), apolipoprotein A1 (ApoA1), haptoglobin (Hp), albumin, 5′-nucleotidase (5′-NT), and primary serum bile acids. In some embodiments, the subject, when compared to baseline, has elevated levels of one or more hepatobiliary biomarkers selected from the group consisting of aspartate aminotransferase (AST), alanine aminotransferase (ALT), alkaline phosphatase (AP), Gamma-glutamyl transpeptidase (GGT), bilirubin, alpha-2 macroglobulin (A2M), apolipoprotein A1 (ApoA1), haptoglobin (Hp), albumin, 5′-nucleotidase (5′-NT), and primary serum bile acids.

In some embodiments, the methods provided herein reduce serum concentrations of bile acids. Elevated levels of serum bile acids have been measured in patients with various liver diseases. Bile acids are a product of cholesterol catabolismin the liver. Primary bile acids are synthesized by the liver and include cholic acid (CA) and chenodeoxycholic acid (CDCA). Secondary bile acids are those that are subsequently modified by gut bacteria and include deoxycholic acid and lithocholic acid. In some embodiments, the methods provided herein reduce serum primary bile acid levels. In some embodiments, the method reduces serum primary bile acid levels by at least 5% in the subject. In some embodiments, the method reduces serum primary bile acid levels by at least 10% in the subject. In some embodiments, the method reduces serum primary bile acid levels by at least 15% in the subject.

Serum alanine aminotransferase (ALT; also referred to as alanine transaminase) levels can be measured as a biomarker for liver disease. ALT is an enzyme most abundant in the cytosol of hepatocytes. Upon incidence of liver injury or liver disease, ALT is released from hepatocytes and causes an elevation in serum levels of ALT activity. In some embodiments, the methods provided herein reduce serum ALT levels in a subject with elevated ALT levels relative to baseline. In some embodiments, the method reduces ALT levels in the subject by at least 5%. In some embodiments, the method reduces ALT levels in the subject by at least 10%. In some embodiments, the method reduces ALT levels in the subject by at least 15%.

Serum aspartate aminotransferase (AST) levels can be measured as a biomarker for hepatic injury. AST is an enzyme primarily found in hepatocytes. During hepatic injury, damage to hepatocytes releases AST, thereby causing an increase in serum AST levels. In addition to AST levels, the ratio of AST to ALT levels is quantified to distinguish between extrahepatic versus hepatic injury. AST/ALT ratios of 2:1 are indicative of hepatic injury. In some embodiments, the methods provided herein reduce serum AST levels in a subject with elevated AST levels relative to baseline. In some embodiments, the method reduces AST levels in the subject by at least 5%. In some embodiments, the method reduces AST levels concentrations in the subject by at least 10%. In some embodiments, the method reduces AST levels in the subject by at least 15%.

Alkaline phosphatase (AP) is an enzyme with high concentration in the liver, bile ducts, and bones. Elevated serum AP relative to baseline can be indicative of liver damage. In some embodiments, the methods provided herein reduce serum levels of AP in a subject. In some embodiments, the method reduces AP concentrations in the subject by at least 5%. In some embodiments, the method reduces AP concentrations in the subject by at least 10%. In some embodiments, the method reduces AP concentrations in the subject by at least 15%.

Gamma-glutamyl transferase (GGT) is an enzyme primarily found in bile ducts. GGT has been used as a biomarker for liver disease and/or damage to the bile ducts. Increased serum levels of GGT might indicate liver damage and/or liver disease. In some embodiments, the methods provided herein reduce serum levels of GGT in a subject. In some embodiments, the method reduces GGT concentrations in the subject by at least 5%. In some embodiments, the method reduces GGT concentrations in the subject by at least 10%. In some embodiments, the method reduces GGT concentrations in the subject by at least 15%.

Bilirubin is a bile pigment that is elevated in plasma upon liver damage. Bilirubin is a known biomarker of liver dysfunction, and serum total bilirubin (TBR) concentrations can be measured as an assessment of liver function. In some embodiments, the methods provided herein reduce serum total bilirubin (TBR) concentrations in a subject relative to baseline. In some embodiments, the method reduces serum TBR concentrations in the subject by at least 5%. In some embodiments, the method reduces serum TBR concentrations in the subject by at least 10%. In some embodiments, the method reduces serum TBR concentrations in the subject by at least 15%.

Alpha-2-macroglobulin (A2M) is a protease inhibitor synthesized by liver parenchymal cells. Increased A2M levels can be used as a biomarker of liver fibrosis and inflammatory activity in liver disease. In some embodiments, the methods provided herein reduce serum A2M levels in a subject relative to baseline. In some embodiments, the method reduces A2M concentrations in the subject by at least 5%. In some embodiments, the method reduces A2M concentrations in the subject by at least 10%. In some embodiments, the method reduces A2M concentrations in the subject by at least 15%.

Apolipoprotein A1 (ApoA1) is a protein that forms high-density lipoprotein (HDL) and plays a role in liver cirrhosis. ApoA1 is synthesized in the liver and small intestine, and can be used as a biomarker for liver cirrhosis. Elevated levels of ApoA1 can be associated with cirrhosis. In some embodiments, the methods provided herein reduce serum ApoA1 levels in a subject relative to baseline. In some embodiments, the method reduces ApoA1 concentrations in the subject by at least 5%. In some embodiments, the method reduces ApoA1 concentrations in the subject by at least 10%. In some embodiments, the method reduces ApoA1 concentrations in the subject by at least 15%.

Haptoglobin (Hp) is a glycoprotein that binds free hemoglobin in plasma. Hb is synthesized in the liver and the lungs. Hb can be used as a biomarker for liver disease, where accumulation of Hb and site-specific Hb glycoforms have been found to be associated with cirrhosis and hepatocarcinoma pathologies. The liver facilitates removal of serum haptoglobin-hemoglobin complexes. Low levels Hp levels have been associated with liver disease. In some embodiments, the methods provided herein increase serum Hp levels in a subject relative to baseline. In some embodiments, the method increases serum Hp concentrations in the subject by at least 5%. In some embodiments, the method increases serum Hp concentrations in the subject by at least 10%. In some embodiments, the method increases serum Hp concentrations in the subject by at least 15%.

Serum albumin is synthesized in the liver and is one of the most abundant proteins in human plasma. Low serum albumin concentrations (hypoalbuminemia) can be used as a biomarker for liver cirrhosis, which impairs hepatic serum albumin synthesis. In some embodiments, the methods provided herein increase serum albumin levels in a subject relative to baseline. In some embodiments, the method increases serum albumin levels in the subject by at least 5%. In some embodiments, the method increases serum albumin levels in the subject by at least 10%. In some embodiments, the method increases serum albumin levels in the subject by at least 15%. 5′-nucleotidase (5′-NT) is a protein synthesized in the liver that serves as a biomarker for liver damage. 5′-NT is increased in hepatobiliary disorders. Increased levels of 5′-NT can indicate cholestasis, liver damage, and hepatitis, among other disorders. In some embodiments, the methods provided herein reduce 5′-NT levels in a subject relative to baseline. In some embodiments, the method reduces 5′-NT levels in the subject by at least 5%. In some embodiments, the method reduces 5′-NT levels in the subject by at least 10%. In some embodiments, the method reduces 5′-NT levels in the subject by at least 15%.

FIB-4 is an index utilized to estimate hepatic fibrosis. It calculates a hepatic fibrosis index using a subject's age in years, serum AST [U/L], serum ALT [U/L], and platelet count [x 10⁻⁹/L or 10⁻³/uL]. Higher numbers suggest a higher likelihood of hepatic fibrosis or cirrhosis. The formula for FIB-4 is: Age ([yr]×AST [U/L])/((PLT [10(9)/L])×(ALT [U/L])(1/2)). Subjects with an index of greater than or equal to 3.25 are considered within the high-risk category for hepatic fibrosis. Subjects with an index of less than 1.3 are considered within the low-risk category for hepatic fibrosis. In some embodiments, the methods provided herein reduce a FIB-4 index in a subject relative to baseline. In some embodiments, the subject has a FIB-4 index less than 1.00. In some embodiments, the subject has a FIB-4 index less than 1.30. In some embodiments, the subject has a FIB-4 index greater than 2.65. In some embodiments, the subject has a FIB-4 index greater than 3.25. In some embodiments, the method decreases the subject's FIB-4 index to less than 1.3. In some embodiments, the method decreases the subject's FIB-4 index to less than 2.65. In some embodiments, the method decreases the subject's FIB-4 index to less than 3.25.

Aspartate Aminotransferase to Platelet Count Ratio (APRI) is a serum AST to platelet count ratio index. It is calculated as follows: AST [IU/L]/40 divided by platelet count [10-9/L]×100. In a meta-analysis of 40 studies, investigators concluded that an APRI score greater than 1.0 had a sensitivity of 76% and specificity of 72% for predicting cirrhosis. In addition, they concluded that an APRI score greater than 0.7 had a sensitivity of 77% and specificity of 72% for predicting significant hepatic fibrosis. For detection of cirrhosis, using an APRI cutoff score of 2.0 was more specific (91%) but less sensitive (46%). The lower the APRI score (less than 0.5), the greater the negative predictive value (and ability to rule out cirrhosis) and the higher the value (greater than 1.5) the greater the positive predictive value (and ability to rule in cirrhosis). In some embodiments, the methods provided herein reduce a subject's APRI score relative to baseline. In some embodiments, the subject has an APRI score greater than 0.7. In some embodiments, the subject has an APRI score greater than 1.0. In some embodiments, the subject has an APRI score greater than 1.5. In some embodiments, the subject has an APRI score greater than 2.0. In some embodiments, the method decreases the subject's APRI score to less than 2.0. In some embodiments, the method decreases the subject's APRI score to less than 1.5. In some embodiments, the method decreases the subject's APRI score to less than 1.0.

Measurements of porphyrins in bile, obtained from the duodenum and from liver biopsies, as well as hepatic histopathology, can be obtained from a subject at baseline and following a treatment period with one or more GlyT1 inhibitors (e.g., bitopertin) of the present disclosure. In some embodiments of the methods provided herein, the subject has increased concentrations of porphyrins in the liver relative to baseline. In some embodiments, the subject has increased concentration of porphyrins in bile relative to baseline. In some embodiments, the porphyrins comprise PPIX. In some embodiments, the methods of the present disclosure reduce the concentrations of porphyrins by at least 5%, at least 10%, at least 15%, at least 20%, at least 30%, at least 40%, or at least 50%.

The dosage of bitopertin can be modified for certain patients with hepatic impairment. “Hepatic impairment”, as used herein, is given its usual and ordinary meaning, namely, liver dysfunction. In certain embodiments, the present disclosure provides methods of treating erythropoietic protoporphyria (EPP) or X-linked protoporphyria (XLPP) in a subject with hepatic impairment. In some embodiments, the subject has EPP and hepatic impairment. In some embodiments, the subject has EPP and hepatic dysfunction. In some embodiments, the subject has XLPP and hepatic impairment. In some embodiments, the subject has XLPP and hepatic dysfunction. In some embodiments, the subject is administered a therapeutically effective dosage of 60 mg bitopertin once daily. In some embodiments, the subject is administered a therapeutically effective dosage of 30 mg bitopertin once daily. In some embodiments, the subject is administered a modified amount of or modified therapeutically effective dosage bitopertin once daily. In some embodiments, the subject is administered a modified amount of or modified therapeutically effective dosage of 30 mg bitopertin once daily.

The Child-Pugh scoring system is a clinical assessment of the severity of, e.g., hepatic impairment, hepatic dysfunction, and/or liver disease. The Child-Pugh scoring system evaluates clinical and laboratory criteria including: serum bilirubin, serum albumin, ascites, neurological disorder, and clinical nutrition status; to categorize patients based upon liver function. A classification of Child-Pugh class A (a Child-Pugh score of 5 to 6) indicates normal liver function and/or mild hepatic impairment. A classification of Child-Pugh class B (a Child-Pugh score of 7 to 9) indicates reduced liver function, and/or moderate hepatic impairment. A classification of Child-Pugh class C (a Child-Pugh score of 10 to 15) indicates severe liver dysfunction, and/or severe hepatic impairment.

In some embodiments of the methods and uses provided herein, the subject's hepatic impairment is classified based upon the Child-Pugh scoring system. In some embodiments, the hepatic impairment is mild hepatic impairment. In some embodiments, the subject has a Child-Pugh score of 5 to 6. In some embodiments, the subject is in Child-Pugh class A. In some embodiments, the hepatic impairment is moderate hepatic impairment. In some embodiments, the subject has a Child-Pugh score of 7 to 9. In some embodiments, the subject is in Child-Pugh class B. In some embodiments, the hepatic impairment is severe hepatic impairment. In some embodiments, the subject has a Child-Pugh score of 10 to 15. In some embodiments, the subject is in Child-Pugh class C.

In certain embodiments of the methods and uses as disclosed herein, at least 50%, at least 60%, at least 70%, at least 80%, at least 85%, at least 90%, or at least 95% cell viability is maintained. In certain such embodiments, at least 90% cell viability is maintained.

The present disclosure also provides the following non-limiting embodiments:

In order that the embodiments disclosed herein may be more efficiently understood, examples are provided below. It should be understood that these examples are for illustrative purposes only and are not to be construed as limiting the embodiments in any manner. Throughout these examples, there may be molecular cloning reactions, and other standard recombinant DNA techniques described and these were carried out according to methods described in Maniatis et al., Molecular Cloning-A Laboratory Manual, 2nd ed., Cold Spring Harbor Press (1989), using commercially available reagents, except where otherwise noted.

The following examples are illustrative, but not limiting, of the methods and compositions described herein. Other suitable modifications and adaptations of the variety of conditions and parameters normally encountered in therapy, synthesis, and other embodiments disclosed herein are within the spirit and scope of the embodiments.

### Further Numbered Embodiments of the Invention

The disclosure additionally provides the following numbered embodiments (“Embodiment(s)”) which may also be combined with various other features of the disclosure described herein:
1. A method of treating EPP or XLPP, or related syndrome thereof (e.g., EPP-related syndrome or XLPP-related syndrome) in a subject without statistically significant abuse potential.
2. A method of increasing sunlight tolerance in a subject with EPP or XLPP without statistically significant abuse potential
3. A method of reducing PPIX levels in a subject with EPP or XLPP, without statistically significant abuse potential.
4. A method of increasing sunlight tolerance in a subject with EPP or XLPP comprising administering to the subject 60 mg of bitopertin for a treatment period of at least 17 weeks resulting in a statistically significant increase in sunlight tolerance without statistically significant abuse potential.
5. The method of any one of embodiments 1-4, wherein bitopertin has CNS activity without statistically significant abuse potential.
6. The method of any one of embodiments 1-5, wherein bitopertin penetrates the CNS without statistically significant abuse potential.
7. The method of any one of embodiments 1-6, wherein bitopertin does not demonstrate statistically significant binding to one or more CNS receptor.
8. The method of any one of embodiments 1-7, wherein bitopertin does not demonstrate statistically significant binding to one or more CNS receptor at concentrations up to 10 μM.
9. The method of embodiment 8, wherein the one or more CNS receptor is selected from the group consisting of dopamine receptor, norepinephrine receptor, serotonin receptor, GABA receptor, acetylcholine receptor, opioid receptor, NMDA receptor, and cannabinoid receptor.
10. The method of embodiment 8 or 9, wherein bitopertin exhibits at least 300-fold greater selectivity for GlyT1 than for the one or more CNS receptor.
11. The method of any one of embodiments 1-10, bitopertin does not affect the uptake or release of dopamine, serotonin, or norepinephrine.
12. The method of any one of embodiments 1-11, wherein bitopertin does not bind to or affect the activity of any receptor, enzyme, or channel that may lead to abuse potential.

### EXAMPLES

### Example 1: Synthesis of Bitopertin

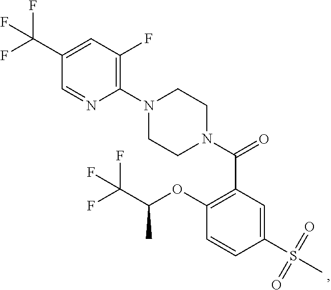 [SMILES: C[C@H](Oc1ccc(S(C)(=O)=O)cc1C(=O)N1CCN(c2ncc(C(F)(F)F)cc2F)CC1)C(F)(F)F]

Bitopertin, can be prepared in accordance with the synthetic protocols provided in U.S. Pat. Nos. 7,319,099, 9,877,963, and 7,812,161, the contents of which are hereby incorporated by reference in their entirety.

### Example 2: Use of Bitopertin to Reduce PPIX and Phototoxicity in Transgenic EPP and XLP Mouse Models

Bitopertin was evaluated as a treatment for EPP was in Fech^{m1Pas} EPP and Alas2^{Q548X} XLP mouse models. The recessive Fech^{m1Pas} allele is an ethylnitrosourea (ENU)-induced missense mutation that retains approximately 5% residual ferrochelatase activity. The Alas2^{Q548X} animals were generated by employing CRISPR-Cas9 editing technology to introduce a known human gain-of-function mutation of Alas2 gene. Overall, the Fech^{m1Pas}/Fech^{m1Pas} homozygous mice and Alas2^{Q548X} mice develop protoporphyria characterized by elevated RBC and liver PPIX levels. Fech^{m1Pas}/Fech^{m1Pas} and Alas2^{Q548X} mice were fed with a diet containing 0 or 100 ppm bitopertin for 8 weeks starting at 6 weeks of age. At 8 weeks, PPIX levels had decreased in Fech^{m1Pas}/Fech^{m1Pas} and Alas2^{Q548X} animals receiving bitopertin with a mean reduction of 43% and 66%, respectively. Importantly, no clinically meaningful changes in Hgb levels were observed in the treated animals. The plasma concentration of bitopertin at termination was generally in the range of exposures observed at doses of 10 and 30 mg of bitopertin in humans.

Treatments that can target the underlying pathophysiology of EPP by reducing PPIX may also prevent or treat EPP-associated hepatopathy. Hepatotoxicity is the most serious and life-threatening complication of EPP, as it progresses rapidly and can necessitate liver transplantation or even lead to death. Clinical presentations of PPIX-induced liver injury range from mild inflammation to fibrosis, cirrhosis, and liver failure (Coffey, 2018; Lyoumi, 2011; Anstey, 2007). An estimated 20 to 30% of patients with EPP have elevations in serum aminotransferases (Balwani, 2019a). Approximately 20% of EPP patients develop cholelithiasis, and 2 to 5% of patients experience severe liver damage and progress to hepatic failure (Di Pierro, 2022). Reductions in PPIX with bitopertin treatment were also shown to ameliorate liver fibrosis in EPP mouse models (Fech^{m1Pas} mice). Bitopertin-treated mice had a statistically significant decrease in liver fibrosis compared to mice fed control diets; the magnitude of these effects was correlated with decreases in blood PPIX levels. Data not shown.

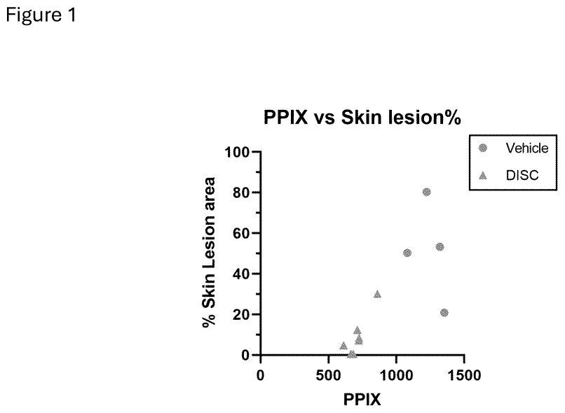

In a separate phototoxicity study, GlyT1 inhibition significantly ameliorated skin lesions in Fech^{m1Pas} EPP mice after ultraviolet (UV)/blue light exposure, and the degree of skin lesions correlated with a 40% reduction in PPIX levels in red blood cells (RBCs). See FIG. 1. FECH^{m1pas}/FECH^{m1pas} EPP mice were dosed with either vehicle (n=4) or the GlyT1 inhibitor, DISC-C (n=7) 15 mg/kg BID for 18 days. On Day 14 of treatment, hair was removed from the backs of the EPP mice, and all mice were exposed to light with wavelength of 395 nM, 588±10% lumens (lm)/m² for 30 minutes. The exposed area of skin that developed lesions was quantitatively assessed on Day 18 (4 days after UV/blue light exposure).

Overall, the animal model data indicate that bitopertin can reduce PPIX accumulation without significant impact on erythropoiesis and can address hepatobiliary complications and severe cutaneous manifestations of EPP.

### Example 3: Phase 2, Randomized, Double-Blind, Placebo-Controlled Study to Evaluate the Efficacy, Safety, and Tolerability of Bitopertin in the Treatment of Erythropoietic Protoporphyria (EPP)

Participants ≥18 years of age with a confirmed diagnosis of EPP or XLP by genetic testing or porphyrin analysis were enrolled in the study. Exclusion criteria included aspartate aminotransferase/alanine aminotransferase values ≥2× the upper limit of normal, bilirubin >upper limit of normal, hemoglobin <10 g/dL, or concurrent treatment with afamelanotide or dersimelagon. 75 adult participants with EPP (1:1:1) received oral, QD administration of 20 mg bitopertin, 60 mg bitopertin, or placebo for 17 weeks. Randomization was stratified by baseline light tolerance (time to prodrome <30 minutes or ≥30 minutes), as assessed over a 2-week screening period. The primary efficacy endpoint was the percent change in whole-blood metal-free PPIX levels, and the key secondary efficacy endpoint was total hours of sunlight exposure to skin on days with no pain from 10:00 to 18:00 hours. Additional assessments included patient-reported outcomes (PROs) of light tolerance and PRO measures of symptoms and symptom impacts, changes in liver injury biomarkers and fibrosis, and safety and tolerability. Table 2 provides the efficacy endpoints for the phase 2 trial to evaluate the efficacy, safety, and tolerability of bitopertin in the treatment of Erythropoietic Protoporphyria (EPP).

TABLE 2

| | |
|---|---|
| Primary Endpoint | Percent change from baseline in whole-blood-metal-free protoporphyrin |
| | IX (PPIX) at Day 121 (approximately 17 weeks) |
| Key Secondary | Total hours of sunlight exposure to skin on days with no pain from 10:00 |
| Endpoint | to 18:00 (10:00 AM to 6:00 PM) from Randomization to Day 121 |
| Secondary | Change in 2-week average daily sunlight exposure time (minutes) to |
| Endpoints | first prodromal symptom (e.g., burning, tingling, itching, or stinging) |
| | associated with sunlight exposure between 1 hour post-sunrise and 1 |
| | hour pre-sunset through Day 121 |
| | Pain intensity of phototoxic reactions according to a Likert scale (0-10) |
| | Safety and tolerability of bitopertin, as assessed by the incidence of |
| | treatment-emergent adverse events (TEAEs) |
| | Erythrocyte metal-free PPIX concentrations at Day 121 |
| | Plasma and whole blood total PPIX concentrations at Day 121 |
| Exploratory | Number of phototoxic reactions with a pain component (recorded in a |
| Endpoints | diary) through Day 121 |
| | Time to recovery of phototoxic reactions with a pain component |
| | Change in time to prodromal symptom in sunlight on a single day, |
| | assessed once weekly, from Week 5 through Day 121 |
| | Changes from baseline in daily light dosimetry measurements |
| | Change from baseline in patient-reported outcome (PRO) scores from: |
| | Patient-Reported Outcomes Measurement Information System |
| | (PROMIS)-57 V2.1 PROMIS-Short Form v2.0 - Social Isolation 8a, |
| | PROMIS v2.0, and PROMIS Neuropathic Pain Quality v2.0 5a |
| | Change in Patient Global Impression of Change (PGIC) scores |
| | Change in Patient Global Impression of Severity (PGIS) scores |

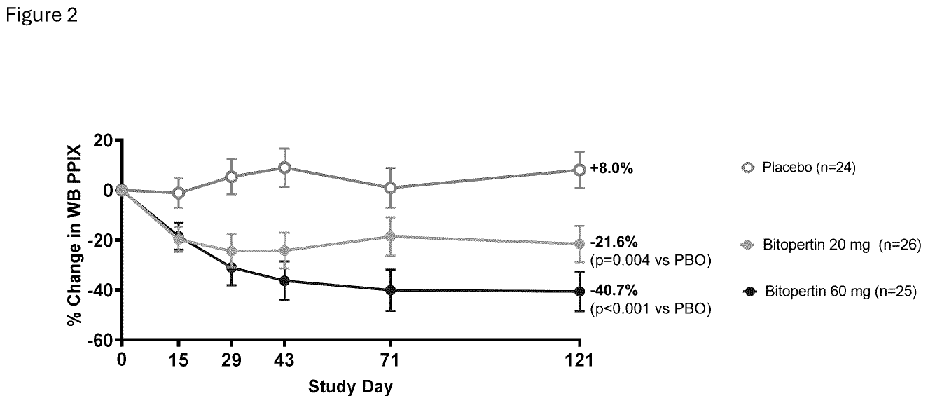

Treatment with bitopertin resulted in significant and sustained reductions in PPIX levels compared to PPIX levels for treatment with placebo at both the 20 mg and 60 mg dose levels (least-squares [LS] mean difference relative to placebo: −29.7% [p=0.004] and −48.7% [p<0.001], respectively). The reductions in PPIX with bitopertin were observed across several prespecified subgroups, including age, sex, and disease severity. Treatment with bitopertin significantly reduced the rate of phototoxic reactions compared to the rate observed in treatment with placebo; the incidence rate ratios relative to those of placebo were 0.397 (p=0.11) in the 20 mg bitopertin group and 0.247 (p=0.01) in the 60 mg bitopertin group, corresponding to 60% and 75% reductions in the phototoxic reaction rate compared to the rate in the placebo group. PPIX reductions were associated with improvements in multiple sunlight tolerance measures, including cumulative total pain-free time in sunlight, time to first prodromal symptom, occurrence of phototoxic reactions, and improvements in how participants reported feeling in the Patient Global Impression of Change. A post hoc longitudinal mixed model for repeated-measures analysis showed nominally statistically significant, time-dependent improvements in pain-free sunlight exposure with bitopertin compared to the improvements with placebo in both the 20 mg and 60 mg dose groups (LS mean [±SE] difference relative to placebo: 7.1±3.2 hours [p=0.026] and 8.0±3.2 hours, [p=0.013], respectively), and participants randomized to bitopertin had a 2-fold improvement in average daily sunlight exposure at the end of study relative to baseline. See FIG. 2. For the primary endpoint, treatment with bitopertin resulted in significant and sustained reductions in PPIX levels compared to placebo at both the 20-mg and 60-mg dose levels. The reductions were dose-dependent with more pronounced decreases observed with 60 mg (−40.7% [p<0.001 vs placebo]) compared to 20 mg (−21.6% [p=0.003 versus placebo]).

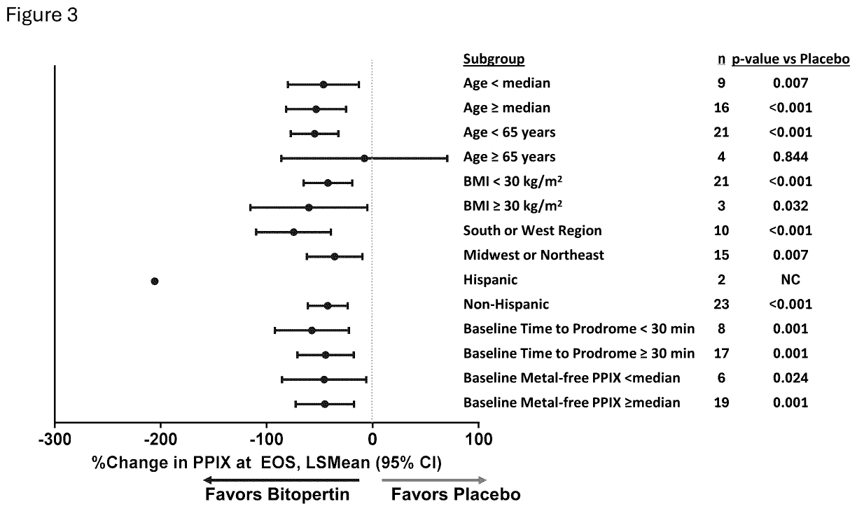

The reductions in PPIX with bitopertin were observed across many prespecified subgroups, including sex, age, and measures of disease severity, as assessed by baseline PPIX levels (<median or ≥median for the randomized intent-to-treat [ITT] population) and baseline light tolerance (time to prodrome <30 minutes or ≥30 minutes). See FIG. 3.

The key secondary endpoint utilized in this study is defined as follows: total hours of sunlight exposure on days with no pain due to a full reaction from 10:00 to 18:00 (10:00 am to 6:00 μm) summed over the entire treatment period from randomization to Day 121. Participants were asked to complete a sunlight exposure diary through an electronic portal daily. The amount of time recorded in diaries from relevant study days were summed to obtain daily daylight tolerance. An analysis of variance (ANOVA) model was used for the ITT population to compare least-squares (LS) means and pairwise differences between LS means of bitopertin and placebo groups. Diary completion during the 17-week double-blind treatment period was high. Assuming 121 expected diary entries, the completion percentages were 85%, 80%, and 88% in the 20-mg bitopertin, 60-mg bitopertin, and placebo groups, respectively. The statistical analysis plan prespecified handling rules for missing data. If the diary was not completed on a given day or if reactions to sunlight exposure (phototoxic reaction, or no symptoms) were missing, daily daylight tolerance was imputed under a missing at random (MAR) assumption for the ITT population. The imputation process imputed data for the missing diary days based on the observed daylight tolerance on days with no pain from a phototoxic reaction.

PPIX reductions were associated with increased sunlight tolerance, as assessed by 3 different measures: 1) time in light without pain (hours); 2) change from baseline in time to prodrome (minutes); and 3) the occurrence of phototoxic reaction. See Table 3 which shows clinical outcome measures by tertiles of PPIX change. PGIC=Patient Global Impression of Change; PPIX=protoporphyrin IX. Only 57 patients completed sun exposure challenges at baseline and during the study period to calculate change from baseline in time to prodrome (n=20 in tertile 1, n=18 in tertile 2, n=19 in tertile 3).

TABLE 3

| | PPIX Increased | | PPIX Decreased |
|---|---|---|---|
| | 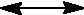 | | |
| | Tertile 3 | Tertile 2 | Tertile 1 |
| | (−7% to 190%) | (−38% to −7%) | (−88% to −38%) |
| | n = 25 | n = 24 | n = 24 |
| Treatment Assignment | Placebo, n = 17 | Placebo, n = 5 | Placebo, n = 2 |
| | Bitopertin 20 mg, n = 7 | Bitopertin 20 mg, | Bitopertin 20 mg, n = 6 |
| | Bitopertin 60 mg, n = 1 | n = 13 | Bitopertin 60 mg, n = 16 |
| | | Bitopertin 60 mg, n = 6 | |

Light Tolerance Measure (Mean ± SE)

| | | | |
|---|---|---|---|
| Cumulative total time | 117.5 ± 16.6 | 124.5 ± 13.9 | 161.1 ± 19.1 |
| in light without pain | | | |
| (hr) | | | |
| Average daily time in | 1.16 ± 0.17 | 1.20 ± 0.15 | 1.61 ± 0.27 |
| light without pain (hr) | | | |
| Change from baseline | 64.1 ± 8.4 | 109.4 ± 28.5 | 117.4 ± 33.2 |
| in | | | |
| time to prodrome (min)^{a} | | | |
| Occurrence of | 8 (32%) | 9 (38%) | 2 (8%) |
| phototoxic | | | |
| reaction (n, %) | | | |
| Occurrence of | 4 (16%) | 5 (21%) | 1 (4%) |
| phototoxic | | | |
| reaction in last 60 days | | | |
| (n, %) | | | |

PGIC Response (n, %)

| | | | |
|---|---|---|---|
| Much better | 12 (48%) | 18 (75%) | 21 (91%) |
| A little better | 5 (20%) | 1 (4%) | 0 |
| No change/A little | 8 (32%) | 5 (21%) | 2 (9%) |
| worse | | | |
| Did not respond | 0 | 0 | 1 (4%) |

Participants in the tertile with the greatest PPIX reductions also experienced fewer phototoxic reactions, while PPIX increases were associated with increased occurrence of phototoxic reactions. Among participants randomized to bitopertin, greater PPIX reductions were observed at the end of study in participants who reported no phototoxic reaction (−36.5%, n=41) compared to those who reported phototoxic reactions (−4.0%, n=8). Tertile analyses also showed that PPIX reductions were associated with improved PGIC responses. In the tertile of participants who had the greatest PPIX reductions, a larger proportion of participants reported feeling “Much better.” Collectively, PPIX reductions were associated with improvements across multiple assessments evaluating the full spectrum of sunlight tolerance, including cumulative or average daily sunlight exposure, time to first prodromal symptom (early warning symptoms of a phototoxic reaction), and occurrence of phototoxic reactions, as well as improvements in how participants reported feeling.

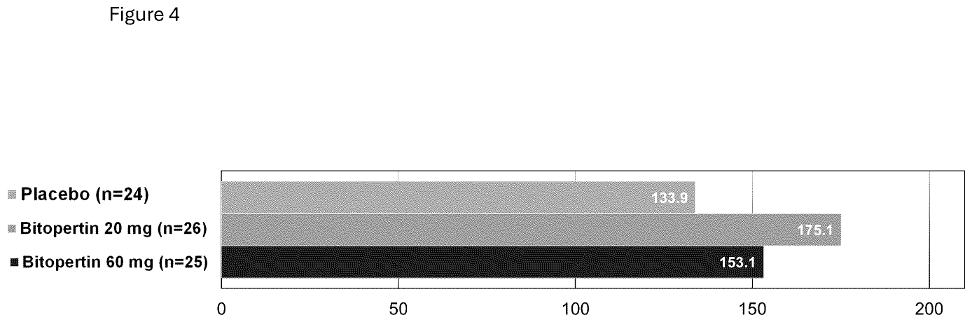

For the key secondary endpoint, the mean cumulative total time in light observed over the 4-month treatment period on days without pain was numerically higher with bitopertin than placebo: 175.1 hours in the 20 mg dose group and 153.1 hours in the 60 mg dose group, compared to 133.9 hours in participants randomized to placebo. See FIG. 4. The corresponding mean differences relative to placebo in the 20 mg and 60 mg bitopertin groups were 41.2 hours (p=0.16) and 19.2 hours (p=0.52), respectively. In addition, a prespecified set of sensitivity analyses were performed to evaluate total time in light with no pain using data collected from the daily sunlight exposure diary using alternate windows for daily sun exposure:
Daily daylight tolerance on days with no pain from 06:00 to 22:00 hours (all possible hours)
Daily daylight tolerance on days with no pain from 10:00 to 15:00 hours (peak sunlight hours)

Total hours of sunlight exposure on days with no pain using the alternate sun exposure windows (06:00 to 22:00 or 10:00 to 15:00) were summed over the entire treatment period from randomization to Day 121 and applied the same methodology used to analyze the key secondary endpoint. See Table 4 which shows total hours of sunlight exposure on days with no pain using the alternate sun exposure windows (06:00 to 22:00 or 10:00 to 15:00), summed over the entire treatment period from randomization to Day 121 for each of placebo, bitopertin 20 mg and bitopertin 60 mg.

TABLE 4

| Sunlight Exposure | Bitopertin | Bitopertin | Placebo |
|---|---|---|---|
| Window | 20 mg (n = 26) | 60 mg (n = 25) | (n = 24) |
| 10:00 to 18:00 | | | |
| LS means (95% | 175.1 (134.7, 215.5) | 153.1 (111.7, 194.6) | 133.9 (92.2, 175.6) |
| CI) | | | |
| Difference of LS | 41.2 (−16.1, 98.5) | 19.2 (−38.7, 77.1) | — |
| means (95% CI) | | | |
| p-value vs placebo | 0.16 | 0.52 | — |
| 06:00 to 22:00 | | | |
| LS means (95% | 240.96 (182.1, 299.8) | 211.86 (151.5, 272.3) | 180.8 (120.0, 241.6) |
| CI) | | | |
| Difference of LS | 60.2 (−23.3, 143.6) | 31.1 (−53.3, 115.4) | — |
| means (95% CI) | | | |
| p-value vs placebo | 0.16 | 0.47 | — |
| 10:00 to 15:00 | | | |
| LS means (95% | 111.5 (85.4, 137.5) | 100.9 (74.2, 127.6) | 84.7 (57.8, 111.6) |
| CI) | | | |
| Difference of LS | 26.8 (−10.2, 63.7) | 16.2 (−21.1, 53.5) | — |
| means (95% CI) | | | |
| p-value vs placebo | 0.16 | 0.40 | — |

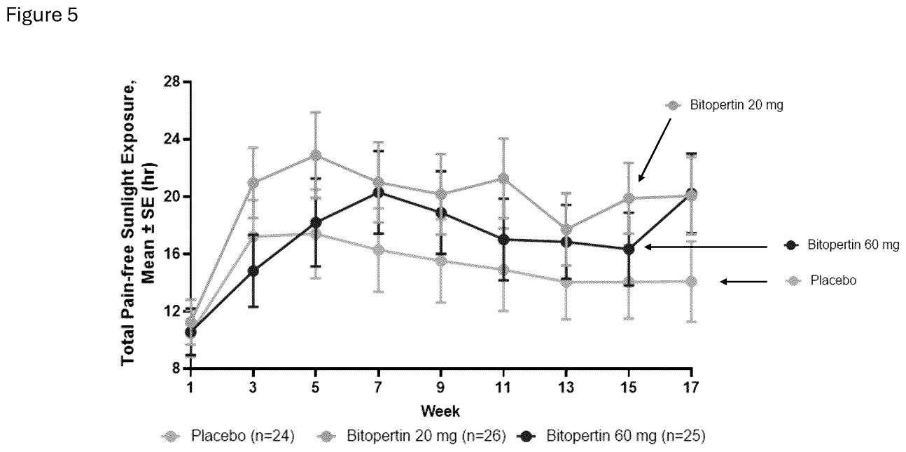

Across all 3 treatment groups, the trajectory of average daily pain free sunlight exposure increased after treatment initiation and continued through Week 5. Thereafter, average sunlight tolerance tended to decrease over time in participants randomized to placebo. In contrast, the initial increase in average sunlight tolerance was mostly maintained through Week 17 in participants randomized to bitopertin. See FIG. 5.

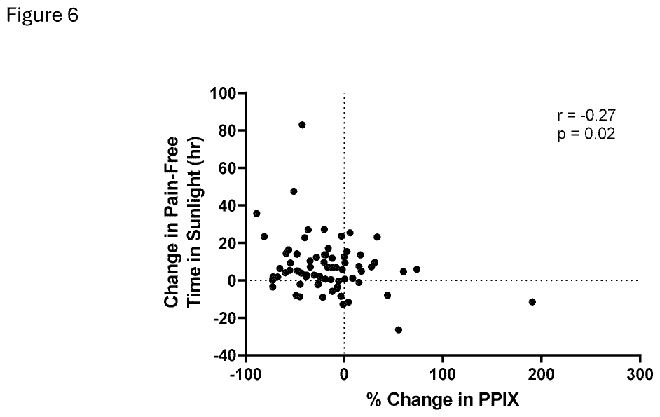

Tertile analyses by subgroups of PPIX change, showed participants with the largest reductions in PPIX had the greatest improvements in sunlight tolerance. See Table 4. For the clinical endpoint of cumulative total pain-free sunlight exposure, participants in Tertile 1 had 40 more hours of cumulative total pain-free sunlight exposure over a 4-month period compared to participants in Tertile 3. PPIX reductions were also associated with improvements in other sunlight tolerance measures, including average daily pain-free time in sunlight, time to first prodromal symptom, occurrence of phototoxic reactions, and improvements in how participants reported feeling, as assessed by the Patient Global Impression of Change (PGIC). Notably, the average total pain-free sunlight exposure observed with 60 mg of bitopertin during the last 2-week interval at the end of study (21.7 hr) represents a 2-fold improvement in average daily sunlight exposure relative to baseline, as assessed during the 2-week screening period (10.7 hr). In contrast, participants randomized to placebo had minimal change in pain-free sunlight exposure at the end of study (13.7 hr) relative to baseline (12.2 hr). These results highlight baseline average daily sunlight tolerance as an important predictor of patient-reported sunlight exposure while also demonstrating that bitopertin offers a significant sunlight tolerance benefit relative to placebo in a time-dependent manner. Reductions in PPIX were correlated with greater improvements in total pain-free time in sunlight during the last 2-week interval of the study relative to baseline. See FIG. 6 and Table 5. Table 5 shows longitudinal analysis of total pain-free time in sunlight exposure averaged over 2-week intervals with baseline daily sunlight tolerance covariate. LS=least squares; MMRM=mixed-model repeated measures; SE=standard error. Least-squares means and p-values for daily sunlight exposure averaged over 2-week intervals were analyzed using MMRM to compare 20 mg and 60 mg bitopertin dose groups versus placebo. MMRM models included fixed effects for treatment, randomization stratification factor, baseline daily light tolerance, visit, treatment-by-visit interactions, and a random effect for each participant. Multiple imputation was used to impute missing data. Baseline average light tolerance represents total sunlight exposure averaged across 14-day screening period, and end-of-study sunlight exposure represents last 2-week interval.

TABLE 5

| Daily Sunlight Tolerance (hr) | Bitopertin, | Bitopertin, | |
|---|---|---|---|
| LS Mean ± SE | 20 mg | 60 mg | Placebo |
| Baseline | 11.1 ± 1.9 | 10.7 ± 1.7 | 12.2 ± 2.1 |
| End of Study | 20.8 ± 2.2 | 21.7 ± 2.3 | 13.7 ± 2.3 |
| End of Study Difference vs | 7.1 ± 3.2 | 8.0 ± 3.2 | — |
| Placebo | | | |
| p-value | 0.026 | 0.013 | — |

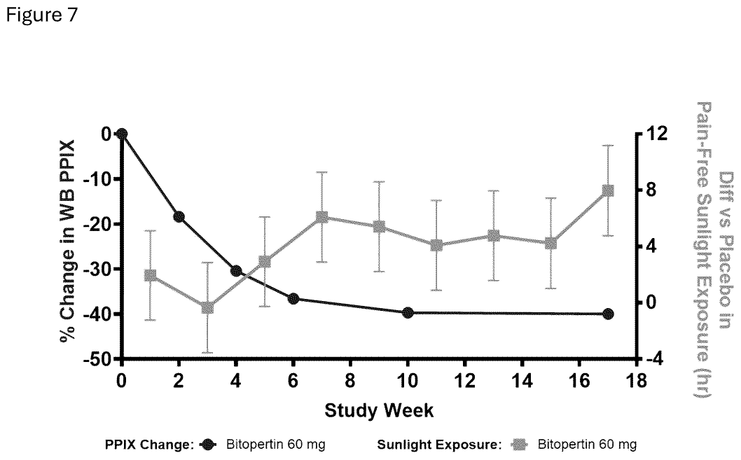

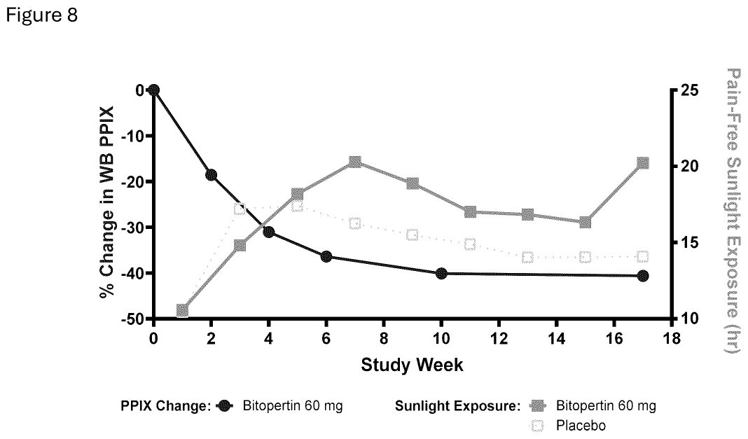

Moreover, the timing for improvements in pain-free sunlight tolerance relative to placebo for participants randomized to 60 mg of bitopertin temporally coincided with the reductions in PPIX levels, which were near maximal 5 to 6 weeks after treatment initiation. See FIG. 7. Separation between bitopertin (60 mg) and placebo groups for pain-free sunlight exposure occurred starting 5 weeks after treatment initiation, which temporally coincided with PPIX reductions >30% in the bitopertin 60-mg dose group. See FIG. 8. Notably, the average total pain-free sunlight exposure observed with 60 mg of bitopertin at the end of the study (20.2 hr during the last 2-week interval) represents a nearly 2-fold improvement relative to baseline average daily sunlight exposure assessed during the 2-week screening period (10.7 hr). In contrast, the initial increase in average sunlight exposure observed in participants randomized to placebo (likely due to expectation bias), was not sustained and waned over time. As a result, pain-free sunlight exposure at the end of the study (14.1 hr) was minimally changed relative to baseline (12.2 hr) for participants randomized to placebo.

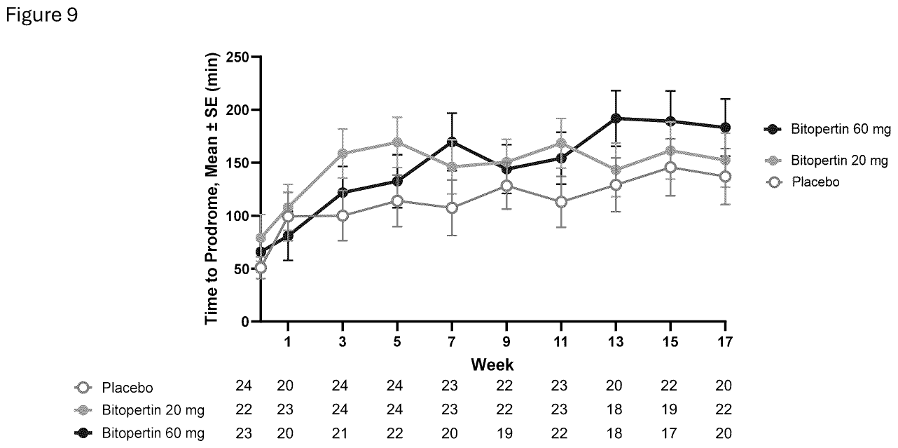

Participants were asked to conduct weekly sun exposure challenges in which participants attempt to elicit a prodrome, or early warning symptoms of a phototoxic reaction such as burning and tingling. See FIG. 9, which shows mean changes from baseline for time to prodrome data averaged over 2-week periods. Improvements in light tolerance relative to baseline were observed in all treatment groups and dose-dependent improvements were observed with bitopertin. In the bitopertin 60-mg group, mean time to prodrome observed at the end of study (183.2 min) represented a nearly 3-fold change from the mean time to prodrome during screening (65.8 min). While the change from baseline in time to prodrome was numerically higher with 60-mg bitopertin compared to placebo at end of study (121.8 min [95% CI 60.7, 183.1] versus 91.9 min [95% CI 33.4, 150.4], respectively), the difference between treatment groups was not statistically significant.

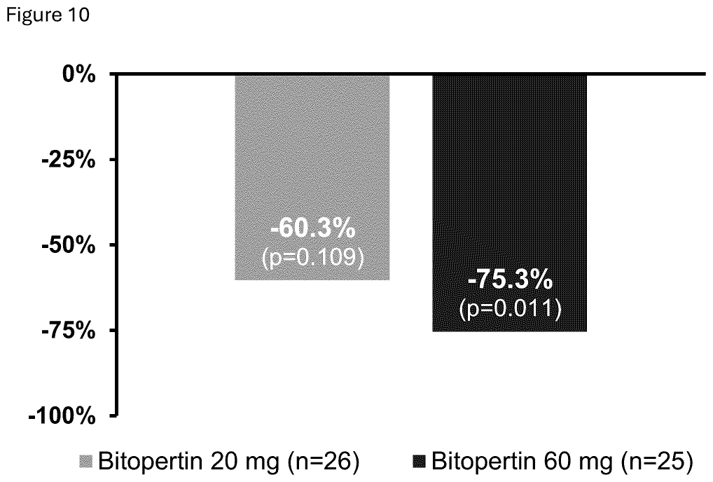

Treatment with bitopertin also resulted in significant, dose-dependent reductions in the rate of phototoxic reactions compared to placebo; the incidence rate ratios relative to placebo were 0.397 (p=0.109) in the 20 mg bitopertin group and 0.247 (p=0.011) in the 60 mg bitopertin group, corresponding to 60% and 75% reductions, respectively, in the rate of phototoxic reactions compared to placebo. See FIG. 10. A total of 11/24 (46%) participants randomized to placebo reported 15 phototoxic reactions during the 17-week double-blind period, compared to 11 phototoxic reactions reported in 5/26 (19%) participants in the 20 mg bitopertin group and 5 phototoxic reactions reported in 3/25 (12%) participants in the 60 mg bitopertin group. See Table 6 which provides a summary of phototoxic reactions and changes in PPIX.

TABLE 6

| | | | | | |
|---|---|---|---|---|---|
| | | Day 1 to | Day 31 to | Day 61 to | Day 91 to |
| Treatment | Screening | Day 30 | Day 60 | Day 90 | Day 121 |

Participants with Phototoxic Reactions, N (%)

| | | | | | |
|---|---|---|---|---|---|
| Placebo | 2 (8%) | 1 (4.2%) | 2 (8.3%) | 4 (16.7%) | 6 (25%) |
| Bitopertin, | 8 (31%) | 4 (15.4%) | 3 (11.5%) | 1 (3.8%) | 1 (3.8%) |
| 20 mg | | | | | |
| Bitopertin, | 6 (24%) | 2 (7.7%) | 2 (7.7%) | 0 (0%) | 0 (0%) |
| 60 mg | | | | | |

Number of Phototoxic Reactions

| | | | | | |
|---|---|---|---|---|---|
| Placebo | 4 | 2 | 2 | 5 | 6 |
| Bitopertin, | 11 | 6 | 3 | 1 | 1 |
| 20 mg | | | | | |
| Bitopertin, | 8 | 2 | 3 | 0 | 0 |
| 60 mg | | | | | |

Percent Change in PPIX (%), Mean ± SE

| | | | | | |
|---|---|---|---|---|---|
| Placebo | — | 6.5 ± 9.9 | 10.1 ± 9.9 | 2.0 ± 8.0 | 9.2 ± 9.6 |
| (n = 24) | | | | | |
| Bitopertin, | — | −22.5 ± 4.5 | −22.3 ± 5.7 | −16.6 ± 9.2 | −19.7 ± 6.6 |
| 20 mg | | | | | |
| (n = 26) | | | | | |
| Bitopertin, | — | −31.7 ± 5.4 | −37.0 ± 6.5 | −40.7 ± 6.7 | −41.3 ± 6.9 |
| 60 mg | | | | | |
| (n = 25) | | | | | |

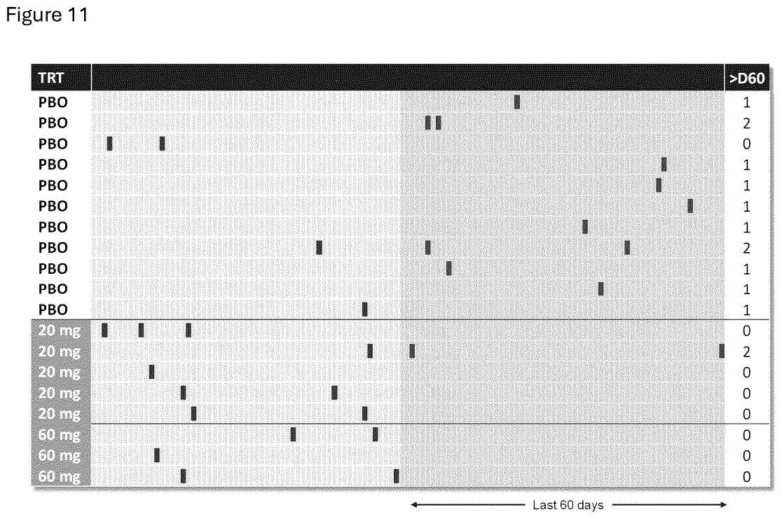

The greatest reduction in the occurrence of phototoxic reactions with bitopertin occurred during the last 60 days of the study, which coincided with the timing for marked PPIX reductions with bitopertin. Although participants randomized to bitopertin reported a higher number of phototoxic reactions during screening as compared to participants randomized to placebo, the occurrence of phototoxic reactions steadily decreased with bitopertin treatment as PPIX levels decreased. See FIG. 11. Notably, after Day 60, almost no phototoxic reactions occurred in participants randomized to bitopertin. In contrast, the number of phototoxic reactions increased during the last 60 days of the study for participants randomized to placebo. In addition to reducing the rate of phototoxic reactions, the median maximum pain score due to phototoxic reactions (as assessed on a numerical rating scale of 1 [least] to 10 [worst]), was also reduced with bitopertin treatment compared to placebo.

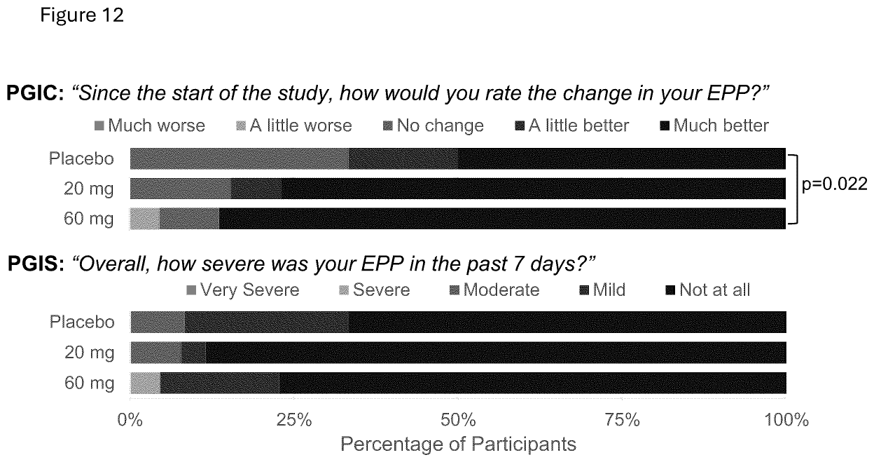

PGIC was also assessed by answering the question “Since the start of the study, how would you rate the change in your EPP?” with one of the following: much better, a little better, no change, a little worse, much worse. Similarly, in the Patient Global Impression of Severity (PGIS), participants responded to “Overall, how severe was your EPP in the past 7 days?” with one of the following: not at all, mild, moderate, severe, very severe. Comparisons between bitopertin treatment groups and placebo for both PGIC and PGIS were analyzed using an ordinal regression model. Bitopertin resulted in improvements in PGIC and Patient Global Impression of Severity (PGIS) compared to placebo, with a greater proportion reporting their EPP was “much better” or “a little better” or was “not at all” severe or “mild” at the end of study. Notably, treatment with bitopertin resulted in dose-dependent improvements in the PGIC, which reached statistical significance in the 60 mg dose group (p=0.022 versus placebo); 77% of participants in the 20 mg bitopertin group and 86% of participants in the 60 mg bitopertin group reported their EPP was “much better” at the end of study compared to 50% in the placebo group. See FIG. 12. Participants in the tertile with the greatest PPIX reductions also experienced fewer phototoxic reactions while PPIX increases were associated with increased occurrence of phototoxic reactions. Amongst participants randomized to bitopertin, greater PPIX reductions were observed at the end of study in participants who reported no phototoxic reaction (−36.5%, n=41) compared to those who reported phototoxic reactions (−4.0%, n=8).

Tertile analyses also showed that PPIX reductions were associated with improved PGIC responses. In the tertile of participants who had the greatest PPIX reductions, a larger proportion of participants reported feeling “Much better” and fewer participants reported “No change” or feeling “A little worse”. See Table 7 which shows Patient Global Impression of Change (PGIC) response by tertiles of PPIX change. Tertile analyses showed that PPIX reductions were associated with improved PGIC responses. In the tertile of participants who had the greatest PPIX reductions, a larger proportion of participants reported feeling “Much better” and fewer participants reported “No change” or feeling “A little worse”.

TABLE 7

| | PPIX Increased | | PPIX Decreased |
|---|---|---|---|
| | [symbol] | | |
| | Tertile 3 | Tertile 2 | Tertile 1 |
| PGIC | (−7% to 190%) | (−38% to −7%) | (−88% to −38%) |
| Response | n = 25 | n = 24 | n = 24 |
| Much better | 12 (48%) | 18 (75%) | 21 (91%) |
| A little better | 5 (20%) | 1 (4%) | 0 |
| No change/A | 8 (32%) | 5 (21%) | 2 (9%) |
| little worse | | | |
| Did not | 0 | 0 | 1 (4%) |
| respond | | | |

Moreover, no major safety concerns (e.g., suicide-related events, dysphoria, depressed mood, blurred vision, abuse liability, and skin disorders) were identified for bitopertin in participants with EPP or XLP at doses up to 60 mg in studies. See Table 8 which shows a summary of safety for the double-blind period. Overall, nearly all participants (95%) who reported a treatment-emergent adverse event (TEAEs) reported events that were mild to moderate in intensity. A total of 3 Grade 3 TEAEs were reported during the double-blind treatment period: 1 occurring in a participant randomized to 60 mg of bitopertin (dizziness) and 2 TEAEs in participants randomized to placebo (obstructive pancreatitis and nephrolithiasis). One SAE of obstructive pancreatitis was reported in a participant randomized to placebo during the double-blind treatment period. One participant randomized to 60 mg of bitopertin discontinued treatment due to a TEAE (drug eruption), and 1 additional participant randomized to 60 mg of bitopertin discontinued due to iron deficiency anemia (which occurred prior to dosing and was not treatment-emergent).

TABLE 8

| | Placebo | Bitopertin | Bitopertin |
|---|---|---|---|
| | (n = 24) | 20 mg (n = 26) | 60 mg (n = 25) |
| Participants with any | 18 (75%) | 20 (76.9%) | 22 (88%) |
| TEAE, n (%) | | | |
| TEAEs leading to | 0 | 0 | 1 (4%) |
| discontinuation, n (%) | | | |
| Grade 3 TEAEs, n (%) | 2 (8.3%) | 0 | 1 (4%) |
| Serious adverse events, | 1 (4.2%) | 0 | 0 |
| n (%) | | | |

The most commonly reported TEAEs during the double-blind treatment period are shown in Table 9, nearly all of which were mild to moderate in intensity. Specifically, Table 9 shows the most commonly reported TEAEs during the double-blind treatment period. Nearly all of which were mild to moderate in intensity (other than the Grade 3 events noted above). TEAEs of dizziness were transient, with median duration of 1 to 5 days, and severity of the TEAEs was dose-related, with Grade 2 events reported in 6 (23.1%), 9 (36%), and 2 (8.3%) participants randomized to the 20 mg bitopertin, 60 mg bitopertin, and placebo groups, respectively.

TABLE 9

| | Placebo | Bitopertin | Bitopertin |
|---|---|---|---|
| Preferred Term | (n = 24) | 20 mg (n = 26) | 60 mg (n = 25) |
| Dizziness, n (%) | 4 (16.7) | 3 (11.5) | 11 (44) |
| Median duration (days) | 2 | 1 | 5 |
| Nausea, n (%) | 2 (8.3) | 1 (3.8) | 4 (16) |
| Alanine aminotransferase | 3 (12.5) | 1 (3.8) | 2 (8) |
| increased, n (%) | | | |

This study demonstrated that the profile of average pain-free sunlight exposure with bitopertin is distinct from that of afamelanotide. Afamelanotide, a subcutaneous implant, is administered every 2 months. Therefore, the pattern of average pain-free sunlight exposure over time with afamelanotide is influenced by the timing of implant administration, with some waning of treatment effect over each 2-month cycle prior to the next successive implant administration. In contrast, the profile of pain-free sunlight exposure with bitopertin, which is taken orally, QD, shows time-dependent treatment effects that are mostly maintained with continued daily administration. In summary this study demonstrated, for both doses tested, but with greater improvements across multiple efficacy measures, including increased time to prodrome, increased pain-free time in sunlight at the end of study (last 2-week interval), reductions in phototoxic reactions and associated pain, as well as improvements in PGIC. See Table 10, which provides a summary of results from additional prespecified efficacy endpoints comparing bitopertin to placebo. The observed efficacy across multiple measures provides evidence that in addition to significant reductions in PPIX, bitopertin can also improve how patients with EPP feel and function.

TABLE 10

| | | Bitopertin, 20 mg vs | | Bitopertin, 60 mg |
|---|---|---|---|---|
| Endpoint | Assessment | Placebo | | vs Placebo |
| Primary | Change in whole-blood metal-free | −29.7% (p = 0.004) | | −48.7% (p < 0.001) |
| | PPIX (%) | | | |
| Key | Cumulative total pain-free time in | 41.2 | hr | 19.2 hr |
| Secondary | sunlight (hr) | | | |
| Secondary | Change in time to first prodromal | −18.2 | min | 30.0 min |
| | symptom (min) | | | |
| | Maximum pain intensity score of | −1.0 | | −1.5 |
| | phototoxic reactions (median) | | | |
| | Total pain intensity score of | −3.0 | | −4.5 |
| | phototoxic reactions (median) | | | |
| | Change in erythrocyte PPIX (%) | −28.6% (p = 0.006) | | −50.0% (p < 0.001) |
| | Change in plasma PPIX (%) | −28.7% (p = 0.006) | | −50.7% (p < 0.001) |
| | Change in whole-blood PPIX (%) | −29.9% (p = 0.004) | | −51.2% (p < 0.001) |
| Exploratory | Incidence rate ratio of phototoxic | 0.397 | | 0.247 (p = 0.011) |
| | reactions | | | |
| | PGIC (odds ratio) | 3.1 | | 5.2 (p = 0.022) |
| | PGIS (odds ratio) | 3.1 | | 1.7 |
| | Change in time to prodrome after | 91.3 | min | 64.1 min |

| | | |
|---|---|---|
| Week 5 (min) | | |
| PROMIS-57 Pain Measurement | −4.4 | −4.0 |
| Domain | | |
| (change in mean T-score) | | |
| PROMIS Social Isolation | −2.4 | −0.2 |
| (mean T-score) | | |
| PROMIS Neuropathic Pain Quality | −0.2 | −0.8 |
| (mean T-score) | | |

### Example 4: Pharmacodynamics of Bitopertin in Subjects with EPP

Data from clinical pharmacology studies have demonstrated the glycine uptake (GlyT1) inhibition potential of bitopertin in RBCs from healthy subjects (e.g., no confirmed diagnosis of EPP or XLP). The clinical evidence of the downstream pharmacodynamic (PD) effect of glycine inhibition, demonstrating the mechanism of action of bitopertin on hematologic parameters, such as Hgb, MCH, and MCV, comes from a Phase 1 healthy volunteer safety hematology study. See Table 11 which shows pharmacokinetic (PK) parameters determined in several repeat-dose studies. Bitopertin reached peak plasma concentrations after around 4 hours. At steady state, bitopertin exposure (AUC [0-τ] and Cmax) increased in a dose-proportional manner across the dose range tested, i.e., up to 180 mg/day. Ninety percent of steady-state trough concentrations were reached between 6 and 8 days. The AUC (0-τ) increased from Day 1 to steady state about 2.7 to 3.8-fold with a tendency for greater accumulation with higher doses. Bitopertin has a low systemic clearance, with CL/F between 4.35 and 5.32 L/h. Overall, the half-life estimate of bitopertin is around 2 days.

Bitopertin was shown to inhibit glycine uptake (GlyT1 inhibitor) in a dose-dependent manner at doses ranging from 24 to 180 mg, with a near maximal effect achieved at a 60 mg dose exposure. In addition, in clinical studies of EPP, treatment with bitopertin results in significant, dose-dependent reductions in PPIX levels, with more pronounced PPIX reductions observed with 60 mg than with 20 mg of bitopertin.

Inhibition of GlyT1 and modulation of heme synthesis is also mirrored by dose-dependent reductions in mean corpuscular hemoglobin and mean corpuscular volume in red blood cells, as observed in EPP clinical studies. None of the changes were associated with any clinical symptoms, and these pharmacological effects were shown to be reversible in other non-EPP clinical studies. Additionally, mean hemoglobin levels remained unchanged relative to baseline with bitopertin in EPP. While some patients experienced small decreases in hemoglobin, none were considered clinically significant or associated with anemia. Mild, dose-dependent increases in serum iron that were not clinically significant have also been observed with bitopertin treatment.

Cardiac Electrophysiology: In a thorough QT (TQT) study, there was no clinically relevant increase in QTc interval after multiple administrations of bitopertin at doses up to 175 mg per day (2.9 times the maximum recommended human dose).

TABLE 11

| | | | | Cₘₐₓ | C₂₄ | AUC_{(0-τ)} | |
|---|---|---|---|---|---|---|---|
| | | | | (ng/mL) | (ng/mL) | (ng × hr/mL) | t_{1/2} (hr) |
| | | | Tₘₐₓ (hr) | GM | GM | GM | GM |
| | | | Median | (CV % | (CV % | (CV % | (CV % |
| Dose (mg) | Study | N | (Range) | GM) | GM) | GM) | GM) |
| 10 | 1028094 | 14 | 4.00 (2.00- | 123 | 55.7 | 1800 | 54.8 |
| | (BP21441) | | 6.00) | (37.6) | (60.8) | (52.9) | (35.7) |
| | 4 Month ^{a} | | | | | | |
| 30 | 1028094 | 14 | 4.00 (2.00- | 411 | 198 | 6660 | 49.0 |
| | (BP21441) | | 6.00) | (39.0) | (67.1) | (51.0) | (41.1) |
| | 4 Month ^{a} | | | | | | |
| | 1031728 | 56 | 3.29 (1.12- | 408 | 236 | 7180 | 53.5 |
| | (BP21705) | | 8.17) | (24.0) | (36.2) | (30.0) | (34.4) |
| | TQT ^{b} | | | | | | |
| 60 | 1028094 | 12 | 4.00 (2.00- | 851 | 442 | 14200 | 64.3 |
| | (BP21441) | | 6.00) | (33.1) | (58.1) | (47.5) | (44.0) |
| | 4 Month ^{a} | | | | | | |
| 175 | 1031728 | 52 | 4.25 (2.17- | 1690 | 1200 | 33400 | 53.0 |
| | (BP21705) | | 12.38) | (28.0) | (32.8) | (30.0) | (37.8) |
| | TQT ^{b} | | | | | | |

In this study, Hgb decreased dose-dependently following daily oral administration of 10, 30, or 60 mg over 120 days. MCH and MCV in reticulocytes as well as MCH and MCV in RBCs decreased dose-dependently. For the reticulocyte parameters, a plateau was reached after 15 days. The RBC parameters decreased continuously without reaching an obvious plateau at or before 120 days treatment. The pharmacological effects observed in these studies were all reversible after treatment cessation.

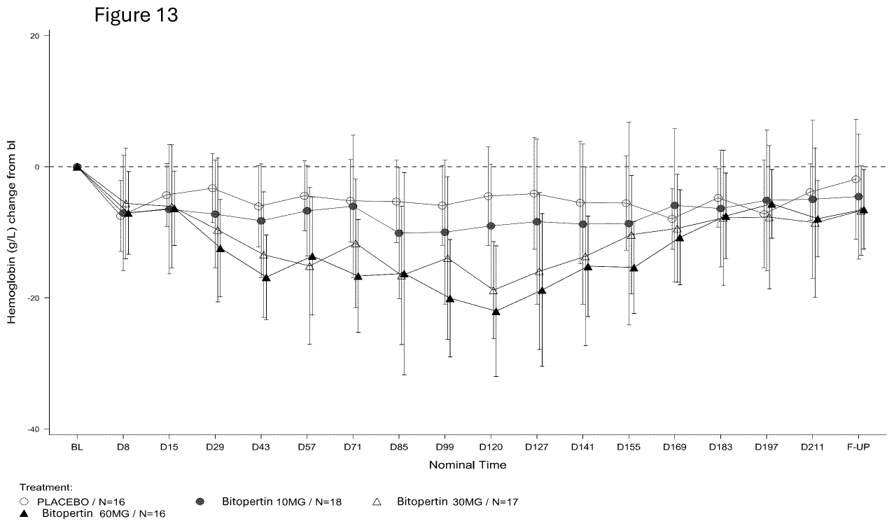

Placebo, 10, 30, or 60 mg bitopertin was administered QD for 120 days. See FIG. 13. The decrease in RBC parameters variable and overall comparable between placebo and the 10 mg group as well as between 30 mg and 60 mg groups. On Day 120, the estimated difference to placebo (g/L) with 90% CIs amounted to −4.5 (−10.7 to 1.8), −13.4 (−19.7 to −7.2), and −18.5 (−24.9 to −12.2) after 10, 30, and 60 mg bitopertin, respectively. In the 30 mg and 60 mg dose groups, Hgb was estimated to be 9.3% (90% CI −13.5, −5.0) and 13.0% (90% CI −17.0, −8.7) lower than in the placebo group. No participant met the discontinuation criterion of the study, i.e., confirmed Hgb value <100 g/L in females and <110 g/L in males. Differences between treatment groups started to emerge after 15 days of treatment.

MCH and MCV in reticulocytes as well as MCH and MCV in RBCs decreased in a dose-dependent manner. Data not shown. For the reticulocyte parameters, a plateau was reached after 15 days. The RBC parameters decreased continuously without reaching an obvious plateau at or before Day 120 of treatment. In almost all participants in the 60 mg dose group and several participants in the 30 mg dose group, the RBC parameters fell below the normal range. In general, there was no obvious gender difference, and all changes were reversible. No parameter reached a level at which clinical signs or symptoms would be expected.

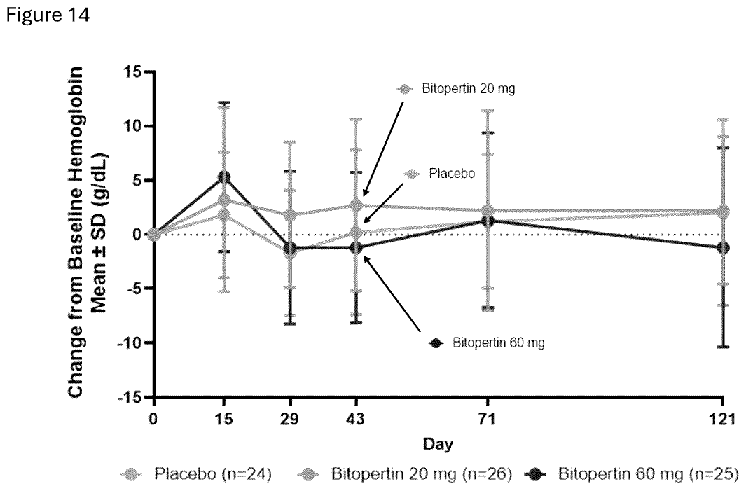

Consistent with the murine EPP model (Example 2), in participants with EPP (Example 3), mean hemoglobin levels remained unchanged relative to baseline with daily bitopertin treatment (both 20 mg and 60 mg doses). See FIG. 14. While some participants experienced small decreases in hemoglobin, none were considered clinically significant. Moreover, there were no anemia TEAEs in this study, and only 1 participant receiving 20 mg bitopertin reported anemia, which was classified as Grade 1 (mild) in severity and unlikely related to bitopertin.

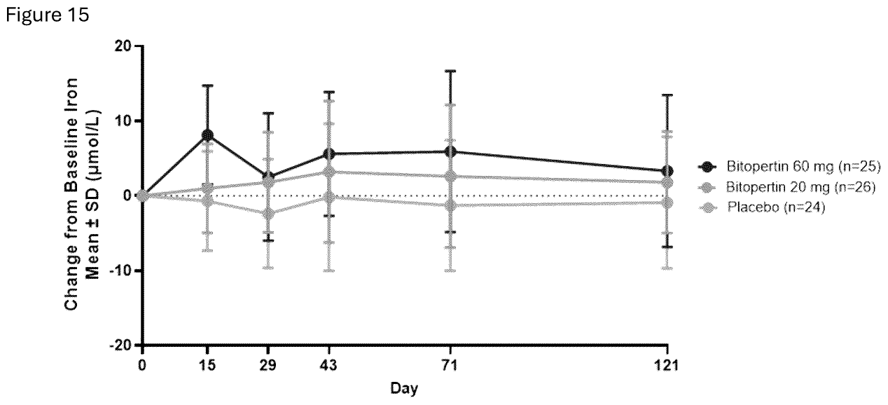

In addition, mild, dose-dependent increases in serum iron were observed with bitopertin treatment that were not clinically significant. See FIG. 15.

Bitopertin exposure increased in a linear and dose-proportional manner following single doses over the dose range of 12 mg to 120 mg (0.2 to 2 times the recommended dose) and multiple doses up to 180 mg (3 times the recommended dose). Steady-state plasma concentrations were attained within 6 to 8 days of once-daily dosing and the accumulation ratios of maximum concentration (Cₘₐₓ) and AUC₀₋₂₄ were 2.42 and 3.44, respectively, for bitopertin 60 mg once-daily dosing.

Bitopertin was readily absorbed after single and multiple dose administration both in healthy subjects and patients with EPP. Median Tₘₐₓ values for the 60 mg dose administered under fasted and fed conditions were 4 and 5 hours, respectively. Administration of a single 60 mg dose of bitopertin tablet with a high-fat/high-caloric meal was associated with an increase of AUC_{0-∞} by 16.8% and Cₘₐₓ by 31.6% relative to the exposure achieved when given under fasted condition.

Bitopertin is highly protein bound in human plasma (98%), primarily to serum albumin. The overall volume of distribution from the population pharmacokinetic analysis of bitopertin studies was estimated as 316 L. Bitopertin has been shown to cross the placenta in rats and is present in the milk of lactating rats.

The mean elimination half-life (t_{1/2}) of bitopertin estimated from population pharmacokinetic analysis was 49 hours following multiple-dose administration of 10 mg to 60 mg once daily in healthy subjects. Population pharmacokinetics-derived median apparent clearance at steady state was estimated at 4.81 L/h.

In vitro and clinical pharmacology studies showed that bitopertin is primarily metabolized by CYP3A4. After a single dose oral administration of 80 mg of 14C-bitopertin, 68% (8-22% as unchanged bitopertin) of the dose was recovered in feces and 18% (<0.2% unchanged bitopertin) in urine, indicating metabolic clearance as the major elimination pathway for bitopertin. In population pharmacokinetic analysis using adult subjects, no clinically meaningful effects on the pharmacokinetics of bitopertin were observed based on age (13-75 years), sex, race (White/Other, Black, and Asian), or body weight (44.5-130 kg). Dose-normalized exposure in adolescent patients with EPP was similar to the adult group.

The data described in these examples demonstrate the ability of bitopertin to reduce heme biosynthesis in a dose-dependent manner through reduced availability of intracellular glycine, but not hematologic parameters, such as Hgb in subjects with EEP or XLP.

### Example 5: Phase 2 Study in Adults and Adolescents with Erythropoietic Protoporphyria (EPP) or X-linked Protoporphyria (XLP)

This was a Phase 2, randomized, open-label, parallel-arm study in adult and adolescent participants with EPP or XLP. Participants received oral, once-daily administration of 20 mg or 60 mg of bitopertin for 24 weeks. Twenty-six participants were randomized to 20 mg (n=14) or 60 mg (n=12) of bitopertin. The study met its primary efficacy endpoint; treatment with bitopertin resulted in significant, dose-dependent reductions in PPIX levels compared to baseline at both the 20 mg and 60 mg dose levels (−31.7±7.0% [p<0.001 vs baseline] and −57.7±7.4% [p<0.001 vs baseline], respectively). Similar efficacy was observed across adult and adolescent populations. No serious adverse events were reported. Dizziness was the most common adverse event reported with bitopertin (6 [55%] in 20-mg and 7 [64%] in 60-mg dose groups). By reducing whole-blood PPIX levels, bitopertin targets the underlying pathophysiology of EPP, resulting in consistent improvements in multiple measures of light tolerance and quality of life. Bitopertin was well tolerated and most adverse events were mild or moderate in severity.

### Study Design

This was a Phase 2, randomized, open-label, parallel-arm study in individuals with EPP or XLP who received oral, once-daily (QD) administration of 20 mg or 60 mg of bitopertin for 24 weeks. The study initially recruited adults with a confirmed diagnosis of EPP or XLP. A subsequent protocol amendment allowed the enrollment of adolescent participants ≥12 to <18 years of age. The starting dose for adolescents was 50% of their randomized treatment assignment. On Day 15, adolescent participants dose-escalated to receive the randomized dose of 20 mg or 60 mg QD. A formal sample size calculation was not performed; as the first study of bitopertin in EPP/XLP, a sample size of 10 participants per group completing 24 weeks of treatment (assuming 11 participants enrolled with 1 early treatment discontinuation per cohort) was considered appropriate to assess the initial safety and tolerability in this population.

### Study Population

The study included adults and adolescents with a confirmed diagnosis of EPP or XLP by porphyrin analysis and/or genetic testing. Exclusion criteria included aspartate aminotransferase/alanine aminotransferase values ≥2× the upper limit of normal, total bilirubin >upper limit of normal, hemoglobin <10 g/dL, or concurrent treatment with afamelanotide or the investigational therapy, dersimelagon.

### Randomization and Intervention

Randomization was stratified by site and baseline light tolerance (time to prodrome <30 or ≥30 minutes), as assessed over a 2-week period in screening. Upon completion of the 24-week treatment period, participants were given the option to continue bitopertin treatment in an open-label extension study.

### Endpoints

The primary endpoint was percent change from baseline in whole-blood metal-free PPIX. The key secondary endpoint was the cumulative total hours of sunlight exposure between 10:00 AM to 6:00 PM on days with no pain from a phototoxic reaction. Additional secondary and exploratory endpoints include patient-reported outcomes of light tolerance and quality of life, safety, and tolerability. See Table 12.

TABLE 12
Prespecified Efficacy Endpoints

| | |
|---|---|
| Primary | Percent change from baseline in whole-blood metal-free |
| Endpoint | protoporphyrin IX (PPIX) at Week 24 (Day 169) |
| Key Secondary | Total hours of sunlight exposure to skin on days with no pain |
| Endpoint | from 10:00 to 18:00 hours (10:00 AM to 6:00 PM) from |
| | randomization to Day 169 |
| Secondary | Change in 2-week average daily sunlight exposure time |
| Endpoints | (minutes) to first prodromal symptom (e.g., burning, |
| | tingling, itching, or stinging) associated with sunlight |
| | exposure between 1 hour post-sunrise and 1 hour pre-sunset |
| | through Day 169 |
| | Pain intensity of phototoxic reactions according to a Likert |
| | scale (0-10) |
| | Safety and tolerability of bitopertin, as assessed by the |
| | incidence of treatment-emergent adverse events |
| | Erythrocyte metal-free PPIX concentrations through Day 169 |
| | Plasma and whole-blood total PPIX concentrations through Day 169 |
| | Plasma bitopertin concentrations |
| Exploratory | Number of phototoxic reactions with a pain component |
| Endpoints | (recorded in a diary) through Day 169 |
| | Time to recovery of phototoxic reactions with a pain component |
| | Change in time to prodromal symptom in sunlight on a |
| | single day, assessed once weekly, from Week 5 through Day 169 |
| | Changes from baseline in daily light dosimetry measurements |
| | Change from baseline in patient-reported outcome (PRO) |
| | scores from: Patient-Reported Outcomes Measurement |
| | Information System (PROMIS)-57 V2.1; PROMIS-Short |
| | Form v2.0 - Social Isolation 8a; PROMIS Neuropathic Pain |
| | Quality v2.0; PROMIS v2.0 Pediatric Profile 49 |
| | (adolescents only); EPP Questionnaire |
| | Change in Patient Global Impression of Change (PGIC) scores |
| | Change in Patient Global Impression of Severity (PGIS) scores |
| | Change from baseline in liver injury biomarkers, including |
| | aspartate aminotransferase, alanine aminotransferase, total |
| | bilirubin, and alkaline phosphatase |
| | Change from baseline of steatosis and fibrosis results from |
| | liver FibroScan or acoustic radiation force impulse (ARFI) |
| | ultrasound results |
| | Correlation of bitopertin PK and PPIX response |

### Assessments

Participants recorded daily sunlight exposure, presence and severity of symptoms, and the occurrence of phototoxic reactions in an electronic daily sunlight exposure diary. The development and content validation of the sunlight exposure diary have been previously described (Mathias, 2023). Data collected in the diary were used in the analysis of the key secondary and other efficacy endpoints. The study also included weekly sun exposure challenges, in which participants attempt to elicit a prodrome, or early warning symptoms of a phototoxic reaction such as burning and tingling. Participants recorded the total amount of time they were able to spend in the sunlight before experiencing early warning symptoms. Laboratory measurements including biomarkers for liver injury (alanine aminotransferase, aspartate aminotransferase, total bilirubin, and alkaline phosphatase) and hematology including hemoglobin were collected during screening, Day 1, and throughout the 24-week treatment period. Additional assessments included FibroScan or acoustic radiation force impulse ultrasound assessments at baseline and end of study to assess liver fibrosis and steatosis. Safety was monitored by documenting all adverse events and monitoring laboratory results and vital sign measurements.

### Statistical Analysis

A mixed-model repeated-measures (MMRM) approach was used to analyze the primary endpoint, with percent change from baseline in whole-blood metal-free PPIX through Week 24 as the response, and fixed effects for baseline light tolerance strata, baseline whole-blood metal-free PPIX, treatment group, visit, and interactions between treatment group and visit. The model included all measurements collected through Week 24, irrespective of study drug administration (i.e., intention-to-treat principle). Missing PPIX data were not imputed. For key secondary endpoint, we compared the total hours of sunlight exposure on days with no pain from 10:00 AM to 6:00 PM summed over the entire treatment period from randomization to Week 24 using analysis of variance, with fixed effects for randomized treatment group and baseline light tolerance strata. We imputed missing sunlight exposure diary data using non-missing sunlight diary entries on days with no pain from a phototoxic reaction.

### Results

Twenty-two adults were randomized, 11 to receive 20 mg of bitopertin and 11 to receive 60 mg of bitopertin; additionally, 4 adolescents were randomized, 3 to receive 20 mg of bitopertin and 1 to receive 60 mg of bitopertin. A total of 21 of 22 adult participants and 3 of 4 adolescent participants completed the 6-month treatment period. Two participants randomized to the 20 mg bitopertin group discontinued treatment early due to a treatment-emergent adverse event (TEAE). Demographics and select baseline characteristics of participants randomized into the study are shown in Table 13 and were generally well balanced between the 20 mg and 60 mg bitopertin groups. One participant with XLP was randomized into the study.

TABLE 13
Demographics and Select Baseline Disease Characteristics of Study Participants

| | Adult Population | | Adolescent Population | |
|---|---|---|---|---|
| | Bitopertin | Bitopertin | Bitopertin | Bitopertin |
| Characteristic | 20 mg (n = 11) | 60 mg (n = 11) | 20 mg (n = 3) | 60 mg (n = 1) |
| Mean age, years | 43.2 | 44.5 | 14.3 | 14.0 |
| Female, n (%) | 6 (55%) | 8 (73%) | 2 (67%) | 1 (100%) |
| Race, n (%) | | | | |
| White | 11 (100%) | 10 (91%) | 3 (100%) | 1 (100%) |
| Asian | 0 | 1 (9%) | 0 | 0 |
| Hispanic or Latino, n (%) | 0 | 0 | 0 | 0 |
| EPP, n (%) | 11 (100%) | 10 (91%) | 3 (100%) | 1 (100%) |
| XLP, n (%) | 0 | 1 (9%) | 0 | 0 |
| Baseline PPIX, | 11920 ± 2260 | 8560 ± 2006 | 4537 ± 719 | 3570 |
| Mean ± SE (ng/mL) | | | | |
| Time to prodrome, n (%) | | | | |
| <30 min | 7 (64%) | 6 (55%) | 0 | 0 |
| ≥30 min | 4 (36%) | 5 (45%) | 3 (100%) | 1 (100%) |
| Total pain-free time in | 7.8 ± 7.6 | 7.5 ± 8.8 | 14.7 ± 10.3 | 2.7 |
| sunlight, mean ± SD (hr)^{a} | | | | |

### Efficacy

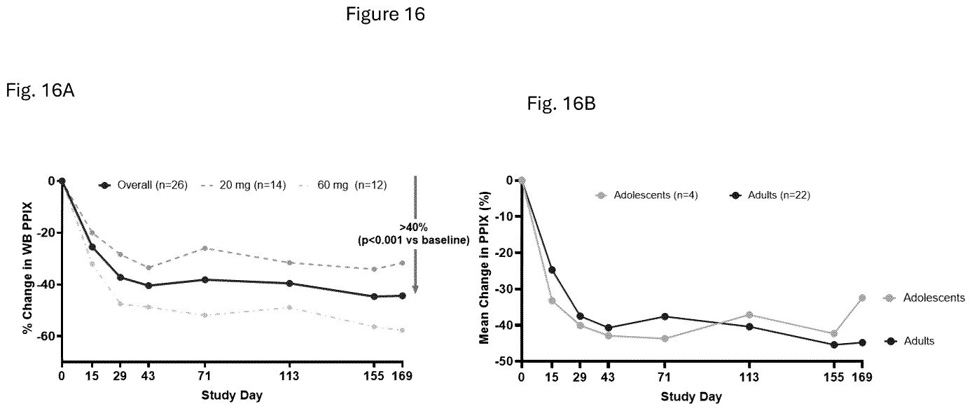

For the primary endpoint, treatment with bitopertin resulted in significant and sustained reductions in PPIX levels compared to baseline at both the 20 mg and 60 mg dose levels. See FIG. 16A. The reductions were dose dependent with more pronounced decreases observed with 60 mg (LS Mean±SE: −57.7±7.4% [p<0.001 vs baseline]) compared to 20 mg (LS Mean±SE: −31.7±7.0% [p<0.001 versus baseline]). See Table 14. The reductions in PPIX with bitopertin were observed across many prespecified subgroups, including sex, age, EPP and XLP subtypes, and measures of disease severity, as assessed by baseline PPIX levels (<median or >median for the randomized ITT population) and baseline light tolerance (time to prodrome <30 minutes or ≥30 minutes). Treatment with bitopertin also resulted in meaningful reductions in PPIX levels in the adolescent population, with mean (±SE) reductions of −32.4±14.8% relative to baseline at the end of study. See FIG. 16B.

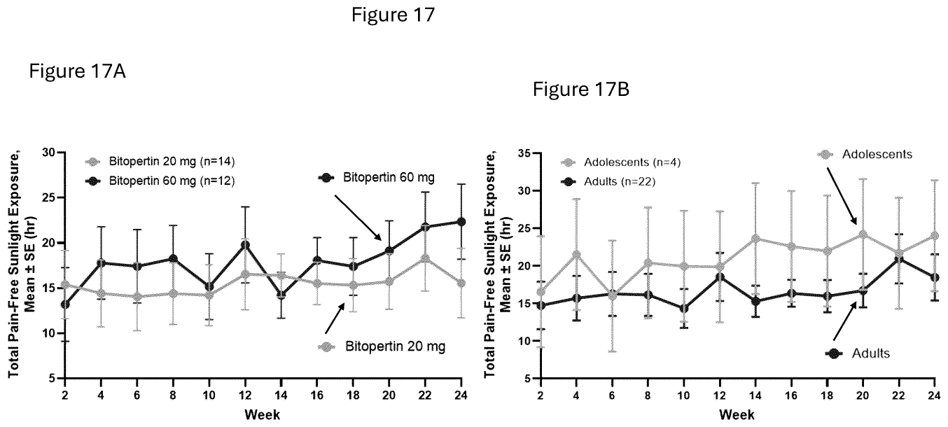

The key secondary endpoint showed a dose-dependent increase in the cumulative total time in light without pain observed over a 6-month treatment period, with a mean of 212.1 hours in the 60 mg dose group compared to 180.0 hours in the 20 mg dose group. The adolescent subpopulation had a numerically higher cumulative pain-free time in sunlight over the 6-month treatment period compared to the adult subpopulation (LS mean±SE: 254.1±74.3 hour versus 196.3±26.5 hour, respectively). A post hoc MMRM analysis of total pain-free daily sunlight exposure averaged over 2-week intervals showed time-dependent improvements through Week 24 with the 60 mg dose of bitopertin groups. See FIG. 17A. The average total pain-free sunlight exposure observed with 60 mg of bitopertin during the last 2-week interval at the end of study represented a 1.8-fold improvement in average daily sunlight exposure relative to baseline, as assessed during the 2-week screening period. Similar time-dependent improvements in pain-free time in sunlight were observed in the adolescent populations. See FIG. 17B.

TABLE 14
Primary and Key Secondary Efficacy Endpoints

| | Adults (n = 22) | | Adolescents (n = 4) | | Overall (n = 26) | |
|---|---|---|---|---|---|---|
| | Bitopertin | Bitopertin | Bitopertin | Bitopertin | Bitopertin | Bitopertin |
| | 20 mg | 60 mg | 20 mg | 60 mg | 20 mg | 60 mg |
| Endpoint | (n = 11) | (n = 11) | (n = 3) | (n = 1) | (n = 14) | (n = 12) |

Primary endpoint: percent change from baseline in whole-blood metal-free PPIX at Week 24

| | | | | | | |
|---|---|---|---|---|---|---|
| Least-squares | −27.8 | −61.3 | −42.4 | — | −31.7 | −57.7 |
| mean | (7.4) | (7.4) | (9.3) | | (7.0) | (7.4) |
| (SE), % | | | | | | |
| P -value vs | 0.001 | <0.001 | 0.002 | — | <0.001 | <0.001 |
| baseline | | | | | | |

Key secondary endpoint: cumulative total no. hours in sunlight between 10 AM and 6 PM
without pain from a phototoxic reaction through Week 24

| | | | | | | |
|---|---|---|---|---|---|---|
| Least-squares | 180.0 | 212.1 | 259.8 | 237.2 | 186.0 | 210.0 |
| mean (SE), hr | (38.2) | (37.6) | (104.7) | (181.4) | (33.5) | (36.2) |

MMRM = mixed-model repeated measures;
PPIX = protoporphyrin IX;
WB = whole blood
Primary endpoint: least-squares means results for percent change in WB metal-free PPIX levels obtained from MMRM analysis. MMRM models included fixed effects for randomization stratification factor (time to prodrome <30 min or ≥30 min), baseline PPIX, treatment, visit, treatment-by-visit interactions, and a random effect for each participant. P-values calculated by comparing WB metal-free PPIX levels at Day 169 relative to baseline. The 60 mg adolescent participant had a missing Day 169 PPIX sample and missing data were not imputed.
Secondary endpoint: Least-squares means for cumulative time in light measured via daily diary, adding all time in light between the hours of 10:00 am and 6:00 pm on days without any pain from a phototoxic reaction, and analyzed using an analysis of variance model.

### Exploratory Outcomes

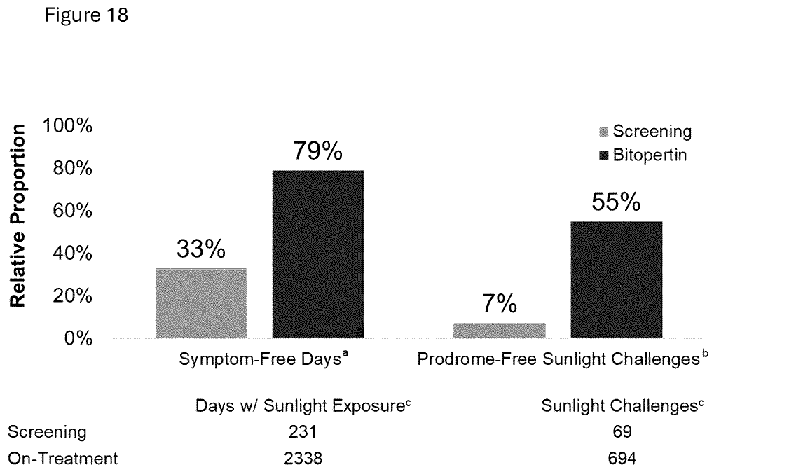

The proportion of prodrome-free sunlight challenges for adults increased from 7% during screening to 55% while receiving bitopertin (n=22), and the proportion of days without symptoms (with sun exposure) increased from 33% during screening to 79% while receiving bitopertin (n=22). See FIG. 18. Patient-reported phototoxic reactions decreased by 92% while on treatment compared to baseline; participants reported phototoxic reactions on 27/231 (11.7%) days with sunlight exposure during screening compared to 22/2338 (0.9%) while receiving bitopertin. Quality of life at Week 24, as assessed with the patient global impression of change (PGIC), also improved with bitopertin treatment. Nearly all adult participants (95%) reported in the PGIC their EPP was much better or a little better, 90% reported in the patient global impression of severity (PGIS) their EPP was mild or not at all severe at the end of the study (compared to 62% at baseline), and 86% reported their EPP had a little or no impact on their overall quality of life after 6 months of treatment with bitopertin (compared to only 29% at baseline). Prespecified tertile analyses by subgroups of PPIX change showed participants with the largest reductions in PPIX had the greatest improvements in measures of sunlight tolerance, including cumulative or average daily sunlight exposure and time to first prodromal symptom.

TABLE 15
Light Tolerance Measures by Tertiles of PPIX Change

| | Tertile 3 | Tertile 2 | Tertile 1 |
|---|---|---|---|
| Light Tolerance Measure | (−43% to 25%) | (−53% to −43%) | (−98% to −53%) |
| (Mean ± SD) | (n = 9) | (n = 9) | (n = 8) |
| Cumulative total time in | 145.6 ± 99.5 | 190.5 ± 111.6 | 262.1 ± 170.6 |
| light without pain (hr) | | | |
| Average daily time in light | 0.9 ± 0.6 | 1.1 ± 0.7 | 1.6 ± 1.0 |
| without pain (hr) | | | |
| Change from baseline in | 85.3 ± 78.8 | 96.0 ± 109.0 | 165.5 ± 128.8 |
| time to prodrome (min) | | | |

Abbreviations: PPIX, protoporphyrin X. PPIX decreases from tertile 3 to tertile 1.

### Safety

A total of 20/22 (91%) adult participants reported a treatment-emergent adverse event. One adult participant randomized to 20 mg of bitopertin discontinued treatment early due to an adverse event (cluster headache), which was the only Grade 3 adverse event reported in the study. All other adverse events were mild to moderate in intensity. No serious adverse events were reported in the study. In the adolescent population, a total of 9 adverse events were reported in 4 (100%) participants, all of which were mild in severity. One adolescent participant (20 mg) discontinued treatment because of an adverse event of suicidal ideation. The participant had a medical history of depressed mood and suicidal ideation that was not reported during screening and given the participant's medical history, the Sponsor recommended that the participant withdraw study medication. The most common adverse events, defined as those reported in more than 2 participants, are shown in Table 16. Overall, dizziness was the most commonly reported adverse event with bitopertin and was reported similarly across the 20 mg and 60 mg dose groups. Dizziness events were transient, with nearly all events (95%) occurring in the first month of the study and median duration of 5 days. No meaningful trends were observed in the aggregate clinical laboratory data in either the adult or adolescent populations, including hematology parameters and vital signs.

TABLE 16
Summary of Adverse Events (Safety Population)

| | Adult Population | | Adolescent Population | |
|---|---|---|---|---|
| | Bitopertin | Bitopertin | Bitopertin | Bitopertin |
| | 20 mg (n = 11) | 60 mg (n = 11) | 20 mg (n = 3) | 60 mg (n = 1) |
| Any adverse events, n (%) | 9 (82%) | 11 (100%) | 3 (100%) | 1 (100%) |
| Adverse events leading to | 1 (9%) | 0 | 1 (33%) | 0 |
| discontinuation, n (%) | | | | |
| Grade 3 adverse events, n (%) | 1 (9%) | 0 | 0 | 0 |
| Serious adverse events, n (%) | 0 | 0 | 0 | 0 |
| Common adverse events occurring | | | | |
| in >2 participants | | | | |
| Dizziness, n (%) | 6 (55%) | 7 (64%) | 3 (100%) | 1 (100%) |
| Headache, n (%) | 3 (27%) | 1 (9%) | 0 | 0 |
| Nausea, n (%) | 1 (9%) | 2 (18%) | 0 | 0 |

Results from this Phase 2, open-label study demonstrated that treatment with bitopertin was safe and led to significant, dose-dependent decreases in PPIX in adults and adolescents with EPP or XLP. The time course of PPIX levels showed sustained reductions through the 24-week treatment period, and the nadir was achieved 4 to 6 weeks after initiation of bitopertin treatment. The reductions in PPIX with bitopertin were associated with improvements in multiple measures of sunlight tolerance. For the key secondary endpoint, the cumulative total pain-free time in sunlight over the 6-month treatment period was higher in the 60 mg dose group compared to the 20 mg dose group, and also numerically higher in the adolescent subpopulation compared to the adult population. Notably, the cumulative total time in light observed in this study was approximately 3 times higher than that previously reported in the placebo arm of the Phase 3 afamelanotide study over the same 6-month period (60.6 hours; Langendonk J G, Balwani M, Anderson K E, Bonkovsky H L, Anstey A V, Bissell D M, et al. Afamelanotide for erythropoietic protoporphyria. N Engl J Med 2015; 373 (1): 48-59). Additionally, a post hoc longitudinal analysis showed total pain-free sunlight tolerance increased relative to baseline over time for participants randomized to 60 mg bitopertin. The average total pain-free sunlight exposure observed with 60 mg of bitopertin at the end of study depicted in FIGS. 17A and B represents a greater than 2-fold improvement relative to baseline average total sunlight exposure assessed during the 2-week screening period. See Table 15. A similar 2-fold improvement relative to baseline was also observed in the adolescent subpopulation.

The safety profile of bitopertin in this study was generally consistent with that observed in prior studies. Bitopertin was previously studied by Roche and no significant safety concerns were identified in prior clinical studies that exposed more than 4000 participants in other indications (including schizophrenia). Prior clinical studies and population pharmacokinetics modeling with bitopertin showed no age-related effects in drug exposure and safety assessments. Because bitopertin is cleared primarily by metabolism via cytochrome P450 (CYP)3A4, and CYP3A4 expression is known to be matured in early childhood (Eidelman, 2016), the similarity in bitopertin exposure across adult age groups was extrapolated to the adolescent population and adolescent participants in the study received the same bitopertin doses as adult participants. Consistent with the expected similarity of drug exposure and pharmacodynamic effects between adolescent and adult populations, this study demonstrated a similar favorable safety profile across adult and adolescent EPP/XLP populations. Results from this study also indicate that adolescent patients derive the same benefits of PPIX reduction and improved light tolerance with bitopertin as adults. Therefore, bitopertin in certain embodiments is used as a treatment in adolescent patients with EPP, a subpopulation for whom there are no available therapies.

The most common adverse events reported with bitopertin in this study were dizziness, all of which were mild to moderate in severity and resolved within several days. No serious adverse events were reported in the study. No meaningful trends were observed in the clinical laboratory data, including hemoglobin levels, indicating that the restriction of glycine availability with bitopertin does not impede the necessary heme required for hemoglobin biosynthesis. Results from this Phase 2 study support the effectiveness and safety of bitopertin as an oral treatment for reducing PPIX and increasing light tolerance in adults and adolescent patients with EPP or XLP.

### Example 6: Phase 3, Multicenter, Randomized, Double-Blind, Placebo-Controlled Study Evaluating the Efficacy, Safety, and Tolerability of Bitopertin in the Treatment of Erythropoietic Protoporphyria (EPP) or X-Linked Protoporphyria (XLP)

Painful phototoxicity, the hallmark of EPP, is caused by accumulation of PPIX in the skin and has negative impacts on patients' daily function and quality of life. For some patients with EPP, phototoxic reactions occur within minutes of exposure to sunlight. Pain from phototoxic reactions is intense, generally lasts for several days, and typically does not respond to even the strongest analgesics, including narcotics. As a result, many patients resort to spending days in darkness and isolation waiting for the pain from phototoxic reactions to subside. There remains an unmet need for therapies in EPP that can reduce disease-causing PPIX elevations, and there are currently no approved therapies for patients <18 years of age. Provided herein in certain embodiments, are methods for the treatment of EPP or XLP comprising an effective does of bitopertin for 6 months or more that reduce phototoxicity and/or improve sunlight tolerance in a human being.

Collective data from clinical studies in EPP showed the following with bitopertin:
Significant, sustained, dose-dependent reductions in PPIX compared to placebo
Consistent association between reductions in PPIX and improvements in clinical outcome measures related to sunlight tolerance
Time-dependent improvements in pain-free sunlight tolerance, with 2-fold increase relative to baseline
Dose-dependent reductions in the rate of phototoxic reactions and associated pain compared to placebo
Dose-dependent improvements in PGIC compared to placebo

Provided herein is a study demonstrating the treatment of EPP or XLP comprising administering bitopertin to a subject in need thereof, wherein the treatment improves the average monthly total time in sunlight on days without pain from a phototoxic reaction between 10:00 to 18:00 (10:00 AM to 6:00 PM) after 6 months (24 weeks) of treatment and/or reduces from baseline in whole-blood metal-free PPIX levels at 6 months. In certain embodiments, the treatment further reduces the occurrence of phototoxic reactions over the 6-month treatment period; increases the cumulative total time in sunlight on days without pain from a phototoxic reaction between 10:00 to 18:00 (10:00 AM to 6:00 PM) over the 6-month (24-week) treatment period and/or increase from baseline in 2-week average daily sunlight exposure time (minutes) to first prodromal symptom (e.g., burning, tingling, itching, or stinging) associated with sunlight exposure between 1 hour post-sunrise and 1 hour pre-sunset at 6 months. In certain other embodiments, the treatment improves one or more of PGIC scores at 6 months; Patient Global Impression of Severity scores at 6 months; changes in patient-reported outcomes (PROs) at 6 months; and/or time to first event over the 6-month treatment period in the composite endpoint (e.g., 10% increase from baseline in PPIX, or Phototoxic reaction).

### Study Design

This is a Phase 3, multicenter, double-blind, placebo-controlled, parallel-group study of bitopertin to evaluate the efficacy, safety, and tolerability in participants with EPP or XLP. Participants will be screened for study eligibility within 28 days before Day 1 and for at least 16 days. All participants will undergo light tolerance assessment during screening (Sun Exposure Challenge Question and Sun Exposure Diary). The Sun Exposure Challenge is optional for adolescents.

Approximately 150 participants aged 12 years and older are planned to be enrolled. Participants who are determined to be eligible based on screening assessments will be randomized on Day 1 in a 1:1 ratio to receive bitopertin (n=~75) or placebo (n=~75) in the following treatment groups: Placebo (n=~75) administered as 2 tablets or Bitopertin 60 mg (n=~75) administered as 2×30 mg tablets. EPP diagnosis can be made by genetic (FECH or ALAS2) or biochemical porphyrin analysis. EPP is a disease caused by congenital defects in either of the heme biosynthesis pathway enzymes FECH (autosomal recessive EPP) or ALAS2 (X-linked EPP; XLP). Defects in either of these enzymes result in toxic accumulation of PPIX. Because glycine is required in the initial step of heme biosynthesis, the decrease in glycine uptake via GlyT1 inhibition with bitopertin can reduce downstream heme synthesis intermediates, including PPIX arising from either FECH or ALAS2 defects in EPP.

Randomization will be stratified by sunlight exposure time to prodromal symptom (<30 minutes or ≥30 minutes), as assessed during a 2-week period in screening, and geographical region. Daily diary compliance will also be evaluated during this screening period.

On Day 1, participants will begin study drug according to their randomized treatment assignment and will be evaluated during a 6-month (24-week) treatment period thereafter. Approximately 4 weeks after the End of Study (EOS) visit, participants who do not enter the separate open-label extension study will have a safety follow-up visit. Participants will maintain a daily Sun Exposure Diary throughout the study.

If any participant becomes intolerant of study drug, a dose reduction of 50% is permitted after consultation with the Investigator and Sponsor. The starting dose for adolescents will be 50% of their randomized treatment assignment. At Day 15 (+2 days) and thereafter, the full dose may be administered at the Investigator's discretion. A Data Monitoring Committee (DMC) will evaluate safety data throughout the study.

After completion of the 6-month treatment period, including assessment of light tolerance, and EOS visit, participants may be eligible to continue bitopertin in a separate open-label extension study. See FIG. 16.

### Study Objectives and Endpoints

For participants with erythropoietic protoporphyria (EPP) or X-linked protoporphyria (XLP) randomized in this study, the objectives comparing treatment with bitopertin relative to placebo are as follows:

### Primary:

To assess average pain-free sunlight exposure tolerance after 6 months of treatment.
To assess changes in whole-blood metal-free protoporphyrin IX (PPIX) levels after 6 months of treatment.
To assess safety and tolerability.

### Secondary:

To assess the occurrence of phototoxic reactions.
To assess cumulative total pain-free sunlight exposure over 6 months of treatment.
To assess changes in time to prodromal symptoms.

### Exploratory:

To assess Patient Global Impression of Change (PGIC).
To assess changes in patient-reported changes in disease severity.
To assess changes in patient-reported quality of life measures.
To assess percentage of participants with a clinical worsening event in EPP, defined as increase in PPIX or occurrence of a phototoxic reaction.
To assess the relationship between changes in whole-blood metal-free PPIX with changes in EPP-related signs and symptoms.

The primary endpoint of this study will compare the bitopertin and placebo groups with respect to the average monthly time in sunlight without pain between 10:00 and 18:00 after 6 months of treatment, and therefore is designed to capture improvements in pain-free light tolerance over time. See Table 17 which shows the efficacy endpoints for the Phase 3 study.

TABLE 17

| | |
|---|---|
| Primary | Average monthly total time in sunlight on days without pain |
| Endpoint | from a phototoxic reaction between 10:00 to 18:00 (10:00 AM |
| | to 6:00 PM) after 6 months (24 weeks) of treatment |
| | Percent change from baseline in whole-blood metal-free PPIX |
| | levels at 6 months |
| | Safety and tolerability, as assessed by adverse events (AEs) |
| | and laboratory results, over the 6-month treatment period |
| Secondary | Occurrence of phototoxic reactions over the 6-month |
| Endpoints | treatment period |
| | Cumulative total time in sunlight on days without pain from a |
| | phototoxic reaction between 10:00 to 18:00 (10:00 AM to |
| | 6:00 PM) over the 6-month (24-week) treatment period |
| | Change from baseline in 2-week average daily sunlight |
| | exposure time (minutes) to first prodromal symptom (eg, |
| | burning, tingling, itching, or stinging) associated with sunlight |
| | exposure between 1 hour post-sunrise and 1 hour pre-sunset at |
| | 6 months |
| Exploratory | PGIC scores at 6 months |
| Endpoints | Patient Global Impression of Severity scores at 6 months |
| | Changes in patient-reported outcomes (PROs) at 6 months |
| | Time to first event over the 6-month treatment period in the |
| | composite endpoint consisting of: |
| | 10% increase from baseline in PPIX, or |
| | Phototoxic reaction |
| | Correlations between changes in whole-blood metal-free |
| | PPIX with other efficacy endpoints at 6 months |

In other words, the primary endpoint comparing the difference between treatment groups for average monthly “total time in sunlight without pain between 10:00 and 18:00” after 6 months is a clinically meaningful endpoint in EPP studies that is well suited for evaluating time-dependent improvements in sunlight tolerance seen with bitopertin. The comparison will use a longitudinal analysis to evaluate time-dependent treatment effects on average pain-free sunlight exposure. For each participant, the sum of the total hours of sunlight exposure on days with no pain from phototoxic reactions will be calculated by month. A mixed-model repeated-measures (MMRM) analysis will include all daily sunlight exposure diary data accrued over the 6-month treatment period and will compare the difference in pain-free sunlight exposure during the last month of the 6-month treatment period for participants randomized to bitopertin compared to participants randomized to placebo.

The proposed primary endpoint is a PRO-based endpoint that will leverage the same sunlight exposure diary previously used in the Example 3 secondary endpoint. Briefly, study participants will access the sunlight exposure diary once daily through an electronic portal. The total time spent in sunlight between 10:00 to 18:00 will be summed over monthly intervals throughout the 6-month study. The analysis will exclude days on which respondents indicate they experienced pain from a phototoxic reaction. Because the amount of time spent in sunlight can vary greatly from day to day due to weather and scheduled activities, the total hours of time spent in sunlight without pain summed over the course of monthly intervals was chosen to reduce some of the variability inherent in the data.

If the diary is not completed on a given day or if reactions to sunlight exposure (full reaction or no symptoms) are missing, the missing daily daylight tolerance will be imputed under a missing at random (MAR) assumption. Additional prespecified sensitivity analyses will explore alternate handling methods for missing data, including no imputation, control-based imputation, and tipping point analyses to support the MAR assumption.

For the co-primary efficacy endpoint of average time in sunlight on days without pain from a phototoxic reaction between 10:00 to 18:00 (10:00 AM to 6:00 PM) during the last month of the 6-month (24 week) treatment period, mixed-model repeated-measures (MMRM) analysis will be used to compare the difference in pain-free sunlight exposure during the last month of the 6-month treatment period for participants randomized to bitopertin compared to participants randomized to placebo. The treatment effect will be estimated using the difference between the treatment LS means (adjusted means). The LS means difference with corresponding 95% confidence interval will be presented along with the p-value from the hypothesis test of no difference between the treatment groups.

The co-primary endpoint evaluating percent changes in whole-blood metal-free PPIX will be analyzed using a mixed-model repeated measures (MMRM) with the ITT population. The dependent variable is the percent change from baseline whole-blood metal-free PPIX level for all post-baseline assessments for each participant. The model will include fixed effects for treatment, randomization stratification factor, baseline whole-blood metal-free PPIX level, visit, and visit-by-treatment interaction and a random effect for the participant. The co-primary endpoints will each be tested with α=0.05. The primary endpoints will also be analyzed for subgroups of participants aged <18 years and ≥18 years and for participants with EPP or XLP.

Secondary efficacy analyses include the occurrence of phototoxic reactions over the 6-month treatment period (using a negative binomial regression model); cumulative total pain-free time in sunlight on days without a phototoxic reaction between 10:00 to 18:00 (10:00 AM to 6:00 PM) over the 6-month (24-week) treatment period (using an analysis of variance (ANOVA) model, and change from baseline in 2-week average daily exposure time to first prodromal symptom (using an MMRM analysis). Secondary endpoints will be analyzed using a hierarchical approach to maintain the family-wise overall Type I error rate of 0.05. Nominal p-values will be reported for secondary endpoints that are not analyzed under the hierarchical testing strategy. Formal testing of secondary endpoints in the hierarchy may proceed so long as statistically significant evidence of benefit continues to be shown. Comparisons between bitopertin and placebo groups for PGIC (1st in the hierarchy) will be performed using an ordinal regression model. Data from the study of Example 3 showed that treatment with 60 mg of bitopertin significantly improved the PGIC (p=0.022), which asks patients to assess how their EPP has improved or worsened relative to the start of treatment. The improvements in PGIC were also associated with reductions in PPIX. Accordingly, this study will assess PGIC as a secondary endpoint to further assess how patients with EPP feel with bitopertin treatment.

The incidence rate ratio of phototoxic reactions (2nd secondary endpoint) will evaluate the effect of bitopertin on phototoxic reactions. Between-group comparisons of the number of phototoxic reactions will be performed using a negative binomial regression model. Phototoxic reactions represent a severe, clinical manifestation that has the greatest impact on the daily function of patients. For some patients with EPP, phototoxic reactions occur within minutes of exposure to sunlight and result in extreme pain and visible skin alterations. The pain is intense, implacable, generally lasts for several days, and typically does not respond to even the strongest analgesics, including narcotics. Because of the intractable pain, many patients resort to spending days in darkness and isolation waiting for the pain from phototoxic reactions to subside.

This study will evaluate the cumulative total hours of sunlight exposure to skin on days with no pain from 10:00 to 18:00 after 6 months of treatment as a secondary endpoint (3rd in the hierarchy). The analysis of this endpoint will use the same daily sunlight diary data used in the analysis of the proposed primary endpoint for this study. The secondary endpoint results using an analysis of the cumulative total time in sunlight will support the findings of the primary endpoint analysis. This study will also evaluate the percent change in PPIX for participants randomized to bitopertin compared to participants randomized to placebo as a secondary endpoint. Evaluation of PPIX changes will provide additional mechanistic rationale for bitopertin and confirmatory evidence that bitopertin can target the underlying pathophysiology of EPP.

Secondary endpoints will be analyzed using a hierarchical approach to maintain the family-wise overall Type I error rate of 0.05. Nominal p-values will be reported for secondary endpoints that are not analyzed under the hierarchical testing strategy. Formal testing of secondary endpoints in the hierarchy may proceed so long as statistically significant evidence of benefit continues to be shown. Details will be described in the SAP.

The secondary endpoints will also be analyzed for subgroups of participants aged <18 years and >18 years and for participants with EPP or XLP. Additional subgroup analyses may be performed and will be described in the SAP.

### Inclusion Criteria

Participants are eligible for the study if all of the following criteria apply:
Aged 12 years or older at the time of study consent.
Diagnosis of EPP or XLP, based on medical history by ferrochelatase (FECH) or aminolevulinic acid synthase 2 (ALAS2) genotyping or by biochemical porphyrin analysis.
Minimum daily Sun Exposure Diary compliance ≥85% on Days −14 through Day −1, inclusive, during screening, and at least 1 successfully completed Sun Exposure Challenge (adults only, as this assessment is optional for adolescents) or historical recall of time to prodrome.
Body weight ≥32 kg (ages 12 to <18 years), body mass index ≥18.5 kg/m² (ages ≥18 years) at screening.
Washout of at least 2 months prior to screening of afamelanotide and dersimelagon, if applicable.
Aspartate aminotransferase and alanine transaminase <3× upper limit of normal (ULN) and total bilirubin <2×ULN (unless documented Gilbert syndrome) at screening. Albumin >lower limit of normal (LLN).
Willing to practice highly effective methods of birth control (both males who have partners of childbearing potential and females of childbearing potential during screening, while taking study drug, and for at least 30 days after the last dose of study drug.
Negative pregnancy test (females of childbearing potential) at screening (Days −28 to −1) AND baseline (Day 1), prior to dosing.
Able to understand the study aims, procedures, and requirements, and provide written informed consent (and assent if necessary).
Able to comply with all study procedures.

### Exclusion Criteria

Participants are excluded from the study if any of the following criteria apply: Medical History:
1. Major surgery within 8 weeks before screening or incomplete recovery from any previous surgery.
2. Other than EPP or XLP, an inherited intrinsic or extrinsic red cell disease associated with anemia, eg, G6PD, hemoglobinopathy, membranopathy, or immune or nonimmune hemolytic anemia. Any comorbid hematologic disease must be deemed acceptable by the Sponsor.
3. Known hypersensitivity to any component of the study drug.
4. History of liver transplantation or anticipated need for liver transplantation.
5 History of alcohol dependence or excessive alcohol consumption, as assessed by the Investigator.
6. Active human immunodeficiency virus (HIV), active hepatitis B or C. A positive HIV or viral hepatitis test result should be discussed between the Investigator and Sponsor prior to enrollment.
7. Score of Personal Health Questionnaire Depression Scale (PHQ-8) ≥10 at screening or imminent suicidal risk identified by the Columbia-Suicide Severity Scale (C-SSRS) as defined as suicidal ideation with intent (Grade 4 or 5) within the last year or any suicidal behavior within the last 5 years.
8. Other medical or psychiatric condition or laboratory finding not specifically noted above that, in the judgment of the Investigator or Sponsor, would put the participant at unacceptable risk or otherwise preclude the participant from participating in the study.
9. Condition or concomitant medication that would confound the ability to interpret clinical, clinical laboratory, or participant diary data, including a major psychiatric condition that has had an exacerbation or required hospitalization in the last 6 months.

### Treatment History

1. Prior exposure to bitopertin.
2. Concurrent or planned treatment with afamelanotide or dersimelagon during the study period.
3. Treatment with opioids for any period >7 days in the 2 months prior to screening or anticipated to require opioid use for >7 days at any point during the study.
4. New treatment for anemia, including initiation of iron supplementation, within 1 month of screening.
5. Current or planned use of any drugs or herbal remedies known to be strong or moderate inhibitors or inducers of cytochrome P450 (CYP)3A4 enzymes for 28 days prior to the first dose and throughout the study.
6. Current or planned treatment with antipsychotic medication.

### Laboratory Exclusions:

Hemoglobin <10 g/dL at screening.

### Miscellaneous:

Participation in other interventional clinical studies within 30 days prior to screening.
If female, pregnant or breastfeeding.

### Statistical Methods

Approximately 150 participants will be randomized and allocated in a 1:1 ratio to receive bitopertin (n=~75) or placebo (n=~75).

One co-primary efficacy endpoint of this study is average time in sunlight on days without pain from a phototoxic reaction between 10:00 to 18:00 (10:00 AM to 6:00 PM) during the last month of the 6-month (24-week) treatment period. Mixed-model repeated-measures (MMRM) analysis will be used to compare the difference in pain-free sunlight exposure during the last month of the 6-month treatment period for participants randomized to bitopertin compared to participants randomized to placebo. Assuming the difference in means between bitopertin and placebo is 11 hours with a corresponding pooled standard deviation of 24 hours, a sample size of approximately 75 participants per group will provide 80% power to detect a difference between the 2 treatment groups using a 2-sample t-test with a 2-sided alpha=0.05. Note that the treatment effect assumption of 11 hours conservatively accounts for 8% of the study population discontinuing treatment and having no change. Moreover, assuming a discontinuation rate of 8% and a pooled standard deviation of 42, a sample size of approximately 75 participants per group will provide over 80% power to detect a difference between the two treatment groups in percent change from baseline in PPIX of 21% using a 2-sample t-test with a 2-sided alpha-0.05. The treatment effect will be estimated using the difference between the treatments least-squares (LS) means (adjusted means). The LS means difference with corresponding 95% confidence interval will be presented along with the p-value from the hypothesis test of no difference between the treatment groups.

The other co-primary endpoint of percent change in whole-blood metal-free PPIX level will be analyzed using MMRM analysis with the ITT analysis set. The dependent variable is the percent change from baseline whole-blood metal-free PPIX level for all post-baseline assessments for each participant.

The co-primary endpoints will each be tested with α=0.05. The primary and secondary endpoints will also be analyzed for subgroups of participants aged <18 years and ≥18 years and for participants with EPP or XLP.

The study will have 1 formal blinded sample-size recalculation due to uncertainty associated with the assumptions used (notably, variability) in the sample size calculation. Using blinded information, the aggregate (blinded) variance in average daily sunlight exposure observed over monthly intervals will be calculated and used to compute the estimated power under the originally assumed treatment effect. Based on the results obtained from this recalculation, the study size will be increased, if necessary. This interim analysis will be conducted after the first 50 participants have completed the 6-month study period. As no unblinding will occur and treatment effect differences will not be known or assessed, the recalculation will not have a discernible impact on the Type I error rate for the final analysis.

A comprehensive Statistical Analysis Plan (SAP) will be developed detailing statistical methodologies and outlining all planned analyses.

Participants will fast overnight (minimum 6 hours) prior to blood draws. Water is allowed. Blood draws should occur pre-dose (except for post-dose PK sampling), and at the same time of day ±2 hours at each scheduled assessment. Scheduled assessment times for blood chemistry and hematology will be at all study visits except for Day 2 and Day 15. For urinalysis, collection will occur at all study visits except screening, Day 2, and Day 15, prior to dosing. Blood samples will be assessed for: Blood chemistry: blood glucose, sodium, potassium, blood urea nitrogen (BUN), creatinine, AST, ALT, alkaline phosphatase, total bilirubin, direct bilirubin, gamma glutamyl transferase (GGT), albumin, calcium, phosphorus, carbon dioxide (CO₂), and chloride; Hematology: Hgb, hematocrit, RBC count, red-cell distribution width, WBC count with differential, platelet count, MCV, mean corpuscular hemoglobin (MCH), and mean corpuscular hemoglobin concentration (MCHC); Special hematology: reticulocyte (absolute), reticulocyte %, and CHr; and, Urinalysis: color, pH, specific gravity, protein, blood, glucose, nitrites, and ketones. Urine sediment microscopy will be conducted in the instance of abnormal findings for blood.

Metal-free whole-blood PPIX will be measured at screening and all visits. Blood for iron studies will be collected in the fasted state (>6 hours), between 6:00 AM and noon. Collection will occur at baseline (Day 1) and on Days 85 and 169 (EOS). The iron parameters to be assessed are: transferrin, TIBC, serum iron, TSAT, and ferritin. Blood sampling for PK analyses will occur on Day 1 (pre-dose, 2±0.25, and 4±0.25 hours post-dose), Day 2 (24±1 hours post-Day 1 dose, but prior to Day 2 dosing), Day 29 (pre-dose and 2±0.5 hours post-dose), Day 43 (pre-dose), and Day 85 (pre-dose). The date and time of the dose taken on the day prior to PK blood collection day (Days 29, 43, and 85) will be recorded.

All participants will undergo light-tolerance assessment during screening (Sun Exposure Diary and Sun Exposure Challenge Question). Daily Sun Exposure Diary is to be completed for at least 2 weeks during screening and daily during the double-blind treatment phase. The Sun Exposure Challenge (time to prodrome) is to be completed to the point of prodromal symptoms twice during screening, if possible, and weekly thereafter. The Sun Exposure Challenge is optional for adolescents. In the event that both challenges cannot be completed, the participant's recall of their typical time to prodrome during sun exposure shall be recorded during screening, including adolescents. Participants will expose their skin to sunlight once a week and measure the time it takes to experience a prodrome. The time (minutes) to first prodromal symptom (eg, burning, tingling, itching, or stinging) associated with sunlight exposure will be recorded in a diary.

The EPP Impact Questionnaire (EPIQ) is a Sponsor-developed exploratory tool that includes QOL measures. Items related to the PGIC and the Patient Global Impression of Severity (PGIS) will be assessed as part of the EPIQ. The EPIQ will be assessed at Days 1, 43, 85, 113, 169/EOS, and at 4-Week Safety Follow-Up.

For the co-primary efficacy endpoint of average time in sunlight on days without pain from a phototoxic reaction between 10:00 to 18:00 (10:00 AM to 6:00 PM) during the last month of the 6-month (24 week) treatment period, mixed-model repeated-measures (MMRM) analysis will be used to compare the difference in pain-free sunlight exposure during the last month of the 6-month treatment period for participants randomized to bitopertin compared to participants randomized to placebo. The treatment effect will be estimated using the difference between the treatment LS means (adjusted means). The LS means difference with corresponding 95% confidence interval will be presented along with the p-value from the hypothesis test of no difference between the treatment groups. If the diary is not completed on a given day or if reactions to sunlight exposure (full reaction or no symptoms) are missing, the missing daily daylight tolerance will be imputed under a missing at random (MAR) assumption. Additional prespecified sensitivity analyses will explore alternate handling methods for missing data, including no imputation, control-based imputation, and tipping-point analyses to support the MAR assumption.

The co-primary endpoint evaluating percent changes in whole-blood metal-free PPIX will be analyzed using a mixed-model repeated measures (MMRM) with the intent to treat (ITT) population. The dependent variable is the percent change from baseline whole-blood metal-free PPIX level for all post-baseline assessments for each participant. The model will include fixed effects for treatment, randomization stratification factor, baseline whole-blood metal-free PPIX level, visit, and visit-by-treatment interaction and a random effect for the participant. The co-primary endpoints will each be tested with α=0.05. The primary endpoints will also be analyzed for subgroups of participants aged <18 years and ≥18 years and for participants with EPP or XLP.

Secondary efficacy analyses include the occurrence of phototoxic reactions over the 6-month treatment period (using a negative binomial regression model); cumulative total pain-free time in sunlight on days without a phototoxic reaction between 10:00 to 18:00 (10:00 AM to 6:00 PM) over the 6-month (24-week) treatment period (using an analysis of variance (ANOVA) model, and change from baseline in 2-week average daily exposure time to first prodromal symptom (using an MMRM analysis). Secondary endpoints will be analyzed using a hierarchical approach to maintain the family-wise overall Type I error rate of 0.05. Nominal p-values will be reported for secondary endpoints that are not analyzed under the hierarchical testing strategy. Formal testing of secondary endpoints in the hierarchy may proceed so long as statistically significant evidence of benefit continues to be shown. Details will be described in the SAP. The secondary endpoints will also be analyzed for subgroups of participants aged <18 years and ≥18 years and for participants with EPP or XLP. Additional subgroup analyses may be performed and will be described in the SAP.

Pharmacokinetic variables include trough plasma study-drug levels as well as PK parameters, which will be listed and summarized by dose group using descriptive statistics. Additional pharmacokinetic analysis and relationships of drug exposure to clinical effects will be evaluated using a separate PK-PD analysis protocol.

The effect of Hgb reduction with bitopertin is anticipated to be attenuated in individuals with EPP or XLP because excess PPIX substrate should always be available for heme synthesis, even in the context of reduced flux of glycine in the heme synthesis pathway due to GlyT1 inhibition. Bitopertin may cause mild decreases in hemoglobin, although anemia is not expected in EPP based on cell and animal model data of EPP, as well as clinical studies in EPP. Nevertheless, hematology parameters will be monitored and evaluated in clinical studies of EPP or XLP and DBA, and dose reduction may be considered for significant decreases in Hgb levels.

### Example 7: Bitopertin in the Treatment of Hepatopathy

Liver disease affects approximately 5-20% of patients with erythropoietic protoporphyria (EPP) and can progress to cirrhosis and fulminant hepatic failure. EPP hepatopathy is associated with the pathologic elevation protoporphyrin IX (PPIX). Hepatobiliary accumulation of PPIX leads to gallstones, hepatic inflammation, and fibrosis. The only approved therapy for EPP (afamelanotide; Scenesse) does not lower PPIX levels and is not expected to impact the development or progression of liver disease. By contrast, the GlyT1 inhibitor bitopertin directly reduces systemic PPIX levels and therefore may reduce risk of this co-morbidity. Indeed, in vivo data confirm bitopertin's ability to prevent liver fibrosis and cholestasis in a murine EPP model.

The HELIOS trial is an ongoing open-label long term extension study that follows EPP and XLP patients 12 years and older who were previously enrolled in the AURORA, BEACON, and APOLLO studies. HELIOS collects data on long term safety, tolerability, and efficacy of bitopertin at two dose levels (i.e., 20 mg or 60 mg once daily). Approximately 200 patients will be enrolled. The primary endpoints are the incidence of treatment emergent adverse events and safety data for up to 5 years. Secondary endpoints include changes in light tolerance, whole blood metal-free PPIX levels, and markers of liver disease. The soluble liver biomarkers (ALT, AST, AP, and GGT) are collected as part of clinical chemistry at day 1, weeks 8,16, 24, 52, and then every 6 months. Exploratory hepatobiliary markers include serum bile acid salts and AP isoenzymes and are collected at day 1, week 24, week 52, and every 6 months thereafter. Liver imaging by FibroScan or acoustic radiation force impulse (ARFI) elastography are collected at day 1, week 24, week 52 and every 12 months. Clinical scores of fibrosis (e.g. FIB-4 and AST-to-platelet index) are calculated based on baseline characteristics and clinical chemistries.

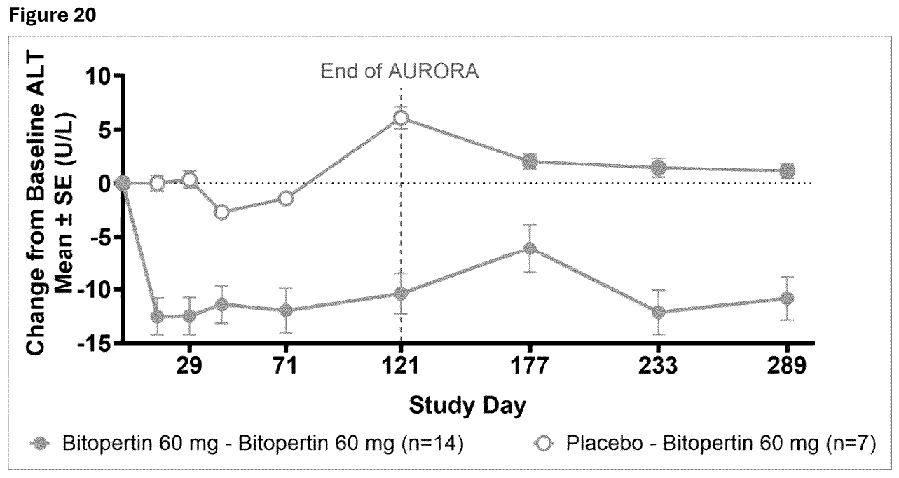

Preliminary data demonstrates that both a sensitive biomarker of hepatic health (ALT) and serum bile acids show improvement after initiation of bitopertin in patients with EPP (see FIG. 20). These data demonstrate that bitopertin can positively impact hepatic health in this population. Therefore, in addition to improvement in sun light exposure reactivity and tolerability, bitopertin may result in reduced liver damage, or even improvement in various hepatobiliary markers, in EPP patients.

### Example 8: Effect of Bitopertin on the Liver and on Levels of Protoporphyrin IX (PPIX) in Bile, Blood, Liver, and Stool in Patients with Erythropoietic Protoporphyria (EPP)/X-linked Protoporphyria (XLP) and Increased Liver Stiffness and/or Liver Enzymes at Baseline

A small percentage of patients with EPP/XLP, variously estimated at 1 to 5%, develop liver injury and fibrosis due to the excess PPIX that is constantly presented to the liver for secretion into the bile and then for excretion in the feces. Treatments aimed at increasing tolerance to sunlight such as afamelanotide are not likely to have much, if any, effect to reduce the risk of PPIX-induced liver damage, which may be rapidly fatal without liver transplantation. Provided herein is a study designed to evaluate whether bitopertin is effective in reducing hepatic and biliary levels of PPIX, which over time, with chronic and ongoing bitopertin treatment, will ameliorate and forestall progression of PPIX hepatopathy, providing an additional major benefit and reason for its chronic use in patients with EPP/XLP.

### Study Design

Provided herein is an open-label, investigator-initiated study of bitopertin (60 mg/day) in selected and carefully monitored participants with EPP or XLP who have increased liver stiffness and/or elevated liver enzymes at baseline. The target number of participants is at least 10. The overall study duration for participants is approximately 2 years, encompassing Screening (up to 28 days), the treatment period (104 weeks), and the end of study (EOS) visit (<8 days from last dose).

Study assessments will be performed at Screening and Baseline, at 6, 12, and 18 months (interim time points), and 24 months (end of the study). Assessments include elastography of the liver (by Fibroscan, magnetic resonance elastography (MRE), or Velacur/Sonic Incytes) and liver chemistries (serum albumin, ALT, AP, AST, GGT, TBR, PT/INR) (see Schedule of Assessments [SOA], Table 2). Protoporphyrin IX concentrations in total blood, in RBC, and in plasma will be measured as will be variables to allow for calculation of serum AST/platelet ratio index (APRI), enhanced liver fibrosis (ELF) test, estimate of hepatic fibrosis based upon serum ALT, AST, age, platelet count (Fib-4), and Fibrotest/Actitest.

Measurements of porphyrins in bile, obtained from the second portion of the duodenum with use of specially designed multi-lumen, oroenteric tubes and from liver biopsies, as well as hepatic histopathology, will be obtained only at baseline and at end of study, after 2 years of therapy with bitopertin.

### Inclusion Criteria

Participants must meet all the following inclusion criteria to be eligible to participate in this study.
1 Diagnosis of EEP or XLP that has been biochemically and genetically verified prior diagnosis of EPP or XLP;
2. Age 18-75 years;
3. Increased liver stiffness at baseline, defined as stiffness measurement [E value] by:
   Fibroscan >7 kPa; or
   MRE that is >3.0 kPa; or
   Velacur/Sonicincyte that is >6.0 kPa; and/or
   elevated liver enzymes: serum ALT >2×upper limit of normal (ULN), but not greater than 5×ULN and/or serum AP >2×ULN, but not greater than 5×ULN.
4. INR <1.4.
5. Compensated liver disease at baseline, defined as lack of ascites, encephalopathy, hepatocellular carcinoma, clinically evident icterus or jaundice, peripheral edema.
6. Willingness and ability to volunteer and provide informed consent for a long-term study that will include liver biopsies and collection of bile from the second portion of the duodenum, at baseline and at the end of study. In addition, follow-up visits will be planned every 26 weeks throughout the study, with plans for repeat routine/safety labs at each visit and for measures of liver stiffness and markers of hepatic status and fibrosis (Fib-4, APRI, ELF, Fibrotest) performed once every 6 months.

### Exclusion Criteria

Participants must not meet any of the following exclusion criteria to be eligible to participate in this study.
1. Chronic hepatitis B, C, D, or E;
2. Human immunodeficiency (HIV) infection;
3. Alcohol use >21 units/week for men or >14 units/week for women;
4. Any known active malignancy other than small and localized squamous or basal cell carcinoma of the skin;
5 Advanced or decompensated heart, lung, kidney, liver, or neuro-psychiatric disease;
6. Any other conditions that, in the opinion of the Investigator, renders the individual unfit to participate.

### Dose

Participants will take 60 mg of oral bitopertin, once daily (QD). A study drug diary will be dispensed to the participant at baseline. Participants will be instructed to bring their diaries with them to all study visits. Diary information will be reviewed and/or collected by the study team during study visits.

If a participant becomes intolerant of study drug, as assessed by the Investigator, study drug may be temporarily withheld. A temporary dose reduction from 60 mg to 30 mg bitopertin (2 tablets to 1 tablet) may also be permitted. The full 60 mg bitopertin QD dose may be resumed at the Investigator's discretion.

If needed, participants may also be permanently de-escalated to 30 mg bitopertin QD at their next scheduled visit (after consultation between the Investigator and Sponsor).
Study drug administration must be discontinued if any of the following events occur:
Hypersensitivity or suspected allergic reaction to the study drug
Withdrawal of consent.
At the discretion of the Investigator for situations including, but not limited to, a clinically significant event, non-compliance with treatments or procedures to the extent that study integrity is compromised, or prohibited medication exposure.
A medical emergency that requires permanent discontinuation of study drug.
A continued decrease of ≥2 g/dL hemoglobin despite study drug dose reduction.
Participant becomes pregnant
   Participants who prematurely discontinue study drug should be encouraged to continue in the study and continue protocol-required clinic visits, and safety assessments. The reason for discontinuation of study drug must be recorded in the participant's electronic case report form (eCRF).

### Study Assessments

The Primary Objective is to assess safety and tolerability of bitopertin in subjects with EPP or XLP and evidence of compensated liver disease at baseline. Primary endpoints include:
Safety and tolerability will be assessed based upon the incidence of treatment emergent adverse events (TEAE), assessed by site investigators as related to bitopertin therapy.
Compensated liver disease at baseline will be defined as lack of ascites, encephalopathy, hepatocellular carcinoma, clinically evident icterus or jaundice, peripheral edema.

To assess effects of bitopertin on levels of protoporphyrin IX (PPIX) and other porphyrins in the whole blood, plasma, and red blood cells (RBC). Secondary Endpoints include:
Percent change from baseline to 12 months (interim time point) and end of study (24 months) in levels of PPIX in whole blood, plasma, and RBC.
Percent change from baseline to 6, 12, and 18 months (interim time points) and end of study (24 months), in liver tests (serum alanine aminotransferase [ALT], aspartate aminotransferase [AST], alkaline phosphatase [AP], total bilirubin [TBR], gamma-glutamyl transpeptidase [GGT], albumin, prothrombin time [PT]/International Normalized Ratio [INR]) and selected surrogate estimates of hepatic fibrosis: serum AST/platelet ratio index (APRI), enhanced liver fibrosis (ELF) test, estimate of hepatic fibrosis based upon serum ALT, AST, age, platelet count (Fib-4), Fibrotest/Actitest.
To assess effects of bitopertin on hepatic histopathology, secondary endpoints include: change from baseline to end of study, (24 months) in histopathologic evidence of hepatic fibrosis, necro-inflammation, fat, cholestasis, bile plugs, birefringent brown deposits in the liver, or other histopathology, as estimated by an experienced hepato-pathologist (blinded as to when the biopsies were performed).
To assess effects of bitopertin on PPIX levels in bile, liver, and stool, secondary endpoints include: Percent change from baseline to 12 months (interim time point) and end of study (24 months) in liver stiffness, measured by the same instrument, each participant to serve as his or her own control; Percent change from baseline to end of study, 24 months, in biliary concentrations of PPIX and in microscopic evidence of crystals or precipitates in the bile, each participant to serve as his or her control.

To assess effects of bitopertin on bile composition and assess the effects of bitopertin treatment on Patient Reported Outcomes (PRO)/Quality of Life (QOL), exploratory endpoints include
Correlations among PPIX levels collected from the second portion of duodenum to those found in plasma, RBCs, stool collected on the days (±7 days) and in the liver. Collections from the second portion of the duodenum to be performed by specially designed oroenteric tubes at baseline and at the end of study.
Change from baseline in PRO scores from: Patient-Reported Outcomes Measurement Information System (PROMIS).

The study includes blood draws which should occur pre-dose and at the same time of day at each scheduled assessment (except for post-dose PK sampling). Scheduled assessment times for clinical chemistry and hematology will be at all study visits except enrollment and EOS Visit.

Hematology parameters include a complete white blood cell count (WBC) with differential, red RBC, hemoglobin, hematocrit, platelet count, mean corpuscular volume (MCV), red cell distribution width (RDW), mean corpuscular hemoglobin concentration (MCHC), and mean corpuscular hemoglobin (MCH). Special hematology parameters include reticulocyte (absolute) and reticulocyte %, and reticulocyte hemoglobin concentration (CHr).

Chemistries include serum sodium, potassium, carbon dioxide, chloride, blood urea nitrogen (BUN), creatinine, glucose, calcium, AP, total bilirubin, direct bilirubin, AST, ALT, GGT, albumin, and phosphorus.

Liver disease assessments will be performed at Screening, Baseline, at 6, 12, and 18 months (interim time points), and at the end of the study (24 months). These will include elastography of the liver by Fibroscan (or MRE, or Velacur/Sonic Incytes if FibroScan is unavailable) and liver chemistries (serum albumin, ALT, AP, AST, GGT, TBR, PT/INR).

Throughout the study, hepatic fibrosis will be assessed as described in this section. The timing of these assessments is provided in the SoA (Table 2).

The disclosure provides an ELF™ test performed on serum [2.5 mL preferred, minimum volume 1.0 mL]. It consists of 3 fully automated, 2-site sandwich immunoassays using direct chemiluminometric technology. The component assays use 2 monoclonal mouse antibodies: The first antibody is an acridinium ester-labeled antibody; the second is a biotin-labeled antibody. The solid phase contains streptavidin-coated paramagnetic particles. The ELF™ score is derived from an algorithm that combines results for type III procollagen peptide (PIIINP), tissue inhibitor of metalloproteinase-1 (TIMP-1), and hyaluronic acid (HA). ELF™ is a trademark of Siemens Healthcare Diagnostics.

Risk cut-offs to assess the likelihood of progression to cirrhosis and liver-related clinical events within 3.9 years following baseline ELF score (IQR: 14.0-22.4 months: Table 18):

TABLE 18

| | |
|---|---|
| Lower risk | <9.80 |
| Mid risk | 9.80-11.29 |
| Higher risk | >11.29 |

Note:
The ELF ™ Score is a unitless numerical value.

APRI is a serum AST to platelet count ratio index. It is calculated as follows: AST [IU/L]/40 divided by platelet count [10-9/L]×100. In a meta-analysis of 40 studies, investigators concluded that an APRI score greater than 1.0 had a sensitivity of 76% and specificity of 72% for predicting cirrhosis. In addition, they concluded that an APRI score greater than 0.7 had a sensitivity of 77% and specificity of 72% for predicting significant hepatic fibrosis. For detection of cirrhosis, using an APRI cutoff score of 2.0 was more specific (91%) but less sensitive (46%). The lower the APRI score (less than 0.5), the greater the negative predictive value (and ability to rule out cirrhosis) and the higher the value (greater than 1.5) the greater the positive predictive value (and ability to rule in cirrhosis); midrange values are less helpful.

Fib-4 is another method to estimate hepatic fibrosis. It uses the age of the participant in years, serum AST [U/L], serum ALT [U/L], and platelet count [x 10-9/L or 10-3/□L]. Higher numbers suggest higher likelihood of hepatic fibrosis or cirrhosis.

Porphyrins, including whole blood metal-free PPIX, plasma PPIX, RBC PPIX, and RBC zinc protoporphyrin (ZnPPIX). All samples for PPIX measurements will be wrapped in Al foil immediately after being obtained because of known sensitivity of metal-free PPIX to light. Measurements of porphyrins in bile, obtained from the second portion of the duodenum with use of specially designed multi-lumen, oroenteric tubes (Appendix 3) and from liver biopsies (Appendix 4), as well as hepatic histopathology, will be obtained only at baseline and at end of study, after 24 months of therapy with bitopertin.

The Fibrotest (Fibrosure) is a test algorithm developed and patented by BioPredictive. It combines the results of 5 biomarkers (GGT, TBR, alpha-2 macroglobulin, apolipoprotein A1, and haptoglobin) with weighting for participant age and gender. The Actitest adds serum ALT as a marker of hepatic inflammatory activity.

Participants will be asked to complete patient-reported outcome (PRO) and quality of life (QOL) measures, including Patient Global Impression of Severity (PGIS) and Patient Global Impression of Change (PGIC) throughout the study.

Two participant health questionnaires will be completed throughout the study, the 8-item Patient Health Questionnaire (PHQ-8) and Columbia Suicide Severity Scale (C SSRS).

### Example 9: Use of Bitopertin in Specific Populations

### Pediatric Use

The safety and efficacy of bitopertin in patients with EPP or XLP aged 12 to less than 18 years have been established based on data from a randomized, open-label study. The safety profile of adolescent patients was similar to adults. The safety and effectiveness of bitopertin in patients with EPP or XLP younger than 12 years of age have not been established.

No differences in sensitivity or exposures were observed between juvenile and adult rats. Daily oral administration of bitopertin in juvenile rats from postnatal day (PND) 23 through PND 72 (equivalent to approximately 2 years and older in humans) revealed similar effects as observed in adult animals. The no-observed-adverse-effect level was 3 mg/kg/day in females and 12 mg/kg/day in males (1.6 and 2.0 times the MRHD, based on AUC, respectively).

### Geriatric Use

In clinical studies of bitopertin in patients with EPP or XLP, 9 (9%) patients were aged 65 years and older, and 1 (1%) patient was aged 75 years and older. Clinical studies did not include sufficient numbers of patients aged 65 years and older to determine whether they respond differently from younger adult population. The elderly patient population may not require dose adjustment.

### Renal Impairment

One of skill in the art would appreciate that dose adjustments would not be needed in patients with mild (estimated glomerular filtration rate [eGFR] 60 to <90 mL/min/1.73 m2), moderate (eGFR 30 to <60 mL/min/1.73 m2) or severe (eGFR <30 mL/min/1.73 m2) renal impairment or end-stage renal disease.

A study comparing the pharmacokinetics of a single 20 mg dose of bitopertin in subjects with end-stage renal disease and not on dialysis (eGFR <15 mL/1.73 m2, n=8) to healthy volunteers (n=8) did not show any clinically meaningful differences in the exposure of bitopertin.

### Hepatic Impairment

Exposure to unbound bitopertin was increased in patients with severe hepatic impairment (ChildPugh Class C). The bitopertin dose administered to patients with severe hepatic impairment can be lower than in patients with normal hepatic function. Patients with severe hepatic impairment can be monitored for any adverse reactions. The dosage of bitopertin can be modified for patients with mild (Child-Pugh Class A) or moderate (Child-Pugh Class B) hepatic impairment.

In a single-dose study with bitopertin 20 mg, the unbound bitopertin exposure (AUC) was similar in subjects with mild or moderate hepatic impairment and higher (ratio of 1.7) in subjects with severe hepatic impairment when compared to healthy subjects. The Cmax of unbound bitopertin was similar in subjects with mild, moderate, or severe hepatic impairment and healthy subjects.

Physiologically based pharmacokinetic (PBPK) modeling studies with bitopertin 60 mg once-daily dosing showed a ratio of 2.0 for the AUC of unbound bitopertin in subjects with severe hepatic impairment compared to healthy controls.

The dose of bitopertin in patients with severe hepatic impairment (Child-Pugh Class C) can be 30 mg once daily. Patients with severe hepatic impairment may be monitored for any adverse reactions. The dose in patients with mild (Child-Pugh A) or moderate (Child-Pugh B) hepatic impairment can be the same as those in patients with normal hepatic function.

### Example 10: Drug Interaction Studies

### Effect of Strong and Moderate CYP3A4 Inhibitors on Bitopertin

Ketoconazole (a strong CYP3A inhibitor) increased bitopertin AUC₀₋₃₁₂ hr and Cₘₐₓ to approximately 4.2-fold and 1.2-fold, respectively, following a single dose of bitopertin 10 mg. Erythromycin (a moderate CYP3A inhibitor) increased bitopertin AUC_{0-∞} and Cₘₐₓ to approximately 2-fold and 1.2-fold, respectively, following a single dose of bitopertin 30 mg.

### Effect of Strong CYP3A4 Inducers on Bitopertin

Carbamazepine (a strong CYP3A inducer) decreased bitopertin AUC0-□ and Cₘₐₓ by approximately 80% and 35%, respectively, following a single dose of bitopertin 20 mg.

### Effect of Bitopertin on CYP2C9 and OATP Substrates

No clinically significant differences in exposure of CYP2C9 and organic anion-transporting polypeptide (OATP) substrate (such as rosuvastatin or its active metabolite, N-desmethyl rosuvastatin) were observed when used concomitantly with bitopertin.

### Effect of Bitopertin on P-Glycoprotein Substrates

Concomitant administration of bitopertin and paliperidone (a P-glycoprotein [P-gp] substrate) resulted in 22% increase in Cₘₐₓ and 11% increase in AUC_{0-∞} of paliperidone, indicating no clinically significant P-gp inhibitory potential for bitopertin.

Interaction with Alcohol

Bitopertin did not potentiate the cognitive or psychomotor effects of alcohol, and alcohol had no effect on the pharmacokinetics of bitopertin.

### In Vitro Studies

### Effect of CYP3A4 Inhibitors and Inducers:

PBPK modeling studies showed that the effect of coadministration of bitopertin 60 mg with strong or moderate CYP3A inhibitors, or strong CYP3A inducers, is consistent with the magnitudes of the effect observed in clinical studies.

### Metabolism-Related DDI Studies:

Bitopertin showed a minor potential to induce the activity of CYP450 isoenzymes CYP3A4 (≥4-fold) and CYP2B6 (≥2-fold) in human hepatocytes.

Bitopertin showed no significant inhibition of CYPs 1A2, 2B6, 2C8, 2C19, 2D6, or 3A4/5 activities at concentrations up to 50 μM. Bitopertin is a weak inhibitor of CYP2C9 (half maximal inhibitory concentration [IC50] of 11.9 μM).

### Drug Transporter-Related Studies:

Bitopertin has a low potential to inhibit transporter P-gp when using digoxin, erythromycin, or dabigatran etexilate as substrates (IC50 values ≥30 μM, 18 μM, and 7.28 UM, respectively) and higher potential to inhibit transporter P-gp using quinidine, risperidone, and paliperidone as substrates (IC50 values 0.3 to 4 μM, 1.7 μM, and 0.9 to 1.8 UM, respectively). However, in vivo studies showed no clinically relevant change in exposure (AUC0-D) for P-gp substrates (e.g., paliperidone) when co-administered with bitopertin.

Bitopertin is not a substrate for breast cancer resistance protein (BCRP) and did not meaningfully inhibit organic anion-transporter (OAT) 1, OAT3, organic cation transporter (OCT) 2, or multidrug and toxin extrusion protein (MATE) 1. However, bitopertin has modest inhibitory potential on BCRP, MATE-2K, OATP1B1, OATP1B3, and OATP2B1 although these are not considered clinically relevant. Finally, there is no meaningful active transport of bitopertin by OATP1B1 or OATP1B3.

A summary of effects/interactions with other drugs is provided in Table 19.

TABLE 19
Strong or Moderate CYP3A4 Inhibitors

| | |
|---|---|
| Clinical Impact | Co-administration of bitopertin with strong or |
| | moderate CYP3A4 inhibitors may increase |
| | the area under the concentration-time curve |
| | (AUC), which may increase the risk of |
| | bitopertin-related adverse reactions. |
| Prevention or | Co-administration of strong CYP3A4 |
| Management | inhibitors, or food or drink containing |
| | grapefruit, during treatment with bitopertin |
| | may be reduced and/or avoided. |
| | The dosage of bitopertin when co- |
| | administered with moderate CYP3A4 |
| | inhibitors, may be reduced. |

Strong CYP3A4 Inducers

| | |
|---|---|
| Clinical Impact | Co-administration of bitopertin with strong |
| | CYP3A4 inducers may decrease the AUC, |
| | and/or reduce the efficacy of bitopertin. |
| Prevention or | One of skill in the art would appreciate that |
| Management | Co-administration of strong CYP3A4 |
| | inducers with bitopertin can be limited and/or |
| | avoided. |

### Example 11: Toxicology

### Carcinogenesis

The carcinogenic potential of bitopertin was assessed in 2-year mouse and 2-year rat studies. Bitopertin was dosed orally as diet admixture in both studies. No evidence of tumorigenicity was observed in male and female mice at doses up to 80 mg/kg/day (exposure of 3.4 times the MRHD in males and 5.9 times the MRHD in females, based on AUC). No evidence of tumorigenicity was observed in rats at doses to 30 mg/kg/day in males and 8 mg/kg/day in females (exposure of 5.7 times the MRHD in males and 7.4 times the MRHD in females, based on AUC).

### Mutagenesis

Bitopertin was not mutagenic in an in vitro bacterial reverse mutation (Ames) assay and did not show chromosomal aberrations in human peripheral blood lymphocytes. Bitopertin was not clastogenic in vivo in the rat bone marrow micronucleus assay.

### Fertility

Oral administration of bitopertin to male (1, 3, and 10 mg/kg/day) and female (3, 10, and 30 mg/kg/day) rats prior to and during mating and continuing in females to Gestation Day 7 did not produce any effects on fertility or early embryonic development. The no-effect dose level (10 mg/kg/day in males and 30 mg/kg/day in females) was associated with plasma exposures (based on AUC) of approximately 4.5 and 4.4 times the MRHD, respectively.

### Example 12: Adverse Reactions

Because clinical trials are conducted under widely varying conditions, adverse reaction rates observed in the clinical trials of a drug cannot be directly compared to rates in the clinical trials of another drug and may not reflect the rates observed in practice.

The safety profile of bitopertin is based on data from two clinical studies in patients with EPP or XLP (aged 12 years and older). A total of 77 patients received bitopertin at doses up to 60 mg once daily in the AURORA trial (N=51) and the BEACON trial (N=26).

The AURORA trial is a randomized, double-blind, placebo-controlled study in which 51 adult patients with EPP (aged 18 years and older) received bitopertin at 20 mg or 60 mg once-daily doses. Among the 51 patients, 51% were female and 96% were White. Patients were aged 21 years to 75 years. The most common adverse reaction (occurring in >3 bitopertin 60 mg-treated patients and higher than placebo by ≥2%) was dizziness.

Table 20 summarizes the adverse reaction occurring in >3 bitopertin 60 mg-treated patients and at a frequency higher than placebo by ≥2% in the 17-week placebo-controlled trial (AURORA).

The BEACON trial is a randomized open-label study in which 26 patients aged 12 years and older with EPP or XLP received 20 mg or 60 mg of bitopertin once daily for 24 weeks. The safety profile for the adult and adolescent patients enrolled in the BEACON trial was similar to that observed in the AURORA trial, with dizziness as the most commonly reported (65%) adverse reaction.

TABLE 20

| Adverse Reaction | Bitopertin 60 mg | Placebo |
|---|---|---|
| (Preferred Term) | N = 25 n (%) | N = 24 n (%) |
| Dizziness | 11 (44%) | 3 (13%) |

### Example 13: In Vitro Evaluation of Bitopertin

In vitro bitopertin is a potent glycine reuptake inhibitor of the human GlyT1 with more than 1000-fold selectivity for human GlyT1 versus human GlyT2. Cellular biology assays on 17 enzyme targets and 3 neurotransmitter transporters (uptake and release) showed that bitopertin has at least 300-fold greater selectivity for GlyT1 than for these other targets. Bitopertin showed at least 700-fold greater selectivity for GlyT1 than for an additional 86 other protein targets in radioligand binding studies. In addition, bitopertin exhibited very high selectivity (by at least 2000-fold) with respect to other two glycine-related targets, the NMDA-R glycine-site and the glycine receptor as demonstrated by binding assays. Specifically, bitopertin did not show significant binding when tested up to 10 μM concentration to dopamine, norepinephrine, serotonin, GABA, acetylcholine, opioid, NMDA and cannabinoid receptors. In addition, bitopertin did not affect the uptake or the release of dopamine, serotonin and norepinephrine as assessed in brain synaptosomes. Bitopertin also did not bind to or affect the activity of other receptors, enzymes, or channels that may lead to abuse potential.

### Example 14: Evaluation of Abuse Potential of Bitopertin

The abuse potential of bitopertin was assessed in a human abuse study in recreational central nervous system-depressant drug users. This study enrolled a total of 67 participants who provided a positive differentiation of the effects of a single dose of a benzodiazepine (20 mg diazepam) compared to placebo in Part 1 of the study. Only the participants who successfully passed the prescreening qualification procedure (i.e., were able to distinguish the positive control from placebo and show greater peak liking) were enrolled in Part 2. In Part 2 of this study, these participants were administered single doses of each of the following double-dummy treatments, under fasted conditions: 10 mg, 40 mg, or 120 mg of bitopertin and 20 mg of diazepam (active control) using a double-blind, placebo-controlled, and positive-controlled randomized crossover design. The study included a washout period of at least 10 days (maximum 21 days) between each treatment period. The range of doses of bitopertin used in this study support the requirements of the FDA Guidance for Industry: Assessment of Abuse Potential of Drugs (January 2017).

Diazepam was considered an appropriate positive control because it has an adverse effect profile comparable to bitopertin (with respect to somnolence and dizziness) and has PK characteristics similar to bitopertin, with peak concentrations at 1 to 1.5 hours and a terminal half-life of approximately 2 days. Doses of 10, 40, and 120 mg bitopertin all demonstrated a significantly lower abuse potential than diazepam 20 mg (1-sided p-value <0.0001), as assessed by the visual analog scale (VAS) “drug liking at the moment” and “drug liking overall.” The comparison with placebo also indicated that bitopertin has no overall abuse potential.

Additional evaluations included VAS (bipolar) for “alertness/drowsiness”; VAS (unipolar scales) for “drug high” (measures euphoria); and Addiction Research Center Inventory (ARCI) for the subscales MBG (Morphine Benzedrine Group scale, measures euphoria), PCAG (Pentobarbital Chlorpromazine Alcohol Group scale, measures sedation), BG (Benzedrine Group scale, measures stimulation), LSD (Lysergic Acid Diethylamide Group scale, measures dysphoria and agitation), and A (Amphetamine scale).

The pattern in the A, BG, LSD, and MBG subscales of ARCI for all bitopertin doses was comparable to that of placebo. However, bitopertin did result in a dose-dependent increase in the subjective sensation of sedation as measured by the PCAG subscale. Nevertheless, for the assessments indicative of sedation and drowsiness (ARCI-PCAG and VAS “alertness/drowsiness”), the response (difference to placebo) after 120 mg bitopertin was approximately 50% to 60% of the diazepam effect. Collectively, the data confirm that bitopertin does not induce any subjective feeling of euphoria, dysphoria/agitation, stimulation, or amphetamine-like effect.

Based upon this study, all doses of bitopertin showed significantly less drug liking potential than diazepam (positive control). Compared to placebo, bitopertin exhibited similar drug liking potential to placebo at the 10 mg and 40 mg dose; only the high (120 mg) bitopertin dose had the upper limit of the 95% CI marginally above the non-inferiority margin. As a result, the overall bitopertin drug liking response in the human abuse study is comparable to that of unscheduled drugs. Summary data from the human abuse study is provided in Table 21.

TABLE 21

| Balance of Effects | | | | | | | | | |
|---|---|---|---|---|---|---|---|---|---|
| Drug | | | | | | | Positive | | |
| Liking at | Overall | Take | Sedative Effects | | Stimulant | | Effects | | Negative |
| the | Drug | Drug | Alertness/ | | Effects | | Drug | | Effects |
| Moment | Liking | Again | Drowsiness | ARCI | ARCI | ARCI | High | ARCI | ARCI |
| VAS | VAS | VAS | VAS | PCAG | BG | A | VAS | MBG | LSD |

Bitopertin vs Diazepam 20 mg

| | | | | | | | | | | |
|---|---|---|---|---|---|---|---|---|---|---|
| Diazepam | 20.01* | 19.96* | 36.34* | −20.44* | 11.45* | −5.24 | 2.36* | 45.66* | 6.65* | 3.58* |
| vs Bito 10 | | | | | | | | | | |
| mg | | | | | | | | | | |
| Diazepam | 15.75* | 15.71* | 26.15* | −14.38* | 7.81* | −4.62* | 1.72* | 33.98* | 5.58* | 2.58* |
| vs Bito 40 | | | | | | | | | | |
| mg | | | | | | | | | | |
| Diazepam | 13.03* | 13.03* | 22.56* | −11.45* | 4.89* | −3.40* | 2.51* | 24.5* | 7.2* | 2.73* |
| vs Bito | | | | | | | | | | |
| 120 mg | | | | | | | | | | |

Bitopertin vs placebo

| | | | | | | | | | | |
|---|---|---|---|---|---|---|---|---|---|---|
| Bito 10 | −10.19 | −9.15 | 5.46 | −2.18 | −0.09 | −0.06 | −0.05 | 0.8 | −0.08 | −0.05 |
| mg vs. | | | | | | | | | | |
| Placebo | | | | | | | | | | |
| Bito 40 | −5.92 | −6.20 | 15.65 | −8.23* | 3.55* | −0.68 | 0.59 | 12.48* | 0.99 | 0.95 |
| mg vs. | | | | | | | | | | |
| Placebo | | | | | | | | | | |
| Bito 120 | −3.2 | −5.18 | 19.23 | −11.17* | 6.47* | −1.90* | −0.2 | 21.96* | −0.63 | 0.8 |
| mg vs. | | | | | | | | | | |
| Placebo | | | | | | | | | | |

ANCOVA = analysis of covariance;
ANOVA = analysis of variance;
ARCI = Addiction Research Center Inventor;
LSD = Lysergic Acid Diethylamide;
MBG = Morphine Benzedrine Group;
PCAG = Pentobarbital Chlorpromazine Alcohol Group;
VAS = Visual Analogue Scale
Note:
Cells with unbolded values suggest no drug-liking potential either due to significant differences relative to positive control (diazepam) or NS results relative to placebo. Cells with bolded & italicized values suggest drug-liking potential due to not meeting noninferiority threshold or significant results vs placebo.
*p < 0.05, ANOVA or ANCOVA

While preferred embodiments of the present disclosure have been shown and described herein, it will be evident to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### INCORPORATION BY REFERENCE

All references cited in this disclosure, and their references, are incorporated by reference herein in their entirety where appropriate for teachings of additional or alternative details, features, and/or technical background.

What is claimed is:

1. A method of treating erythropoietic protoporphyria (EPP) or X-linked protoporphyria (XLPP) comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

2. A method of treating EPP or XLPP comprising administering 60 mg of bitopertin to a subject in need thereof for a treatment period of at least 17 weeks thereby resulting in a statistically significant improvement in pain-free light exposure in the subject.

3. A method of increasing sunlight tolerance in a subject with EPP or XLPP comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

4. A method of reducing liver damage in a subject with EPP or XLPP comprising administering 60 mg of bitopertin to a subject in need thereof for a treatment period of at least 17 weeks thereby reducing any one of i) the rate of phototoxic reactions compared to the rate observed in treatment with placebo by 75% or more, or ii) reducing alanine aminotransferase (ALT) levels in the subject by at least 5%.

5. A method of reducing protoporphyrin IX levels in a subject with EPP or XLPP comprising administering 60 mg of bitopertin to a subject in need thereof for a treatment period of at least 17 weeks thereby reducing any one or both of i) the rate of phototoxic reactions compared to the rate observed in treatment with placebo by 75% or more, or ii) reducing alanine aminotransferase (ALT) levels in the subject by at least 5%.

6. A method of treating erythropoietic protoporphyria (EPP) or X-linked protoporphyria (XLPP) in a subject in need thereof comprising
a) administering 30 mg of bitopertin to the subject for at least 14 days; and
b) administering 60 mg of bitopertin to the subject for a treatment period of at least 17 weeks;
thereby reducing any one or both of i) the rate of phototoxic reactions compared to the rate observed in treatment with placebo by 75% or more, or ii) reducing alanine aminotransferase (ALT) levels in the subject by at least 5%.

7. The method any one of claims 1-6, wherein average daily time of pain-free light exposure is increased by at least 20%.

8. The method of any one of claims 1-6, wherein the improvement in pain-free light exposure occurs after at least 4 weeks of treatment.

9. The method of any one of claims 1-6, wherein the improvement in pain-free light exposure is sustained for 2 weeks.

10. A method of treating EPP or XLPP comprising administering 60 mg of bitopertin to a subject in need thereof for a treatment period of at least 17 weeks wherein the subject's heme and hemoglobin levels are maintained in comparison to reduced levels of heme and hemoglobin observed in a healthy subject administered bitopertin.

11. A method of treating EPP or XLPP comprising administering 60 mg of bitopertin to a subject in need thereof for at least 17 weeks wherein the subject maintains liver function.

12. A method of treating EPP comprising administering 60 mg of bitopertin to a subject age 12 and older in need thereof for at least 17 weeks thereby resulting in one or more of the following:
a. a reduction in PPIX levels;
b a reduction in the rate of phototoxic reactions;
c. an increase in average time of pain-free light exposure;
d. an increase in average time to prodrome;
e. an increase in proportion of prodrome-free weekly light exposure challenges;
f. an increase in mean cumulative total time in light without pain;
g. an improvement in the EPP Impact Questionnaire (EPIQ);
h. an improvement in the Patient Global Impression of Pain (PGIC); and
i. an improvement in the Patient Global Impression of Severity (PGIS).

13. A method of treating EPP comprising administering 60 mg of bitopertin to a geriatric subject in need thereof for at least 17 weeks thereby resulting in one or more of the following:
a. a reduction in PPIX levels;
b. a reduction in the rate of phototoxic reactions;
c. an increase in average time of pain-free light exposure;
d. an increase in average time to prodrome;
e. an increase in proportion of prodrome-free weekly light exposure challenges;
f. an increase in mean cumulative total time in light without pain;
g an improvement in the EPP Impact Questionnaire (EPIQ);
h. an improvement in the Patient Global Impression of Pain (PGIC); and
i. an improvement in the Patient Global Impression of Severity (PGIS).

14. The method of any preceding claim, wherein the 60 mg of bitopertin is administered as two 30 mg doses.

15. The method of any preceding claim, wherein the bitopertin is administered once daily.

16. A method of treating EPP or XLPP in a subject in need thereof comprising administering to the subject a daily therapeutically effective dosage of bitopertin for a treatment period of at least 6 weeks, wherein pain free light exposure is increased to 5 or more hours compared to observed pain free light exposure in treatment with placebo.

17. A method of treating EPP or XLPP in a subject comprising administering a daily therapeutically effective dosage of bitopertin for a treatment period of at least 17 weeks, wherein whole blood PPIX is decreased and no dose dependent decrease in hemoglobin is observed compared to treatment in a subject without EPP.

18. The method of claim 6, wherein the subject is a pediatric subject.

19. The method of claim 18, wherein the subject is less than 18 years of age.

20. The method of claim 18, wherein the subject is between 12 and 18 years of age.

21. A method of treating EPP or XLPP in a pediatric subject comprising administering a therapeutically effective dosage of bitopertin.

22. The method of claim 21, wherein the subject is less than 18 years of age.

23. The method of claim 21 or 22, wherein the subject is between 12 and 18 years of age.

24. The method of any one of claims 21-23, wherein the therapeutically effective dosage of bitopertin is 30 mg of bitopertin.

25. The method of any one of claims 21-24, wherein the daily therapeutically effective dosage of 30 mg of bitopertin is administered to the subject daily for at least 14 days.

26. The method of claim 25, wherein the therapeutically effective dosage of bitopertin is increased to 60 mg of bitopertin after at least 14 days.

27. The method of any preceding claim, wherein the amount of or therapeutically effective dosage of bitopertin is modified for concomitant use with one or more CYP3A4 inhibitors.

28. The method of claim 27, wherein the amount of or therapeutically effective dosage of bitopertin is reduced to 30 mg of bitopertin for concomitant use with one or more CYP3A4 inhibitors.

29. The method of claim 27 or 28, wherein the one or more CYP3A4 inhibitors is a moderate CYP3A4 inhibitor.

30. The method of claim 27 or 28, wherein the one or more CYP3A4 inhibitors is a strong CYP3A4 inhibitor.

31. The method of any one of claims 27-30, wherein the one or more CYP3A4 inhibitors is selected from the group consisting of: ketoconazole, erythromycin, and carbamazepine.

32. The method of any preceding claim, wherein the subject has hepatic impairment, and wherein the subject is administered a modified amount of or modified therapeutically effective dosage of 30 mg of bitopertin once daily.

33. The method of claim, wherein the mild hepatic impairment is classified based upon the Child-Pugh scoring system.

34. The method of claim 33, wherein the hepatic impairment is mild hepatic impairment.

35. The method of claim 33 or 34, wherein the subject has a Child-Pugh score of 5 to 6.

36. The method of any one of claims 33-35, wherein the subject is in Child-Pugh class A.

37. The method of claim 33, wherein the hepatic impairment is moderate hepatic impairment.

38. The method of claim 33 or 37, wherein the subject has a Child-Pugh score of 7 to 9.

39. The method of claim 33, 37, or 38, wherein the subject is in Child-Pugh class B.

40. The method of claim 33, wherein the hepatic impairment is severe hepatic impairment.

41. The method of claim 33 or 40, wherein the subject has a Child-Pugh score of 10 to 15.

42. The method of claim 33, 40, or 41, wherein the subject is in Child-Pugh class C.

43. The method of any preceding claim, wherein a median maximum pain score due to phototoxic reactions over the treatment period is reduced.

44. The method of claim 43, wherein the median maximum pain score due to phototoxic reactions is reduced by at least 1 unit on a pain score rating scale.

45. The method of any preceding claim, wherein a number of phototoxic reactions over the treatment period is reduced.

46. The method of claim 45, wherein the number of phototoxic reactions over the treatment period is reduced by at least 50%.

47. The method of any preceding claim, wherein one or more complications of EPP or XLPP are treated or prevented.

48. The method of any one of claims 1-46, wherein progression rate and/or severity of one or more complications of EPP or XLPP are reduced.

49. The method of claim 47 or 48, wherein the one or more complications of EPP, XLPP, or CEP is selected from the group consisting of: edema, erythema, anemia, hypochromic anemia, hemolytic anemia, hemolysis, mild hemolysis, severe hemolysis, chronic hemolysis, hypersplenism, palmar keratoderma, bullae, lesions, scarring, deformities, loss of fingernails, loss of digits, cholestasis, cytolysis, gallstones, cholestatic liver failure, cholelithiasis, mild liver disease, deteriorating liver disease, terminal phase liver disease, erythrodontia, hypercellular bone marrow, myelodysplasia, thrombocytopenia, hydrops fetalis and/or death in utero.

50. The method of any preceding claim, wherein the subject has EPP.

51. The method of any one of claims 1-49, wherein the subject has XLPP.

52. The method of any preceding claim, wherein the bitopertin is administered orally.

53. The method of any preceding claim, wherein the bitopertin is formulated as an immediate release tablet.

54. The method of claim 53, wherein the immediate release tablet comprises one or more components selected from the group consisting of lactose monohydrate, maize starch, croscarmellose sodium, povidone K30, microcrystalline cellulose, talc, magnesium stearate, water, and a film coating comprising one or more of partially hydrolyzed polyvinyl alcohol, macrogol/polyethylene glycol, cellulose, talc, titanium dioxide, and iron oxide yellow.

55. The method of claim 53 or 54, wherein the immediate release tablet comprises 30 mg of bitopertin.

56. The method of any preceding claim, wherein the subject does not experience changes in skin pigmentation.

57. The method of any preceding claim, wherein the subject experiences less than 5% of adverse events associated with anemia.

58. The method of claim 28, wherein the adverse events associated with anemia are selected from shortness of breath, difficulty breathing, weakness, fatigue, dizziness, lightheadedness, headaches, nausea, irregular heartbeat, chest pain, tinnitus, swelling, and/or jaundice.

59. The method of any preceding claim, wherein the subject experiences less than 5% of adverse events associated with the central nervous system (CNS).

60. The method of claim 59, wherein the adverse events associated with the CNS are selected from drowsiness, dizziness, impaired concentration, vertigo, headaches, nausea, insomnia, ataxia, and coma.

61. The method of any preceding claim, wherein the administration ameliorates hepatic toxicity.

62. The method of any preceding claim, wherein the administration reduces the incidence of hepatobiliary disease.

63. The method of any preceding claim, wherein the administration reduces the progression of hepatobiliary disease.

64. The method of any preceding claim, wherein the subject's PPIX levels decrease by at least 50% (e.g., 50%, 55%, 60%, 65%, 70%, 75%, 80%, 85%, 90%, 95%, or at least 100%) and the subject's heme levels decrease no more than 10%.

65. The method of any preceding claim, wherein the subject's PPIX levels decrease by at least 50% (e.g., 50%, 55%, 60%, 65%, 70%, 75%, 80%, 85%, 90%, 95%, or at least 100%) and the subject's hemoglobin levels decrease no more than 10%.

66. The method of any preceding claim, wherein the subject's heme levels decrease no more than 10%.

67. The method of any preceding claim, wherein the subject's PPIX levels decrease while the subject's heme levels are substantially maintained.

68. The method of any preceding claim, wherein the subject's hemoglobin levels decrease no more than 10%.

69. The method of any preceding claim, wherein the subject's PPIX levels decrease while the subject's hemoglobin levels are substantially maintained.

70. The method of any preceding claim, wherein the subject has increased free-protoporphyrin IX levels in erythrocytes.

71. The method of any preceding claim, wherein the method decreases protoporphyrin IX levels in the erythrocytes of the subject.

72. The method of any preceding claim, wherein the method decreases free-protoporphyrin IX levels in the subject.

73. The method of any preceding claim, wherein the method decreases free-protoporphyrin IX levels in the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

74. The method of any preceding claim, wherein the subject has increased protoporphyrin IX levels in stool.

75. The method of any preceding claim, wherein the method decreases protoporphyrin IX levels in stool of the subject.

76. The method of any preceding claim, wherein the method decreases protoporphyrin IX levels in the stool of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

77. The method of any preceding claim, wherein protoporphyrin IX (PPIX) synthesis is inhibited in vivo.

78. The method of any preceding claim, wherein the subject's plasma porphyrin fluoresces at a peak of 634 nm when illuminated with blue light (e.g., 400-420 nm light).

79. The method of any preceding claim, wherein the subject's plasma porphyrin fluoresces at a peak between 626 nm and 634 nm when illuminated with blue light (e.g., 400-420 nm light).

80. The method of any preceding claim, wherein the subject's skin porphyrin fluoresces at a peak of 632 nm when illuminated with blue light (e.g., 400-420 nm light).

81. The method of any preceding claim, wherein the subject's skin porphyrin fluoresces at a peak between 626 nm and 634 nm when illuminated with blue light (e.g., 400-420 nm light).

82. The method of any preceding claim, wherein the subject has increased protoporphyrin IX levels in the skin.

83. The method of any preceding claim, wherein the method decreases protoporphyrin IX levels in the skin of the subject.

84. The method of any preceding claim, wherein the method decreases protoporphyrin IX levels in the skin of the subject by at least 10% (e.g., 10%, 15%, 20%, 25%, 30%, 35%, 40%, 45%, 50%, 55%, 60%, 65%, 70%, 75%, 80%, 85%, 90%, 95%, or at least 100%).

85. The method of any preceding claim, wherein the subject has greater than 0.2 FluoDerm Units (FDU) of protoporphyrin IX levels in the skin.

86. The method of any preceding claim, wherein the subject has greater than 1.0 FDU of protoporphyrin IX levels in the skin.

87. The method of any preceding claim, wherein the subject has between 1.0 FDU and 2.5 FDU of protoporphyrin IX levels in the skin.

88. The method of any preceding claim, wherein the subject has greater than 2.5 FDU of protoporphyrin IX levels in the skin.

89. The method of any preceding claim, wherein the method decreases protoporphyrin IX levels in the skin of the subject to less than 0.5 FDU.

90. The method of any preceding claim, wherein the method decreases protoporphyrin IX levels in the skin of the subject to less than 1.0 FDU.

91. The method of any preceding claim, wherein the method decreases protoporphyrin IX levels in the skin of the subject to less than 1.5 FDU.

92. The method of any preceding claim, wherein the method decreases protoporphyrin IX levels in the skin of the subject to less than 2.0 FDU.

93. The method of any preceding claim, wherein the method decreases protoporphyrin IX levels in the skin of the subject to less than 2.5 FDU.

94. The method of any preceding claim, wherein zinc protoporphyrin IX (ZPPIX) synthesis is inhibited in vivo.

95. The method of any preceding claim, wherein 5-aminolevulinic acid (5-ALA) synthesis is inhibited in vivo.

96. The method of any preceding claim, wherein the subject in need thereof is 12 years and older.

97. The method of any one of claims 1-95, wherein the subject in need thereof is between 12 and 18 years old.

98. The method of any one of claims 1-95, wherein the subject in need thereof is a geriatric subject.

99. The method of any preceding claim, wherein the total pain-free time in light of the subject increases as compared to the total pain-free time in light prior to treatment.

100. A method of treating liver disease associated with EPP or XLPP in a subject comprising administering 60 mg of bitopertin to a subject to a subject in need thereof for a treatment period of at least four weeks.

101. A method of treating protoporphyrin IX (PPIX) induced liver damage comprising administering 60 mg of bitopertin to a subject in need thereof for a treatment period of at least four weeks.

102. A method of treating hepatopathy comprising administering 60 mg of bitopertin to a subject in need thereof for a treatment period of at least four weeks.

103. A method of decreasing liver protoporphyrin IX concentrations comprising administering 60 mg of bitopertin to a subject in need thereof for a treatment period of at least four weeks.

104. A method of reducing hepatobiliary biomarkers of liver damage comprising administering 60 mg of bitopertin to a subject in need thereof for a treatment period of at least four weeks.

105. The method of any one of claims 100-104, wherein the subject has erythropoietic protoporphyria (EPP).

106. The method of any one of claims 100-104, wherein the subject has X-linked protoporphyria (XLPP).

107. The method of any one of claims 100-106, wherein the bitopertin is administered orally.

108. The method any one of claims 100-107, wherein the bitopertin is formulated as an immediate release tablet.

109. The method of claim 79 wherein the immediate release tablet comprises one or more components selected from the group consisting of lactose monohydrate, maize starch, croscarmellose sodium, povidone K30, microcrystalline cellulose, talc, magnesium stearate, water, and a film coating comprising one or more of partially hydrolyzed polyvinyl alcohol, macrogol/polyethylene glycol, cellulose, talc, titanium dioxide, and iron oxide yellow.

110. The method of claim 108 or 109 wherein the immediate release tablet comprises 30 mg of bitopertin.

111. The method of any one of claims 100-110, wherein administration alleviates one or more symptoms of liver disease.

112. The method of claim 111, wherein the one or more symptoms of liver disease is selected from the group consisting of ascites, encephalopathy, hepatocellular carcinoma, jaundice, and peripheral edema.

113. The method of any one of claims 100-112, wherein the subject has or is at risk of developing hepatic fibrosis.

114. The method of any one of claims 100-113, wherein the subject is at risk of progression to cirrhosis.

115. The method of claim 114, wherein the subject has a low-risk, mid-risk, or high-risk of progression to cirrhosis.

116. The method of claim 114 or 115, wherein administration decreases the risk of progression to cirrhosis.

117. The method of any one of claims 114-116, wherein the risk of progression to cirrhosis is determined with an Enhanced Liver Fibrosis (ELF) test.

118. The method of claim 117, wherein the subject has an ELF score of less than 9.80.

119. The method of claim 117, wherein the subject has an ELF score between 9.80 and 11.29.

120. The method of claim 117, wherein the subject has an ELF score greater than 11.29.

121. The method of any one of claims 117-120, wherein the method decreases the subject's ELF score to less than 9.80.

122. The method of any one of claims 117-121, wherein the method decreases the subject's ELF score to less than 11.29.

123. The method of any one of claims 114-122, wherein the method prevents or delays advancement from low-risk to mid-risk of progression to cirrhosis.

124. The method of any one of claims 114-123, wherein the method prevents or delays advancement from mid-risk to high-risk of progression to cirrhosis.

125. The method of any one of claims 113-124, wherein the severity of hepatic fibrosis is determined via ultrasound elastography.

126. The method of any one of claims 113-125, wherein the severity of hepatic fibrosis is determined via magnetic resonance elastography.

127. The method of any one of claims 113-126, wherein the severity of hepatic fibrosis is determined via FibroScan.

128. The method of claim 125 or 126, wherein the subject has a hepatic fibrosis score of F0, F1, F2, F3, or F4.

129. The method of claim 128, wherein the subject has a hepatic fibrosis score of F0 and has no scarring of the liver.

130. The method of claim 128, wherein the subject has a hepatic fibrosis score of F1 and has mild scarring of the liver.

131. The method of claim 128, wherein the subject has a hepatic fibrosis score of F2 and has moderate scarring of the liver.

132. The method of claim 128, wherein the subject has a hepatic fibrosis score of F3 and has severe scarring of the liver.

133. The method of claim 128, wherein the subject has a hepatic fibrosis score of F4 and has cirrhosis of the liver.

134. The method of any one of claims 128-133, wherein the method decreases the subject's hepatic fibrosis score from F4 to F3.

135. The method of any one of claims 128-134, wherein the method decreases the subject's hepatic fibrosis score from F3 to F2.

136. The method of any one of claims 128-135, wherein the method decreases the subject's hepatic fibrosis score from F2 to F1.

137. The method of any one of claims 128-136, wherein the method decreases the subject's hepatic fibrosis score from F1 to F0.

138. The method of any one of claims 128-137, wherein the method prevents or delays progression of the subject's hepatic fibrosis score from F1 to F2, F3, or F4.

139. The method of any one of claims 128-138, wherein the method prevents or delays progression of the subject's hepatic fibrosis score from F2 to F3 or F4.

140. The method of any one of claims 128-139, wherein the method prevents or delays progression of the subject's hepatic fibrosis score from F3 to F4.

141. The method of any one of claims 125-140, wherein the subject has a liver stiffness greater than 2 kPa.

142. The method of any one of claims 125-141, wherein the subject has a liver stiffness greater than 7 kPa.

143. The method of any one of claims 125-142, wherein the subject has a liver stiffness greater than 9 kPa.

144. The method of any one of claims 125-143, wherein the subject has a liver stiffness greater than 11 kPa.

145. The method of any one of claims 125-144, wherein the subject has a liver stiffness greater than 14 kPa.

146. The method of any one of claims 125-145, wherein the subject has a liver stiffness greater than 17 kPa.

147. The method of any one of claims 141-146, wherein the method decreases the subject's liver stiffness to less than 17 kPa.

148. The method of any one of claims 141-147, wherein the method decreases the subject's liver stiffness to less than 14 kPa.

149. The method of any one of claims 141-148, wherein the method decreases the subject's liver stiffness to less than 11 kPa.

150. The method of any one of claims 141-149, wherein the method decreases the subject's liver stiffness to less than 9 kPa.

151. The method of any one of claims 141-150, wherein the method decreases the subject's liver stiffness to less than 7 kPa.

152. The method of any one of claims 141-151, wherein the method delays or prevents increases in the subject's liver stiffness.

153. The method of any one of claims 100-152, wherein the subject, when compared to baseline, has elevated levels one or more hepatobiliary biomarkers selected from the group consisting of aspartate aminotransferase (AST), alanine aminotransferase (ALT), alkaline phosphatase (AP), Gamma-glutamyl transpeptidase (GGT), bilirubin, alpha-2 macroglobulin (A2M), apolipoprotein A1 (ApoA1), haptoglobin (Hp), albumin, 5′-nucleotidase (5′-NT), and primary serum bile acids.

154. The method of claim 153, wherein the method reduces serum concentrations of primary bile acids.

155. The method of claim 154, wherein the method reduces serum primary bile acid levels by at least 5% in the subject.

156. The method of claim 154 or 155, wherein the method reduces serum primary bile acid levels by at least 10% in the subject.

157. The method of any one of claims 153-156, wherein the method reduces serum primary bile acid levels by at least 15% in the subject.

158. The method of any one of claims 153-157, wherein the method reduces ALT levels in the subject by at least 5%.

159. The method of any one of claims 153-158, wherein the method reduces ALT levels in the subject by at least 10%.

160. The method of any one of claims 153-159, wherein the method reduces ALT levels in the subject by at least 15%.

161. The method of any one of claims 153-160, wherein the method reduces AST levels in the subject by at least 5%.

162. The method of any one of claims 153-161, wherein the method reduces AST levels in the subject by at least 10%.

163. The method of any one of claims 153-162, wherein the method reduces AST levels in the subject by at least 15%.

164. The method of any one of claims 153-163, wherein the method reduces AP concentrations in the subject by at least 5%.

165. The method of any one of claims 153-164, wherein the method reduces AP concentrations in the subject by at least 10%.

166. The method of any one of claims 153-165, wherein the method reduces AP concentrations in the subject by at least 15%.

167. The method of any one of claims 153-166, wherein the method reduces GGT concentrations in the subject by at least 5%.

168. The method of any one of claims 153-167, wherein the method reduces GGT concentrations in the subject by at least 10%.

169. The method of any one of claims153-168, wherein the method reduces GGT concentrations in the subject by at least 15%.

170. The method of any one of claims153-169, wherein the method reduces serum total bilirubin (TBR) concentrations in the subject by at least 5%.

171. The method of any one of claims153-170, wherein the method reduces serum TBR concentrations in the subject by at least 10%.

172. The method of any one of claims153-171, wherein the method reduces serum TBR concentrations in the subject by at least 15%.

173. The method of any one of claims 153-172, wherein the method reduces serum A2M concentrations in the subject by at least 5%.

174. The method of any one of claims 153-173, wherein the method reduces serum A2M concentrations in the subject by at least 10%.

175. The method of any one of claims 153-174, wherein the method reduces serum A2M concentrations in the subject by at least 15%.

176. The method of any one of claims 153-175, wherein the method decreases serum ApoA1 concentrations in the subject by at least 5%.

177. The method of any one of claims 153-176, wherein the method decreases serum ApoA1 concentrations in the subject by at least 10%.

178. The method of any one of claims 153-177, wherein the method decreases serum ApoA1 concentrations in the subject by at least 15%.

179. The method of any one of claims 153-178, wherein the method increases serum Hp concentrations in the subject by at least 5%.

180. The method of any one of claims 153-179, wherein the method increases serum Hp concentrations in the subject by at least 10%.

181. The method of any one of claims 153-180, wherein the method increases serum Hp concentrations in the subject by at least 15%.

182. The method of any one of claims 153-181, wherein the method increases serum albumin levels in the subject by at least 5%.

183. The method of any one of claims 153-182, wherein the method increases serum albumin levels in the subject by at least 10%.

184. The method of any one of claims 153-183, wherein the method increases serum albumin levels in the subject by at least 15%.

185. The method of any one of claims 153-184, wherein the method reduces serum 5′-NT concentrations in the subject by at least 5%.

186. The method of any one of claims 153-185, wherein the method reduces serum 5′-NT concentrations in the subject by at least 10%.

187. The method of any one of claims 153-186, wherein the method reduces serum 5′-NT concentrations in the subject by at least 15%.

188. The method of any one of claims 114-187, wherein the subject has a FIB-4 index less than 1.00.

189. The method of any one of claims 114-188, wherein the subject has a FIB-4 index less than 1.30.

190. The method of any one of claims 114-187, wherein the subject has a FIB-4 index greater than 2.65.

191. The method of any one of claims 114-187, wherein the subject has a FIB-4 index greater than 3.25.

192. The method of any one of claims 189-191, wherein the method decreases the subject's FIB-4 index to less than 1.3.

193. The method of any one of claims 189-192, wherein the method decreases the subject's FIB-4 index to less than 2.65.

194. The method of any one of claims 189-193, wherein the method decreases the subject's FIB-4 index to less than 3.25.

195. The method of any one of claims 100-194, wherein the subject has an APRI score greater than 0.7.

196. The method of any one of claims 100-195, wherein the subject has an APRI score greater than 1.0.

197. The method of any one of claims 100-196, wherein the subject has an APRI score greater than 1.5.

198. The method of any one of claims 100-197, wherein the subject has an APRI score greater than 2.0.

199. The method of any one of claims 100-198, wherein the method decreases the subject's APRI score to less than 2.0.

200. The method of any one of claims 100-199, wherein the method decreases the subject's APRI score to less than 1.5.

201. The method of any one of claims 100-200, wherein the method decreases the subject's APRI score to less than 1.0.

202. The method of any one of claims 100-201, wherein the subject has increased concentrations of porphyrins in the liver relative to baseline.

203. The method of any one of claims 100-202, wherein the subject has increased concentration of porphyrins in bile relative to baseline.

204. The method of claim 202 or 203, wherein the porphyrins comprise PPIX.

205. The method of any one of claims 201-204, wherein the method reduces the concentration of porphyrins by at least 5%, at least 10%, at least 15%, at least 20%, at least 30%, at least 40%, or at least 50%.

206. The method of any one of claims 100-205, wherein the treatment period is at least 29 days, at least 71 days, at least 121 days, at least 177 days, at least 233 days, or at least 289 days.

207. The method of any one of claims 1-206, wherein the administration of bitopertin does not induce a statistically significant abuse potential in the subject.

208. A method of increasing sunlight tolerance in a subject with EPP or XLPP comprising administering to the subject 60 mg of bitopertin for a treatment period of at least 17 weeks resulting in a statistically significant increase in sunlight tolerance without statistically significant abuse potential.

209. A method of treating EPP or XLPP comprising administering 60 mg of bitopertin to a subject in need thereof for a treatment period of at least 17 weeks thereby resulting in a statistically significant improvement in pain-free light exposure in the subject without statistically significant abuse potential.

210. A method of reducing liver damage in a subject with EPP or XLPP comprising administering 60 mg of bitopertin to a subject in need thereof for a treatment period of at least 17 weeks, thereby reducing the rate of phototoxic reactions compared to the rate observed in treatment with placebo by 75% or more without statistically significant abuse potential.

211. A method of reducing protoporphyrin IX levels in a subject with EPP or XLPP comprising administering 60 mg of bitopertin to a subject in need thereof for a treatment period of at least 17 weeks, thereby reducing the rate of phototoxic reactions compared to the rate observed in treatment with placebo by 75% or more without statistically significant abuse potential.

212. The method of any one of claims 1-212, wherein administration of bitopertin does not induce a statistically significant abuse potential in the subject.

213. The method of any one of claims 1-212, wherein the administration of bitopertin does not induce any one or more of a drug liking response, drug liking at the moment response, or a take drug again response in the subject.

214. The method of any one of claims 1-213, wherein the administration of bitopertin does not induce overall drug liking in the subject as compared to the administration diazepam as a positive control.

215. The method of any one of claims 1-209, wherein administration of bitopertin induces an overall drug liking response comparable to placebo.

216. The method of any one of claims 1-210, wherein administration of bitopertin does not induce overall drug liking in the subject as measured by a VAS for Overall Drug Liking.

217. The method of any one of claims 1-211, wherein administration of bitopertin induces an overall drug liking response that is at least 40%, at least 50%, at least 60%, at least 70%, at least 80%, at least 90%, or at least 100% less than a positive control as measured by a VAS for Overall Drug Liking.

218. The method of claim 212, wherein the positive control comprises diazepam.

219. The method of any one of claims claim 1-218, wherein the subject experiences less than 10% of adverse events associated with the central nervous system (CNS).

220. The method of any one of claims 1-219, wherein the subject experiences less than 5% of adverse events associated with the central nervous system (CNS).

221. The method of claim 219 or 220, wherein the adverse events associated with the CNS are selected from the group consisting of drowsiness, dizziness, impaired concentration, vertigo, headaches, nausea, insomnia, ataxia, and coma.
